(19) United States
(12) Patent Application Publication
(10) Pub. No.: US 2002/0165223 A1
(43) Pub. Date: Nov. 7, 2002

(54) Substituted urea neuropeptide Y Y5 receptor antagonists

(76) Inventors: William J. Greenlee, Teaneck, NJ (US); Ying Huang, East Brunswick, NJ (US); Joseph M. Kelly, Parlin, NJ (US); Stuart W. McCombie, Caldwell, NJ (US); Andrew W. Stamford, Chatham Township, NJ (US); Yusheng Wu, New York, NY (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 09/950,908

(22) Filed: Sep. 12, 2001

Related U.S. Application Data
(60) Provisional application No. 60/232,255, filed on Sep. 14, 2000.

(51) Int. Cl. A61K 31/55; A61K 31/4535; A61K 31/4025; A61K 31/17; C 07D 4 9/02

(52) U.S. Cl. 514/217.03; 514/317; 514/326; 514/422; 514/426; 514/447; 514/596; 540/596; 546/212; 546/231; 548/527; 548/567; 564/48; 549/69

(57) ABSTRACT

Compounds represented by structural formula I

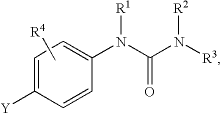

including its N-oxides wherein

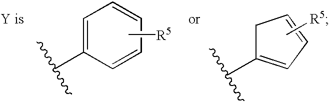

R¹ is H or (C₁-C₆)alkyl;

R² is H, (C₁-C₆)alkyl, (C₃-C₉)cycloalkyl or (C₃-C₇)cycloalkyl(C₁-C₆)alkyl;

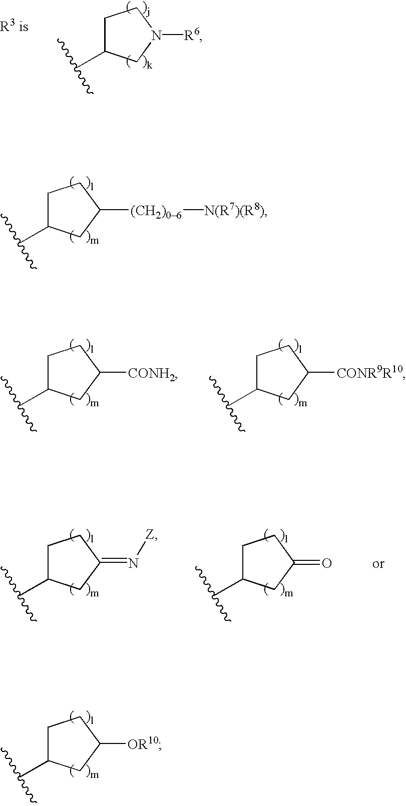

Z is OR¹⁰, —N(R⁹)(R¹⁰) or —NH₂;

j is 0, 1 or 2;

k is 1 or 2;

l is 0, 1 or 2;

m is 0, 1 or 2;

R⁴ is 1-3 substituents independently selected from the group consisting of H, —OH, halogen, haloalkyl, (C₁-C₆)alkyl, (C₃-C₇)cycloalkyl, (C₃-C₇)cycloalkyl(C₁-C₆)alkyl, —CN, —O(C₁-C₆)alkyl, —O(C₃-C₇)cycloalkyl, —O(C₁-C₆)alkyl(C₃-C₇)cycloalkyl, —S(C₁-C₆)alkyl, —S(C₃-C₇)cycloalkyl, —S(C₁-C₆)alkyl(C₃-C₇)cycloalkyl, —NH₂, —NR⁹R¹⁰, —NO₂, —CONH₂, —CONR⁹R¹⁰ and NR²COR¹⁰;

R⁵ is 1-3 substituents independently selected from the group consisting of H, halogen, —OH, haloalkyl, haloalkoxy, —CN, —NO₂, (C₁-C₆)alkyl, (C₃-C₇)cycloalkyl, (C₃-C₇)cycloalkyl(C₁-C₆)alkyl, —O(C₁-C₆)alkyl, —O(C₃-C₇)cycloalkyl, —O(C₁-C₆)alkyl(C₃-C₇)cycloalkyl, —CONH₂ and —CONR⁹R¹⁰;

R⁶ is —SO₂(C₁-C₆)alkyl, —SO₂(C₃-C₇)cycloalkyl, —SO₂(C₁-C₆)alkyl(C_{C}₇)cycloalkyl, —SO₂(C₁-C₆)haloalkyl, —SO₂(hydroxy(C₂-C₆)alkyl), —SO₂(amino(C₂-C₆)alkyl), —SO₂(alkoxy(C₂-C₆)alkyl), —SO₂(alkylamino(C₂-C₆)alkyl), —SO₂(dialkylamino(C₂-C₆)alkyl), —SO₂(aryl), —SO₂(heteroaryl), —SO₂(aryl(C₂-C₆-alkyl), SO₂N H₂, —SO₂N R⁹R¹⁰, —C(O)C₁-C₆alkyl, —C(O)C₃-C₇cycloalkyl, —C(O)aryl, —C(O)heteroaryl, —C(O)NR⁹R¹⁰, —C(O)NH₂, —C(S)NR⁹R¹⁰, —C(S)NH₂, aryl, heteroaryl, —(CH₂)ₙC(O)NH₂, —(CH₂)ₙC(O)NR⁹R¹⁰, —C(═NCN)alkylthio, —C(═NCN)NR⁹R¹⁰, (C₁-C₆)alkyl, (C₃-C₇)cycloalkyl, (C₃-C₇)cycloalkyl(C₁-C₆)alkyl, aryl(C₁-C₆)alkyl, heteroaryl(C₁-C₆)alkyl or —C(O)OR⁹, N=1 to 6;

R⁷=H or alkyl;

R⁸ is H, (C₁-C₆)alkyl, (C₃-C₇)cycloalkyl, (C₃-C₇)cycloalkyl(C₁-C₆)alkyl, aryl, heteroaryl, —SO₂(C₁-C₆)alkyl, —SO₂(C₃-C₇)cycloalkyl, —SO₂(C₁-C₆) alkyl(C₃-C₇)cycloalkyl, —SO₂(C₁-C₆)haloalkyl or —SO₂(aryl);

R⁹ is (C₁-C₆)alkyl, (C₃-C₇)cycloalkyl, (C₃-C₇)cycloalkyl(C₁-C₆)alkyl, aryl(C₁-C₆)alkyl, aryl or heteroaryl; and,

R¹⁰ is hydrogen, (C₁-C₆)alkyl, (C₃-C₇)cycloalkyl, (C₃-C₇)cycloalkyl(C₁-C₆)alkyl, aryl(C₁-C₆)alkyl, aryl or heteroaryl; or a pharmaceutically acceptable addition salt and/or hydrate thereof, or prodrug thereof,

or R⁹ and R¹⁰ taken together can form a 4-7 membered ring containing 1 or 2 heteroatoms; or where applicable, a geometric or optical isomer or a racemic mixture thereof, are claimed, as well as additional novel compounds; also claimed are pharmaceutical compositions and methods of using the aforesaid compounds in the treatment of obesity, eating disorders such as hyperphagia and diabetes.

### FIELD OF THE INVENTION

The present invention relates to neuropeptide Y Y5 receptor antagonists useful in the treatment of obesity and eating disorders, pharmaceutical compositions containing the compounds, and methods of treatment using the compounds.

### BACKGROUND OF THE INVENTION

Neuropeptide Y (NPY) is a 36 amino acid neuropeptide that is widely distributed in the central and peripheral nervous systems. NPY is a member of the pancreatic polypeptide family that also includes peptide YY and pancreatic polypeptide (Wahlestedt, C., and Reis, D., Ann. Rev. Toxicol., 32, 309, 1993). NPY elicits its physiological effects by activation of at least six receptor subtypes designated Y1, Y2, Y3, Y4, Y5 and Y6 (Gehlert, D., Proc. Soc. Exp. Biol. Med., 218, 7, 1998; Michel, M. et al., Pharmacol. Rev., 50, 143, 1998). Central administration of NPY to animals causes dramatically increased food intake and decreased energy expenditure (Stanley, B. and Leibowitz, S., Proc. Natl. Acad. Sci. USA 82: 3940, 1985, Billington et al., Am J. Physiol., 260, R321, 1991). These effects are believed to be mediated at least in part by activation of the NPY Y5 receptor subtype. The isolation and characterization of the NPY Y5 receptor subtype has been reported (Gerald, C. et al., Nature, 1996, 382, 168; Gerald, C. et al. WO 96/16542). Additionally, it has been reported that activation of the NPY Y5 receptor by administration of the Y5 —selective agonist [D-Trp³²]NPY to rats stimulates feeding and decreases energy expenditure (Gerald, C. et al., Nature, 1996, 382, 168; Hwa, J. et al., Am. J. Physiol., 277 (46), R1428, 1999). Hence, compounds that block binding of NPY to the NPY Y5 receptor subtype should have utility in the treatment of obesity, disorders such as, bulimia nervosa, anorexia nervosa, and in the treatment of disorders associated with obesity such as type II diabetes, insulin resistance, hyperlipidemia, and hypertension.

Published PCT patent application WO 00/27845 describes a class of compounds, characterized therein as spiro-indolines, said to be selective neuropeptide Y Y5 receptor antagonists and useful for the treatment of obesity and the complications associated therewith. Known urea derivatives indicated as possessing therapeutic activity are described in U.S. Pat. Nos. 4,623,662 (antiatherosclerotic agents) and 4,405,644 (treatment of lipometabolism). Provisional application, U.S. Ser. No. 60/232,255 describes a class of substituted urea neuropeptide Y Y5 receptor antagonists.

### SUMMARY OF THE INVENTION

The present invention relates to compounds represented by the structural formula I:

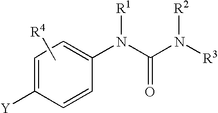

including its N-oxides, wherein Y is

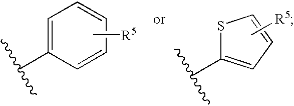

R¹ is H or (C₁-C₆)alkyl;

R² is H, (C₁-C₆)alkyl, (C₃-C₉)cycloalkyl or (C₃-C₇)cycloalkyl(C₁-C₆)alkyl;

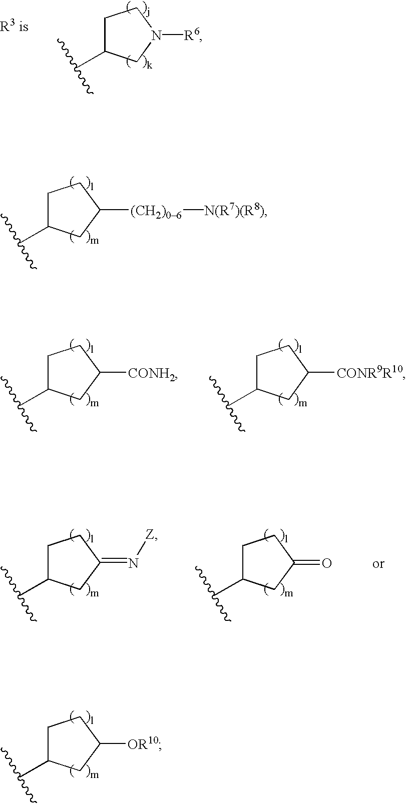

Z is OR¹⁰, —N(R⁹)(R¹⁰) or —NH₂;

j is 0, 1 or 2;

k is 1 or 2;

l is 0, 1 or 2;

m is 0, 1 or 2;

R⁴ is 1-3 substituents independently selected from the group consisting of H, —OH, halogen, haloalkyl, (C₁-C₆)alkyl, (C₃-C₇)cycloalkyl, (C₃-C₇)cycloalkyl(C₁-C₆)alkyl —CN, —O(C₁-C₆)alkyl, —O(C₃-C₇)cycloalkyl, —O(C₁-C₆)alkyl(C₃-C₇)cycloalkyl, —S(C₁-C₆)alkyl, —S(C₃-C₇)cycloalkyl, —S(C₁-C₆)alkyl(C₃-C₇)cycloalkyl, —NH₂, —NR⁹R¹⁰, —NO₂, —CONH₂, —CONR⁹R¹⁰ and NR²COR¹⁰;

R⁵ is 1-3 substituents independently selected from the group consisting of H, halogen, —OH, haloalkyl, haloalkoxy, —CN, —NO₂, (C₁-C₆)alkyl, (C₃-C₇)cycloalkyl, (C₃-C₇)cycloalkyl(C₁-C₆)alkyl, —O(C₁-C₆)alkyl, -O(C₃-C₇)cycloalkyl, —O(C₁-C₆)alkyl(C₃-C₇)cycloalkyl, —CONH₂ and —CONR⁹R¹⁰;

R⁶ is —SO₂(C₁-C₆)alkyl, —SO₂(C₃-C₇)cycloalkyl, —SO₂(C₁-C₆)alkyl(C₃-C₇)cycloalkyl, —SO₂(C₁-C₆)haloalkyl, —SO₂(hydroxy(C₂-C₆)alkyl), —SO₂(amino(C₂-C₆)alkyl), —SO₂(alkoxy(C₂-C₆)alkyl), —SO₂(alkylamino(C₂-C₆)alkyl), —SO₂(dialkylamino(C₂-C₆)alkyl, —SO₂(aryl), —SO₂(heteroaryl), —SO₂(aryl(C₂-C₆-alkyl), —SO₂NH₂, —SO₂NR⁹R¹⁰, —C(O)(C₁C₆)alkyl, —C(O)(C₃-C₇)cycloalkyl, —C(O)(C₃-C₇)cycloalkyl(C₁-C₆)alkyl, —C(O)aryl, —C(O)heteroaryl, —C(O)NR⁹R¹⁰, —C(O)NH₂, —C(S)NR⁹R¹⁰, —C(S)NH₂, aryl, heteroaryl, —(CH₂)ₙC(O)NH₂, —(CH₂)ₙC(O)NR⁹R¹⁰, —C(═NCN)alkylthio, —C(═NCN)NR⁹R¹⁰, (C₁-C₆)alkyl, (C₃-C₇)cycloalkyl, (C₃-C₇)cycloalkyl(C₁-C₆)alkyl, aryl(C₁-C₆)alkyl, heteroaryl(C₁-C₆)alkyl or —C(O)OR⁹, n=1 to 6;

R⁷=H or alkyl;

R⁸ is H, (C₁-C₆)alkyl, (C₃-C₇)cycloalkyl, (C₃-C₇)cycloalkyl(C₁-C₆)alkyl, aryl, heteroaryl, —SO₂(C₁-C₆)alkyl, —SO₂(C₃-C₇)cycloalkyl, —SO₂(C₁-C₆)alkyl(C₃-C₇)cycloalkyl, —SO₂(C₁-C₆)haloalkyl or —SO₂(aryl);

R⁹ is (C₁-C₆)alkyl, (C₃-C₇)cycloalkyl, (C₃-C₇)cycloalkyl(C₁-C₆)alkyl, aryl(C₁-C₆)alkyl, aryl or heteroaryl; and,

R¹⁰ is hydrogen, (C₁-C₆)alkyl, (C₃-C₇)cycloalkyl, (C₃-C₇)cycloalkyl(C₁-C₆)alkyl, aryl(C₁-C₆)alkyl, aryl or heteroaryl;

or R⁹ and R¹⁰ taken together can form a 4-7 membered ring containing 1 or 2 heteroatoms;

or a pharmaceutically acceptable addition salt and/or hydrate thereof, or prodrug thereof, or where applicable, a geometric or optical isomer or a racemic mixture thereof.

The present invention also relates to a method of treating obesity and eating disorders, such as hyperphagia, and diabetes comprising administering to a mammal in need of such treatment an effective amount of a compound of formula I.

Another aspect of the invention is a pharmaceutical composition for treating obesity, eating disorders and diabetes which comprises a compound of formula I in combination with a pharmaceutically acceptable carrier.

### DETAILED DESCRIPTION

Except where stated otherwise, the following definitions apply throughout the present specification and claims. These definitions apply regardless of whether a term is used by itself or in combination with other terms. Hence the definition of “alkyl” applies to “alkyl” as well as to the “alkyl” portions of “alkoxy”, etc.

Alkyl represents a straight or branched saturated hydrocarbon chain having the designated number of carbon atoms. Where the number of carbon atoms is not specified, 1 to 6 carbons are intended.

Halo represents fluoro, chloro, bromo or iodo.

Haloalkyl refers to alkyl substituted by halo, wherein the number of halo substituents ranges from one to as many halo substituents required for full substitution of the alkyl substituent.

Aryl refers to a mono- or bicyclic ring system having at least one aromatic ring including, but not limited to, phenyl, naphthyl, tetrahydronaphthyl, indanyl, and the like. The aryl group can be unsubstituted or substituted with one, two, or three substituents independently selected from lower alkyl, halo, cyano, nitro, haloalkyl, hydroxy, alkoxy, carboxy, carboxamide, mercapto, sulfhydryl, amino, alkylamino and dialkylamino.

Heteroaryl refers to 5- to 10-membered single or benzofused aromatic rings consisting of 1 to 3 heteroatoms independently selected from the group consisting of —O—, —S—, and —N═, provided that the rings do not possess adjacent oxygen and sulfur atoms. The heteroaryl group can be unsubstituted or substituted with one, two, or three substituents independently selected from lower alkyl, halo, cyano, nitro, haloalkyl, hydroxy, alkoxy, carboxy, carboxamide, mercapto, sulfhydryl, amino, alkylamino, dialkylamino.

When a variable appears more than once in the structural formula, for example R⁹, the identity to each variable appearing more than once may be independently selected from the definition for that variable.

N-oxides can form on a tertiary nitrogen present in an R substituent, or on ═N— in a heteroaryl ring substituent and are included in the compounds of formula I.

For compounds of the invention having at least one asymmetrical carbon atom, all isomers, including diastereomers, enantiomers and rotational isomers are contemplated as being part of this invention. The invention includes d and I isomers in both pure form and in admixture, including racemic mixtures. Isomers can be prepared using conventional techniques, either by separating isomers of a compound of formula I or by synthesizing individual isomers of a compound of formula I.

Compounds of formula I can exist in unsolvated and solvated forms, including hydrated forms. In general, the solvated forms, with pharmaceutically acceptable solvents such as water, ethanol and the like, are equivalent to the unsolvated forms for purposes of this invention.

A compound of formula I may form pharmaceutically acceptable salts with organic and inorganic acids. Examples of suitable acids for salt formation are hydrochloric, sulfuric, phosphoric, acetic, citric, malonic, salicylic, malic, fumaric, succinic, ascorbic, maleic, methanesulfonic and other mineral and carboxylic acids well known to those skilled in the art. The salts are prepared by contacting the free base forms with a sufficient amount of the desired acid to produce a salt in the conventional manner. The free base forms may be regenerated by treating the salt with a suitable dilute aqueous base solution, such as dilute aqueous sodium hydroxide, potassium carbonate, ammonia or sodium bicarbonate. The free base forms differ from their respective salt forms somewhat in certain physical properties, such as solubility in polar solvents, but the salts are otherwise equivalent to their respective free base forms for purposes of the invention.

In a preferred group of compounds of formula 1, Y is

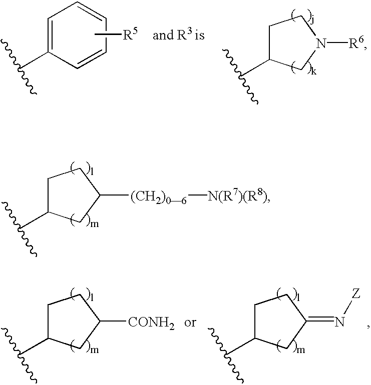

including, in particular, those compounds in which R⁵ is 1-3 substitutents independently selected from the group consisting of H, halogen, haloalkyl and haloalkoxy and the sum of j and k is 1, 2 or 3.

In another preferred group of compounds of formula 1, Y is

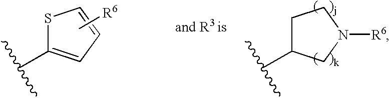

including, in particular, those compounds in which R⁵ and R⁶ each independently is 1 to 3 substituents independently selected from the group consisting of H, halogen, haloalkyl and haloalkoxy and the sum of j and k is 1, 2 or 3.

Compounds of formula I may be produced by processes known to those skilled in the art as shown in the following reaction schemes and in the preparations and examples below.

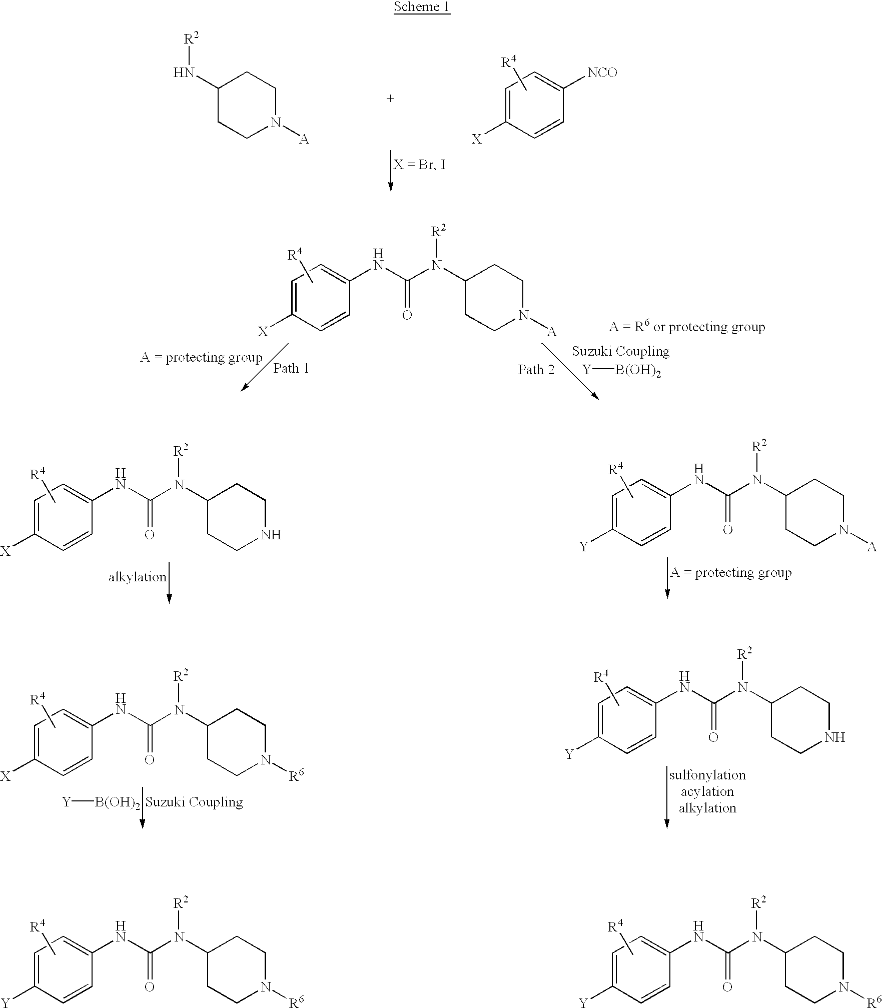

In Scheme 1, a 4-halophenyl isocyanate is condensed with an amino substituted cyclic amine derivative to give a 4-halophenyl urea derivative. Cleavage of the cyclic amine protecting group by methods known to those skilled in the art affords a cyclic amine derivative that can be derivatized, for example by alkylation (Path 1). Coupling of the product with, for example, an arylboronic acid, under palladium catalysis (Suzuki coupling) yields a biaryl urea derivative. Alternatively, the condensation product can be arylated, for example, by use of a Suzuki coupling reaction (Path 2). When A is a protecting group, deprotection affords an amine that can be derivatized by, for example, sulfonylation, acylation or alkylation.

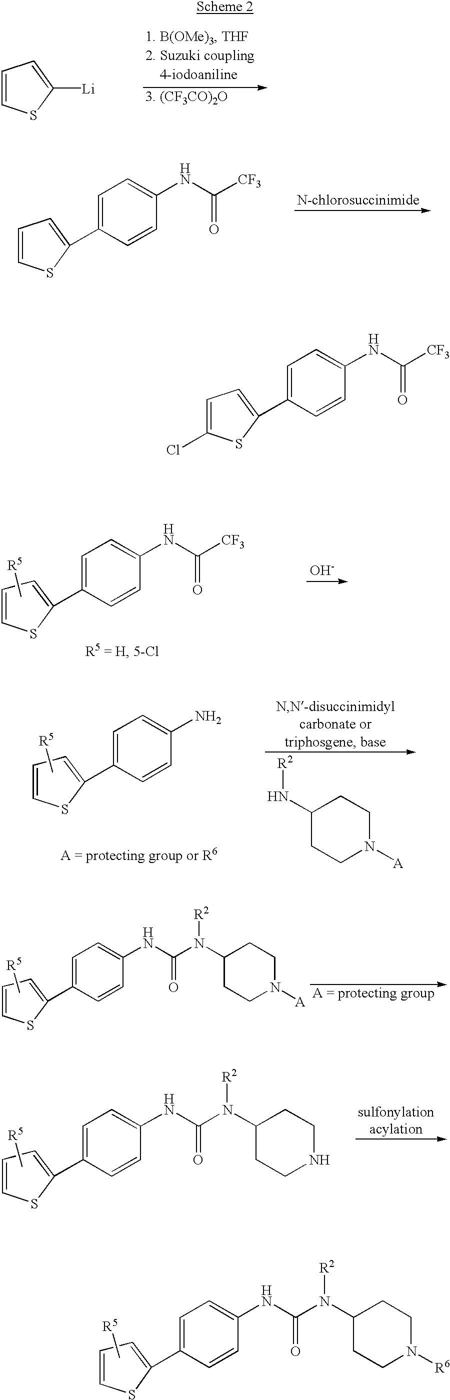

In Scheme 2, reaction of an aryl lithium, for example, 5-thienyl lithium, with trimethylborate and coupling of the resultant boronate with a 4-haloaniline under palladium catalysis yields a biaryl amine derivative. Protection of the amine with, for example, trifluoroacetic anhydride gives a trifluoroacetamide derivative that can be halogenated with an appropriate halogenating agent, for example N-chlorosuccinimide. The protecting group can be cleaved and the resultant amine can be reacted with, for example, N,N′-disuccinimidyl carbonate and an amino substituted cyclic amine derivative, for example an amino piperidine derivative, to give a substituted urea. Cleavage of the piperidine nitrogen protecting group gives an amine that can derivatized, for example, by sulfonylation or acylation.

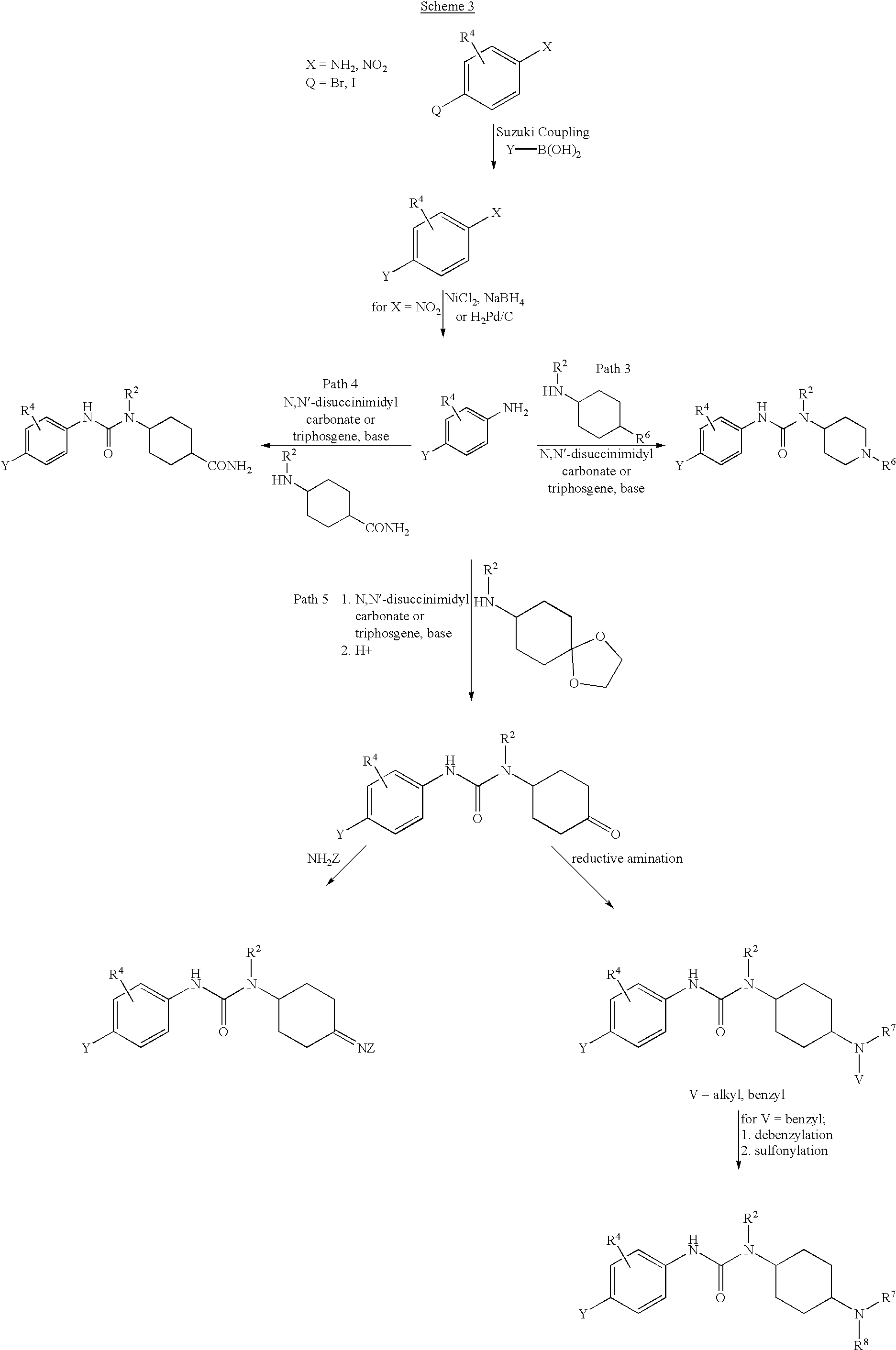

In Scheme 3, a 4-haloaniline or 4-halonitrobenzene derivative is arylated by use of, for example, a Suzuki coupling reaction. When X is a nitro group, the nitro group is subsequently reduced to an amine. The biaryl amine derivative can be converted to an isocyanate derivative, which can be condensed with an amino substituted cyclic amine derivative (Path 3). Alternatively, condensation with an amino substituted cycloalkyl derivative affords cycloalkyl urea derivatives (Paths 4 and 5). An appropriately functionalized cycloalkyl urea derivative can be further functionalized as shown, for example, in Path 5.

The compounds of formula I exhibit selective neuropeptide Y Y5 receptor antagonizing activity, which has been correlated with pharmaceutical activity for treating obesity, eating disorders, such as hyperphagia, and diabetes.

Another aspect of this invention is a method of treating a mammal (e.g., human) having a disease or condition mediated by the neuropeptide Y Y5 receptor by administering a therapeutically effective amount of a compound of Formula I, a prodrug thereof, or a pharmaceutically acceptable salt of said compound or of said prodrug to the mammal.

Another aspect of this invention is directed to a method of treating obesity comprising administering to a mammal in need of such treatment a therapeutically effective amount of a compound of Formula I or a prodrug thereof, or a pharmaceutically acceptable salt of said compound or of said prodrug.

Another aspect of this invention is directed to a method for treating metabolic and eating disorders such as bulimia and anorexia comprising administering to a mammal a therapeutically effective amount of a compound of Formula I, a prodrug thereof, or a pharmaceutically acceptable salt of said compound or of said prodrug.

Another aspect of this invention is directed to a method for treating hyperlipidemia comprising administering to a mammal a therapeutically effective amount of a compound of Formula I, a prodrug thereof, or a pharmaceutically acceptable salt of said compound or of said prodrug.

Another aspect of this invention is directed to a method for treating cellulite and fat accumulation comprising administering to a mammal a therapeutically effective amount of a compound of Formula I, a prodrug thereof, or a pharmaceutically acceptable salt of said compound or of said prodrug.

Another aspect of this invention is directed to a method for treating Type II diabetes comprising administering to a mammal a therapeutically effective amount of a compound of Formula I, a prodrug thereof, or a pharmaceutically acceptable salt of said compound or of said prodrug.

In addition to the “direct” effect of the compounds of this invention on the neuropeptide Y Y5 receptor subtype, there are diseases and conditions that will benefit from the weight loss such as insulin resistance, impaired glucose tolerance, Type II Diabetes, hypertension, hyperlipidemia, cardiovascular disease, gall stones, certain cancers, and sleep apnea.

This invention is also directed to pharmaceutical compositions, which comprise an amount of a compound of Formula I, a prodrug thereof, or a pharmaceutically acceptable salt of said compound or of said prodrug and a pharmaceutically acceptable carrier therefor.

This invention is also directed to pharmaceutical compositions for the treatment of obesity which comprise an obesity treating amount of a compound of Formula, I, a prodrug thereof, or a pharmaceutically acceptable salt of said compound or of said prodrug and a pharmaceutically acceptable carrier therefor.

Compounds of Formula I can be produced by processes known to those skilled in the art using either solution phase or solid phase synthesis as shown in the following reaction schemes, in the preparations and examples below.

The compounds of formula I display pharmacological activity in test procedures designed to demonstrate neuropeptide Y Y5 receptor antagonist activity. The compounds are non-toxic at pharmaceutically therapeutic doses. Following are descriptions of the test procedures.

cAMP Assay

HEK-293 cells expressing the Y5 receptor subtype were maintained in Dulbecco's modified Eagles' media (Gico-BRL) supplemented with 10% FCS (ICN), 1% penicillin-streptomycin and 200 μg/ml Geneticin®(GibcoBRL #11811-031) under a humidified 5% CO₂ atmosphere. Two days prior to assay, cells were released from T-175 tissue culture flasks using cell dissociation solution (1×; non-enzymatic [Sigma #C-5914]) and seeded into 96-well, flat-bottom tissue culture plates at a density of 15,000 to 20,000 cells per well. After approximately 48 hours, the cell monolayers were rinsed with Hank's balanced salt solution (HBSS) then preincubated with approximately 150 μl/well of assay buffer (HBSS supplemented with 4 mM MgCl₂, 10 mM HEPES, 0.2% BSA [HH]) containing 1 mM 3-isobutyl-1-methylxanthine ([IBMX] Sigma #1-587) with or without the antagonist compound of interest at 37° C. After 20 minutes the 1 mM IBMX-HH assay buffer (±antagonist compound) was removed and replaced with assay buffer containing 1.5 μM (CHO cells) or 5 μM (HEK-293 cells) forskolin (Sigma #F-6886) and various concentrations of NPY in the presence or absence of one concentration of the antagonist compound of interest. At the end of 10 minutes, the media were removed and the cell monolayers treated with 75 μl ethanol. The tissue culture plates were agitated on a platform shaker for 15 minutes, after which the plates were transferred to a warm bath in order to evaporate the ethanol. Upon bringing all wells to dryness, the cell residues were resolubilized with 250 μl FlashPlate® assay buffer. The amount of cAMP in each well was quantified using the [¹²⁵I]-cAMP FlashPlate® kit (NEN #SMP-001) and according to the protocol provided by the manufacturer. Data were expressed as either pmol cAMP/ml or as percent of control. All data points were determined in triplicate and EC₅₀'s (nM) were calculated using a nonlinear (sigmoidal) regression equation (GraphPad Prism™). The K_{B} of the antagonist compound was estimated using the following formula:

K_{B}=[B}/(1−{[A′]/[A]})

where

[A] is the EC₅₀ of the agonist (NPY) in the absence of antagonist,

[A′] is the EC₅₀ of the agonist (NPY) in the presence of antagonist,

and [B] is the concentration of the antagonist.

NPY Receptor Binding Assay

Human NPY Y5 receptors were expressed in CHO cells. Binding assays were performed in 50 mM HEPES, pH 7.2, 2.5 mM CaCl₂, 1 mM MgCl₂ and 0.1% BSA containing 5-10 μg of membrane protein and 0.1 nM ¹²⁵L-peptide YY in a total volume of 200 μl. Non-specific binding was determined in the presence of 1 μM NPY. The reaction mixtures were incubated for 90 minutes at room temperature then filtered through Millipore MAFC glass fiber filter plates which had been pre-soaked in 0.5% polyethleneimine. The filters were washed with phosphate-buffered saline, and radioactivity was measured in a Packard TopCount scintillation counter.

For the compounds of this invention, a range of neuropeptide Y5 receptor binding activity from about 0.2 nM to about 500 nM was observed. Compounds of this invention preferably have a binding activity in the range of about 0.2 nM to 250 nM, more preferably about 0.2 to 100 nM, and most preferably about 0.2 to 10 nM.

Yet another aspect of this invention are combinations of a compound of Formula I, a prodrug thereof, or a pharmaceutically acceptable salt of said compound or of said prodrug and other compounds as described below.

Accordingly, another aspect of this invention is a method for treating obesity comprising administering to a mammal (e.g., a female or male human)

a. an amount of a first compound, said first compound being a Formula I compound, a prodrug thereof, or a pharmaceutically acceptable salt of said compound or of said prodrug; and

b. an amount of a second compound, said second compound being an anti-obesity and/or anorectic agent such as a β₃ agonist, a thyromimetic agent, an anoretic agent, or an NPY antagonist wherein the amounts of the first and second compounds result in a therapeutic effect.

This invention is also directed to a pharmaceutical combination composition comprising: a therapeutically effective amount of a composition comprising

a first compound, said first compound being a Formula I compound, a prodrug thereof, or a pharmaceutically acceptable salt of said compound or of said prodrug

a second compound, said second compound being an anti-obesity and/or anorectic agent such as a α₃ agonist, a thyromimetic agent, an anoretic, or an NPY antagonist; and/or optionally a pharmaceutical carrier, vehicle or diluent.

Another aspect of this invention is a kit comprising:

a. an amount of a Formula I compound, a prodrug thereof, or a pharmaceutically acceptable salt of said compound or of said prodrug and a pharmaceutically acceptable carrier, vehicle or diluent in a first unit dosage form;

b. an amount of an anti-obesity and/or anorectic agent such as a β₃ agonist, a thyromimetic agent, an anoretic agent, or an NPY antagonist and a pharmaceutically acceptable carrier, vehicle or diluent in a second unit dosage form; and

c. means for containing said first and second dosage forms wherein the amounts of the first and second compounds result in a therapeutic effect.

Preferred anti-obesity and/or anorectic agents (taken singly or in any combination thereof) in the above combination methods, combination compositions and combination kits are:

phenylpropanolamine, ephedrine, pseudoephedrine, phentermine, a cholecystokinin-A (hereinafter referred to as CCK-A) agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (hereinafter referred to as “leptin”), a leptin analog, a leptin receptor agonist, a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin and ciliary neurotrophic factors such as Axokine.

Another aspect of this invention is a method treating diabetes comprising administering to a mammal (e.g., a female or male human)

a. an amount of a first compound, said first compound being a Formula I compound, a prodrug thereof, or a pharmaceutically acceptable salt of said compound or of said prodrug; and

b. an amount of a second compound, said second compound being an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a PPAR-gamma ligand such as troglitazone, rosaglitazone, pioglitazone or GW-1929, a sulfonylurea, glipazide, glyburide, or chlorpropamide wherein the amounts of the first and second compounds result in a therapeutic effect.

This invention is also directed to a pharmaceutical combination composition comprising: a therapeutically effective amount of a composition comprising

a first compound, said first compound being a Formula I compound, a prodrug thereof, or a pharmaceutically acceptable salt of said compound or of said prodrug;

a second compound, said second compound being an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a PPAR-gamma ligand such as troglitazone, rosaglitazone, pioglitazone, or GW-1929, a sulfonylurea, glipazide, glyburide, or chlorpropamide; and optionally

a pharmaceutical carrier, vehicle or diluent.

Another aspect of this invention is a kit comprising:

a. an amount of a Formula I compound, a prodrug thereof, or a pharmaceutically acceptable salt of said compound or of said prodrug and a pharmaceutically acceptable carrier, vehicle or diluent in a first unit dosage form;

b. an amount of an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a PPAR-gamma ligand such as troglitazone, rosaglitazone, pioglitazone, or GW-1929, a sulfonylurea, glipazide, glyburide, or chlorpropamide and a pharmaceutically acceptable carrier, vehicle or diluent in a second unit dosage form; and

c. means for containing said first and second dosage forms wherein the amounts of the first and second compounds result in a therapeutic effect.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal composition can take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application.

The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in two to four divided doses.

The invention disclosed herein is exemplified by the following preparations and examples which should not be construed to limit the scope of the disclosure.

Alternative mechanistic pathways and analogous structures may be apparent to those skilled in the art.

In the preparations and examples, the following abbreviations are used: room temperature (R.T.), phenyl(Ph),-t-butyloxycarbonyl(-Boc), methylamine (MeNH₂), sodium triacetoxyborohydride (NaBH(O Ac)₃)), ethyl acetate (EtOA_{c}), methanol (MeOH), triethylamine (Et₃ N), ether (Et₂O), tetrahydrofuran (THF), diisopropylethylamine (iPr₂NEt), 1,2-dimethoxyethane (DME), ethanol (EtOH) and preparative thin layer chromatography (PTLC).

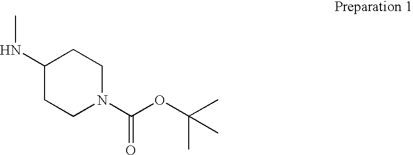

To a mixture of N-t-butoxycarbonyl-4-piperidone (10.0 g, 50 mmol) and aqueous methylamine (40% w/w, 10 ml) in 1,2-dichloroethane (125 ml) was added NaBH(OAc)₃ (16.0 g, 75 mmol). The reaction mixture was stirred overnight, then 1 M NaOH (250 ml) was added and the whole was extracted with ether (700 ml). The organic layer was washed with sat'd NaCl, dried (MgSO₄), filtered, and concentrated to give the product (10.5 g, 97%) as an oil. ¹H NMR (CDCl₃, 400 MHz) δ4.09 (2H, m), 2.86 (2H, m), 2.55 (1H, m), 2.50 (3H, s), 1.90 (2H, m), 1.51 (9H, s), 1.30 (2H, m).

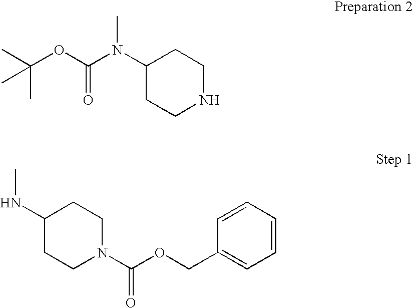

To a mixture of N-benzyloxycarbonyl-4-piperidone (10.70 g, 43.1 mmol) and aq. 40% MeNH₂ (6.67 g, 85.8 mmol) in CH₂Cl₂ (200 ml) at R.T. was added NaBH(OAc)₃ (27.25 g, 128.6 mmol). The reaction mixture was stirred at R.T. for 3 h then poured into sat'd NaHCO₃ and extracted with CH₂Cl₂ (3×200 ml). The combined organic layers were dried (Na₂SO₄), filtered and concentrated to give the product (10.63 g, 100%) that was used without further purification. ¹H NMR (CDCl₃, 400 MHz) δ7.34 (5H, m), 5.12 (2H, s), 4.19 (2H, b), 2.87 (2H, b), 2.72 (1H, m), 2.49 (2H, b), 1.42 (2H, m). MS m/e 249 (M+H).

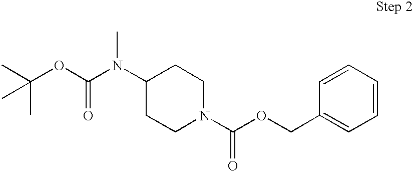

To the product of Step 1 (10.63 g, 42.9 mmol) in anhydrous CH₂Cl₂ (200 ml) at R.T. was added di-tert-butyl dicarbonate (11.30 g, 51.8 mmol) in portions. The reaction mixture was allowed to stir at R.T. for 5 h then poured into 1 N NaOH (50 ml)/CH₃OH (10 ml). The mixture was stirred for 15 min. and extracted with CH₂Cl₂ (3×200 ml). The combined organic layers were dried (Na₂SO₄), filtered, and concentrated. The residue was subjected to column chromatography (gradient 1:10 to 1:4 EtOAc/hexane) to give the product (13.00 g, 87%). ¹H NMR (CDCl₃, 400 MHz) δ7.33 (5H, m), 5.10 (2H, s), 4.19 (3H, m), 2.87 (2H, b), 2.68 (3H, s), 1.60 (4H, m), 1.44 (9H, s). MS m/e 349 (M+H).

Step 3

A mixture of the product of Step 2 (12.90 g, 37.0 mmol) and 10% Pd/C (1.29 g) in MeOH (300 ml) was stirred under an H₂ atmosphere. After 16 h the reaction mixture was filtered through celite and the filter pad was washed with MeOH. The combined filtrate and washings were concentrated to afford the product (7.8 0 g, 98.3%). ¹H NMR (CDCl₃, 40MHz δ4.19 (2H, b), 3.15 (2H, b), 2.74 (3H, s), 2.66 (2H, m), 1.63 (4H, m), 1.46 (9H, s). MS m/e 215 (M+H).

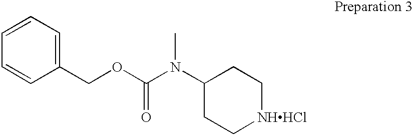

To a stirred solution of Preparation 1 (21.0 g, 83.7 mmol) and Et₃N (35 ml, 252 mmol) in CH₂Cl₂ (300 ml) was added benzyl chloroformate (18 ml, 126 mmol) dropwise. After 5 h, sat'd NH₄Cl (200 ml) was added, and the organic layer was washed with H₂O (150 ml) and sat'd NaCl (150 ml), dried (MgSO₄), filtered and concentrated. To the residue (32 g) was added 4N HCl in 1,4-dioxane (300 ml), and the mixture was stirred for 4 h. The reaction mixture was concentrated, acetone was added, and the reaction mixture was again concentrated. The solid residue was dissolved in MeOH (40 ml) and Et₂O was added. The resultant precipitate was collected, washed with Et₂O, and dried to give the product as a white solid (20.2 g, 85%). MS m/e 249 (M+H, free base).

### EXAMPLE 1

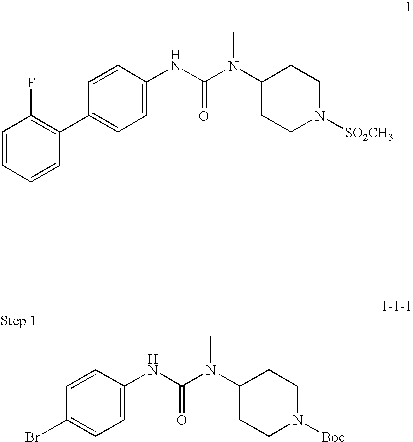

To a solution of Preparation 1 (7.0 g, 33 mmol) in CH₂Cl₂ (200 ml) was added 4-bromophenyl isocyanate (6.8 g, 35 mmol). The reaction mixture was stirred for 16 h, then H₂O (200 ml) was added, and the organic layer was dried (MgSO₄), filtered and evaporated. The residue was triturated with hexanes to give a white solid (11.0 g, 81%). MS (FAB) m/e 411 (M+H)⁺.

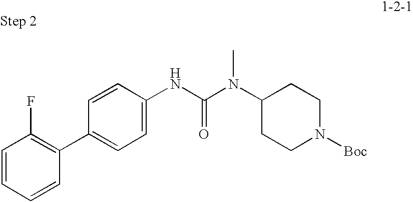

To a solution of the product of Step 1 (400 mg, 0.97 mmol) and Pd(dppf)Cl₂.CH₂Cl₂ (200 mg, 0.24 mmol) in toluene (10 ml) was added 2-fluorophenylboronic acid (250 mg, 1.43 mmol), Cs₂CO₃ (350 mg, 1.1 mmol), and H₂O (0.3 ml). The reaction mixture was heated in a 90° C. oil bath under N₂ for 1 h, then allowed to cool. The reaction mixture was partitioned between EtOAc (100 ml) and H₂O (50 ml). The organic layer was dried (MgSO₄), filtered and evaporated. Flash chromatography (3:7 acetone/hexane) of the residue afforded the product (400 mg, 97%). HRMS calc. for C₂₄H₃₁FN₃O₃ (M+H) 428.2349. Found 428.2343.

Coupling of the product of Step 1 with the appropriate boronic acid by essentially the same procedure gave:

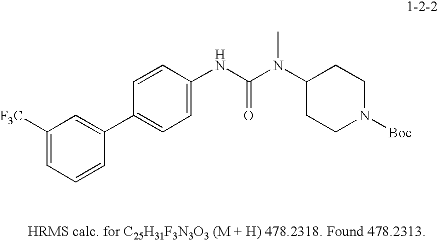

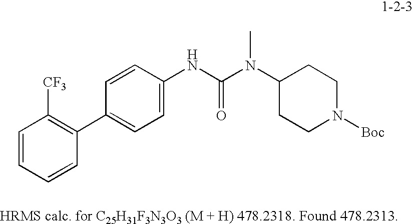

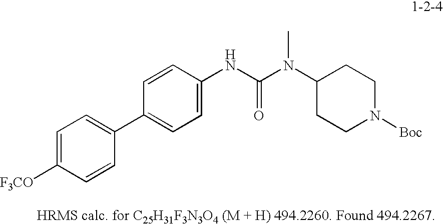

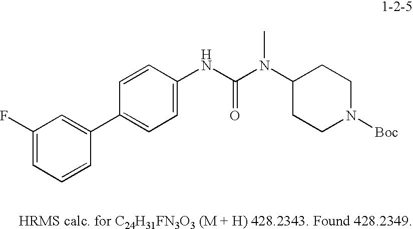

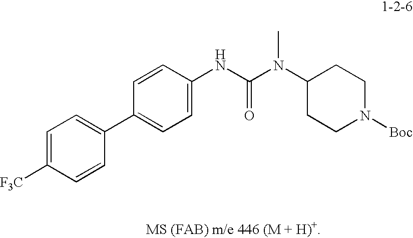

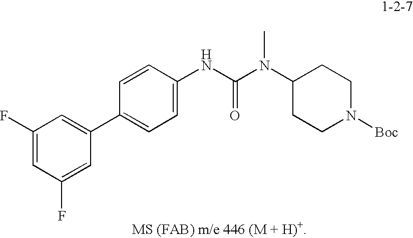

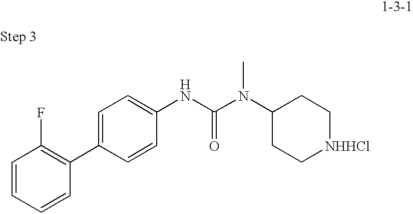

To a solution of the product of Step 2 (100 mg, 0.23 mmol) in CH₂Cl₂ (5 ml) was added 4 M HCl in 1,4-dioxane (3 ml). After 16 h, the reaction mixture was concentrated. The residue was triturated with ether and the solid was collected, washed with ether, and air-dried to give the product (80 mg, 96%). HRMS calc. for C₁₉H₂₃FN₃O(M+H) 328.1825. Found 328.1823.

Treatment of the other products from Step 2 by essentially the same procedure gave:

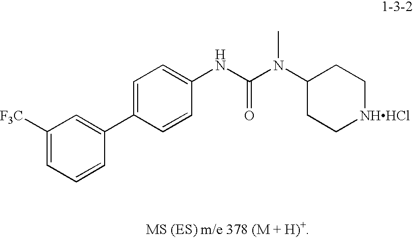

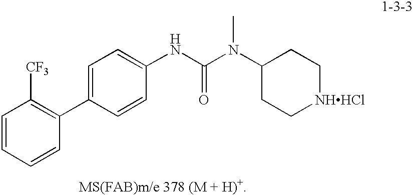

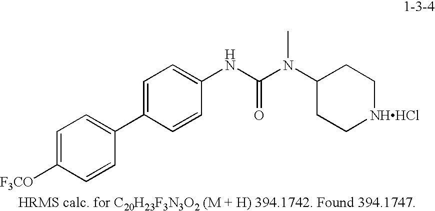

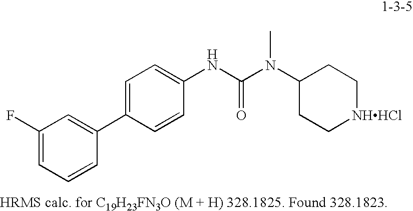

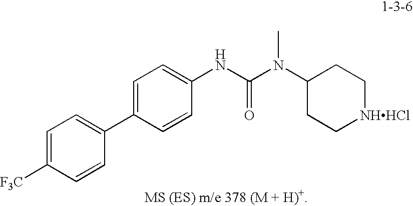

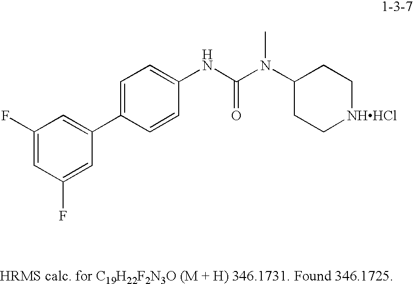

Step 4

To a stirred solution of the product of Step 3 (20 mg, 0.055 mmol) and triethylamine (0.1 ml, 0.7 mmol) in CH₂Cl₂ (10 ml) was added methanesulfonyl chloride (0.1 ml, 0.1 mmol). After 16 h the reaction mixture was concentrated and the residue was subjected to PTLC (1:2 acetone/hexanes) to give a white solid (15 mg, 67%). HRMS calc. for C₂₀H₂₅FN₃O₃S (M+H) 406.1601. Found 406.1599.

The following examples were prepared from the appropriate starting amine and sulfonyl chloride.

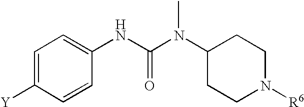

| Y | R⁶ | MS (M + H) | Example |
|---|---|---|---|
| 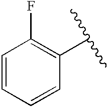 | —SO₂CF₃ | 460 | 1A |
| 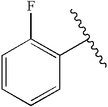 | —SO₂CH(CH₃)₂ | 434 | 1B |
| 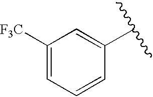 | —SO₂CH₃ | 456 | 1C |
| 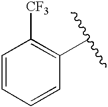 | —SO₂CH₃ | 456 | 1D |
| 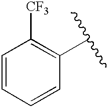 | —SO₂CH(CH₃)₂ | 484 | 1E |
| 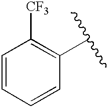 | —SO₂CF₃ | 510 | 1F |
| 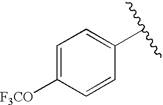 | —SO₂CH₃ | 472 | 1G |
| 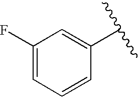 | —SO₂CH₃ | 406 | 1H |
| 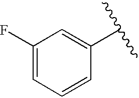 | —SO₂CF₃ | 460 | 1I |
| 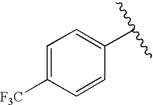 | —SO₂CH₃ | 456 | 1J |
| 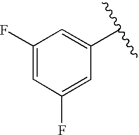 | —SO₂CH₃ | 424 | 1K |

### EXAMPLE 2

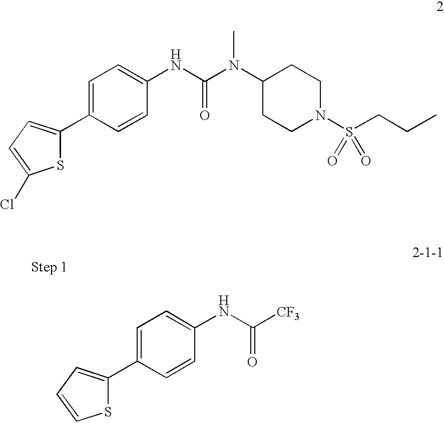

A stirred solution of 1M 1-thienyllithium in THF (40 ml, 40 mmol) was cooled in a dry-ice/acetone bath under N₂. Triethylborate (8.5 ml, 50 mmol) was added, and the reaction mixture was allowed to warm to R.T. After 20 min., 4-iodoaniline (6.6 g, 30 mmol), Na₂CO₃ (4.5 g), H₂O (20 ml), and Pd(dppf)Cl₂.CH₂Cl₂ (750 mg, 0.9 mmol were added. The reaction mixture was stirred under N2 until the exotherm was complete, then partitioned between Et₂O and H₂O. The Et₂O layer was washed with 1N NaOH, dried (Na₂CO₃), and filtered through a pad of silica gel, eluting with Et₂O. The resultant brown solid was dissolved in CH₂Cl₂ (100 ml) and a solution of trifluoroacetic anhydride (8 ml, 57 mmol) in CH₂Cl₂ (100 ml) was added in portions with stirring. To the resultant suspension was added CH₂Cl₂ (450 ml) and the reaction mixture was stirred for 20 min. Water (200 ml) was added, followed by NaHCO₃ (7 g) in portions until CO₂ evolution ceased. The organic layer was stirred with MgSO₄ and DARCO, then filtered and concentrated to give a solid. The solid was dissolved in CH₂Cl₂ (50 ml) and to the stirred solution was added hexanes (100 ml). The solid was collected, washed with hexanes and dried to give the product (6.12 g, 75%). M.p. 213-216° C. Calcd for C₁₂H₈F₃NOS: C, 53.14; H, 2.58; N, 5.17. Found: C, 53.06; H 2.85; N, 4.90%.

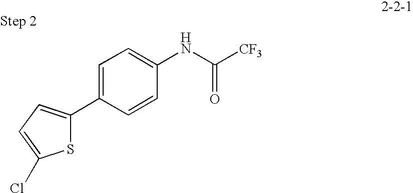

To a solution of the product of Step 1 (19.0 g, 70 mmol) in DMF (150 ml) was added N-chlorosuccinimide (10.1 g, 76 mmol) and trifluoroacetic acid (1.5 ml), and the reaction mixture was stirred under N₂ for 2 days. Water (500 ml) was added and the resultant solid was collected, washed with water and dried to give the product (20.6 g, 96%). M.P. 198-200° C. Calcd for C₁₂H₇C/F₃NOS: C, 47.12; H, 2.29; N, 4.58. Found: C, 47.19; H, 2.15; N, 4.47%.

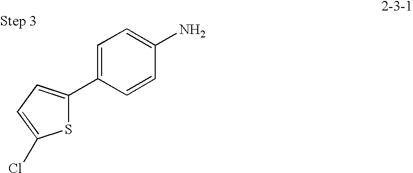

A mixture of the product of Step 2 (15.0 g, 49.1 mmol) and sodium hydroxide (19.6 g, 490 mmol) in MeOH (400 ml) and water (150 ml) was stirred at R.T. overnight. The mixture was concentrated in vacuo and the residue was partitioned between EtOAc and water. The organic layer was washed with water, brine, dried, and concentrated. The residue was purified by flash column (1:3 acetone/hexanes) to give the product (10.14 g, 98%). ¹H-NMR (CDCl₃, 400 MHz) δ7.32 (2H, m), 6.90 (1H, d, J=4.8 Hz), 6.83 (1H, d, J=4.8 Hz), 6.67 (2H, m), 3.76 (2H, b).

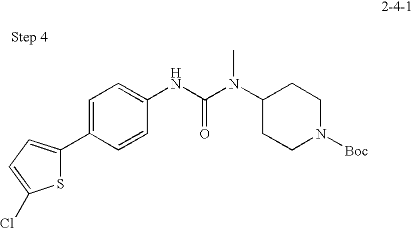

To a stirred, ice-cold solution of the product of Step 3 (2.0 g, 9.5 mmol) in THF (100 ml) was added pyridine (2.3 ml, 28 mmol) and N,N′-disuccinimidyl carbonate (2.44 g, 9.5 mmol). The reaction mixture was stirred at ice-bath temp. for 1.5 h, then Preparation 1 (2.04 g, 9.5 mmol) was added, and the reaction mixture was allowed to warm to R.T. After 16 h, the reaction mixture was concentrated, the residue was dissolved in EtOAc (200 ml) and washed with 2N HCl, sat'd NaHCO₃ and sat'd NaCl. The organic layer was dried (Na₂SO₄), filtered, and evaporated to afford the product (4.21 g, 98%) that was used directly in Step 5. HRMS calc. for C₂₂H₂₉C/N₃O₃S (M+H) 450.1618. Found 450.1623.

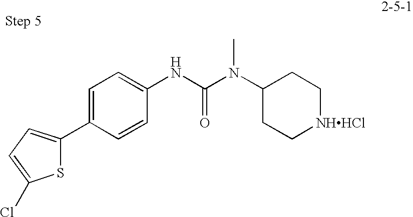

Reaction of the product of Step 4 (4.11 g, 9.13 mmol) with HCl by the procedure of Example 1, Step 3 afforded the product (3.71 g) that was used directly in Step 6. HRMS calc. for C₁₇H₂₁C/N₃OS (M+H) 350.1094. Found 350.1100.

### Step 6

To a suspension of the product of Step 5 (50 mg, 0.13 mmol) in CH₂Cl₂ (3 ml) was added Et₃N (39 mg, 0.39 mmol) followed by n-propylsulfonyl chloride (20 mg, 0.14 mmol). The reaction mixture was stirred for 16 h. EtOAc (10 ml) was added and the mixture was washed with 2N HCl, sat'd NaHCO₃ and sat'd NaCl, dried (MgSO₄), filtered and concentrated. The residue was subjected to PTLC (3:97 MeOH/CH₂Cl₂) to give the product (37 mg, 62%). HRMS calc. for C₂₀H₂₇C/N₃O₃S₂ (M+H) 456.1182. Found 456.1179.

Reaction of the product of Step 5, 2-5-1, with the appropriate sulfonyl chloride in the presence of Et₃N gave the following examples.

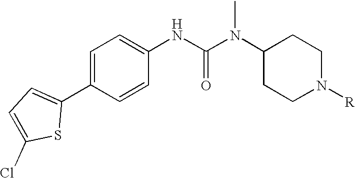

| R⁶ | MS (M + H)+ | Example |
|---|---|---|
| -SO₂CH₃ | 428 | 2A |
| —SO₂CH₂CH₃ | 442 | 2B |
| —SO₂CH(CH₃)₂ | 456 | 2C |
| —SO₂CF₃ | 482 | 2D |
| —SO₂CH₂CF₃ | 496 | 2E |

### EXAMPLE 3

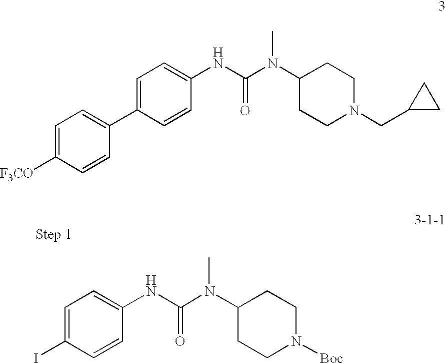

Using the procedure of Example 1, Step 1, Preparation 1 (2.3 g, 107 mmol) was reacted with 4-iodophenyl isocyanate (2.6 g, 107 mmol). Purification by flash chromatography (2:98 MeOH/CH₂Cl₂) afforded a white solid.

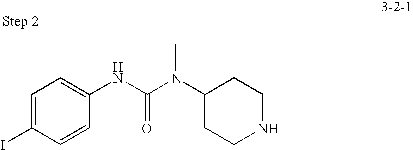

A mixture of the product of Step 1 (3.0 g, 6.7 mmol), 4M HCl in 1,4-dioxane (15 ml) and THF (15 ml) was stirred at ambient temp. for 5 h. The reaction mixture was concentrated to dryness, and H₂O (100 ml) and 3M NaOH (20 ml) was added to the residue. The whole was extracted with CH₂Cl₂ (3×100 ml). The combined organic extracts were dried (MgSO₄), filtered and evaporated. Flash chromatography (2:98 MeOH/CH₂Cl₂ then 10:90 (2M NH₃ in MeOH)/CH₂Cl₂) gave a white solid (2.4 g, 100%). HRMS calc. for C₁₃H₁₉IN₃O(M+H) 360.0573. Found 360.0576.

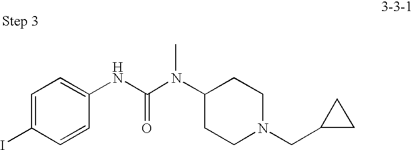

To a stirred ice-cold mixture of the product of Step 2 (2.4 g, 6.7 mmol) and cyclopropane carboxaldehyde (0.8 ml, 11 mmol) in CH₂Cl₂ (20 ml) was added NaBH(OAc)₃ (1.83 g, 10.8 mmol). The reaction mixture was allowed to warm to room temp. and stirred overnight. The reaction mixture was cooled in ice and 3M NaOH (5 ml) was added. After 0.5 h the mixture was extracted with CH₂Cl₂ (3×100 ml), dried (MgSO₄), filtered and evaporated. The residue was triturated with CH₂Cl₂/hexanes (1:10) to afford a white solid (2.4 g, 87%). HRMS calc. for C₁₇H₂₅IN₃O(M+H) 414.1038. Found 414.1042.

Step 4

A vessel charged with the product of Step 3 (200 mg, 0.48 mmol), 4-trifluoromethoxybenzeneboronic acid (250 mg, 1.21 mmol), tris(dibenzylideneacetone)dipalladium (0) (50 mg, 0.05 mmol), CsCO₃ (0.8 g, 2.5 mmol) and toluene (10 ml) was refluxed under N₂ for 3 h. The reaction mixture was allowed to cool, then EtOAc (50 ml) and H₂O (25 ml) were added. Solids were removed by filtration and the EtOAc layer was dried (Na₂SO₄), filtered, and evaporated. The residue was subjected to PTLC (3:7 acetone/hexanes then 10:90 (2M NH₃ in MeOH)/CH₂Cl₂) to give a pale yellow solid (50 mg, 23%). HRMS calc. for C₂₄H₂₉F₃N₃O₂ (M+H) 448.2212. Found 448.2215.

Using appropriate starting materials and essentially the same procedure, the following compounds were prepared:

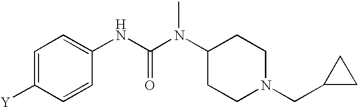

| Y | MS (M + H)⁺ | Example |
|---|---|---|
| 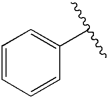 | 364.1 | 3A |
| 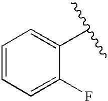 | 382 | 3B |
| 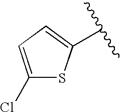 | 404 | 3C |
| 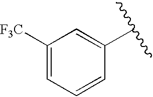 | 423 | 3D |
| 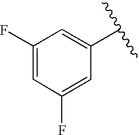 | 400 | 3E |
| 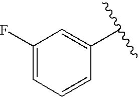 | 382 | 3F |

### EXAMPLE 4

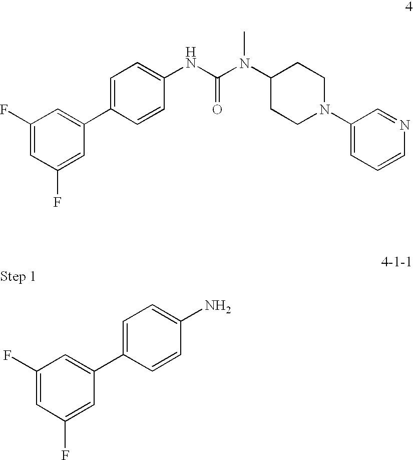

To an N₂-purged mixture of 4-bromonitrobenzene (20.0 g, 99.0 mmol), 3,5-difluorophenylboronic acid (23.4 g, 148 mmol) and Cs₂CO₃ (38.7 g, 119 mmol) in toluene (600 ml) and H₂O (30 ml) was added Pd(dppf)Cl₂.CH₂Cl₂ (4.04 g, 4.95 mmol). The reaction mixture was heated at 90° C. for 2 h, allowed to cool to R.T., then filtered through celite. The whole was extracted with EtOAc (3×500 ml). The combined organic layers were dried (Na₂SO₄), filtered, and concentrated to give a solid. To a vigorously stirred ice-cold mixture of the solid in CH₃OH (1 L) and NiCl₂.6H₂O (61.0 g, 257 mmol) was added NaBH₄ (14 g, 370 mmol) in portions. After the addition was complete, the reaction mixture was poured into H₂O (100 ml), then filtered through celite and extracted with EtOAc (3×500 ml). The combined organic layers were dried (Na₂SO₄), filtered, and concentrated. The residue was dissolved in EtOAc, and 1N HCl/Et₂O (300 ml) was added. The precipitate was washed with hexane, air-dried, and dissolved in H₂O. The solution was neutralized by addition of 1N NaOH, then extracted with CH₂Cl₂ (3×1 L). The combined organic layers were dried (Na₂SO₄), filtered, and concentrated to give the product (19.0 g, 94%). ¹H NMR (CDCl₃, 400 MHz) δ7.38 (2H, m), 7.06 (2H, m), 6.75 (2H, m), 6.72 (1H, m), 3.81 (s, 2H). MS m/e 206 (M+H).

Using the appropriate substituted phenylboronic acid starting material and essentially the same procedure, the following compounds were prepared:

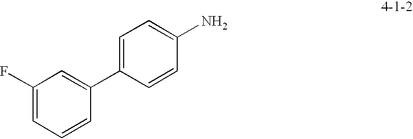

¹H NMR (CDCl₃, 400 MHz) δ7.41-7.21 (5H, m), 7.33 (1H, m), 6.76 (2H, m), 3.76 (2H, b).

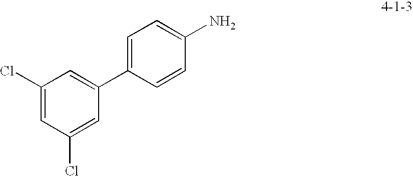

¹H NMR (CDCl₃, 400 MHz) δ7.39 (2H, m), 7.24 (3H, m), 6.76 (2H, m), 3.80 (2H, b).

Additional arylamines were prepared from 4-iodoaniline according to the following procedure.

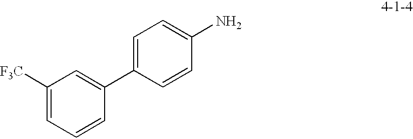

A mixture of 4-iodoaniline (1.00 g, 4.57 mmol), 3-trifluoromethylphenylboronic acid (1.30 g, 6.85 mmol) and Cs₂CO₃ (1.64 g, 5.02 mmol) in toluene (50 ml) and H₂O (3 ml) was purged with N₂ for 5 min. To the reaction mixture was added Pd(dppf)Cl₂.CH₂Cl₂ (746 mg, 0.91 mmol). The reaction mixture was heated at 90° C. for 5 h, then allowed to cool to R.T. and poured into cold water. The whole was extracted with CH₂Cl₂ (3×100 ml). The combined organic layers were dried (Na₂SO₄), filtered and evaporated. Purification of the residue by PTLC (EtOAc/hexane 1:2) gave the product (216 mg, 20%). ¹H NMR (CDCl₃, 400 MHz) δ7.77 (1H, m), 7.70 (1H, m), 7.51 (2H, m), 7.42 (2H, m), 6.78 (2H, m), 3.65 (2H, b).

Using the appropriate substituted phenylboronic acid starting material and essentially the same procedure, the following compounds were prepared.

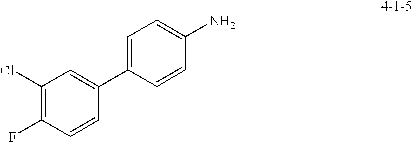

¹H NMR (CDCl₃, 400 MHz) δ7.54 (1H, m), 7.34 (3H, m), 7.15 (1H, t, J=8.8 Hz), 6.75 (2H, m), 3.76 (2H, b).

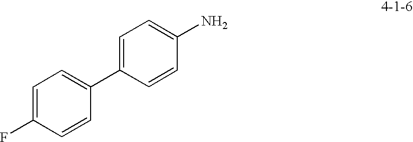

¹H NMR (CDCl₃, 400 MHz) δ7.48 (2H, m), 7.35 (2H, d, J=6.4 Hz), 7.08 (2H, t, J=6.4 Hz), 6.76 (2H, d, J=6.4 Hz), 3.73 (2H, b). MS m/e 188 (M+H).

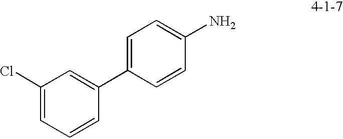

¹H NMR (CDCl₃, 400 MHz) δ7.51 (1H, m), 7.41 (3H, m), 7.32 (1H, m), 7.23 (1H, m), 6.75 (2H, m), 3.78 (2H, b). MS m/e 204 (M+H).

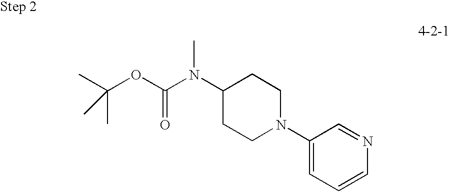

A stream of N₂ was passed through a mixture of the product of Preparation 2 (2.00 g, 9.33 mmol), 3-bromopyridine (2.95 g, 18.7 mmol) and 2-(di-tert-butylphosphino)biphenyl (0.139 g, 0.467 mmol) and NaOtBu (1.80 g, 18.7 mmol) in anhydrous toluene (10 ml). Pd(OAc)₂ (0.105 g, 0.467 mmol) was added and the reaction mixture was stirred at 110° C. for 24 h. The reaction mixture was allowed to cool to R.T. and poured into cold H₂O. The whole was extracted with CH₂Cl₂ (3×50 ml) and the combined organic layers were dried (Na₂SO₄), filtered, and concentrated. Purification of the residue by PTLC (1:20 CH₃OH/CH₂Cl₂) gave the product (1.47 g, 54%). ¹H NMR (CDCl₃, 400 MHz) δ8.29 (1H, s),8.07 (1H, b), 7.17 (2H, m), 4.2 (1H, b), 3.74 (2H, m), 2.82 (2H, m), 2.74 (3H, s), 1.70 (4H, m), 1.45 (9H, s). MS m/e 292 (M+H).

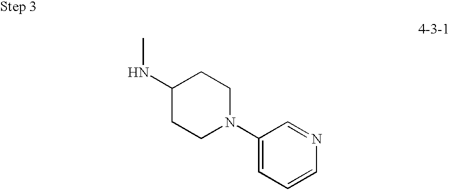

To the product of Step 2 (1.47 g, 5.05 mmol) was added 4M HCl/1,4-dioxane (20 ml). The reaction mixture was stirred at R.T. for 1.5 h and concentrated to afford the product in quantitative yield. ¹H NMR (CD₃OD, 400 MHz) δ8.46 (1H, s), 8.14 (2H, m), 7.86 (1H, s), 4.13 (2H, m), 3.40 (IH, b), 3.16 (2H, b), 2.75 (3H, s), 2.26 (2H, M), 1.76 (2H, m). MS m/e 192 (M+H).

Step 4

To a mixture of the product of Step 1 (4-1-1) (0.100 g, 0.487 mmol) and iPr₂NEt (0.43 ml, 2.44 mmol) in anhydrous toluene (10 ml) was added triphosgene (0.051 g, 0.171 mmol). The mixture was stirred at 120 ° C. for 2 h, then allowed to cool to R.T., and the product of Step 3 (4-3-1) (0.133 g, 0.585 mmol) was added. The reaction mixture was stirred at R.T. for 16 h, then poured into cold H₂O and extracted with CH₂Cl₂ (3×20 ml). The combined organic layers were dried (Na₂SO₄), filtered, and concentrated. The residue was purified by PTLC (1:20 CH₃OH/CH₂Cl₂) to give the product (0.114 g, 56%). ¹H NMR (CDCl₃, 400 MHz) δ8.33 (1H, d, J=2.4 Hz), 8.09 (1H, m), 7.49 (4H, m), 7.17 (2H, m), 7.06 (2H, m), 6.74 (1H, m), 6.51 (1H, s) 4.49 (1H, m), 3.77 (2H, m), 2.93 (3H, s), 2.91 (2H, m), 1.85 (4H, m). MS m/e 423 (M+H).

### EXAMPLE 5

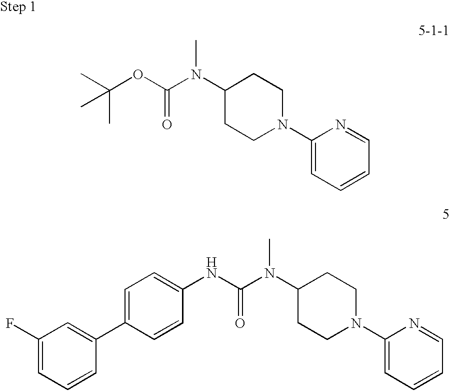

The product 5-1-1 was prepared in 57% yield from 2-bromopyridine and Preparation 2 by the procedure of Example 4, Step 2, except that 2-(di-tert-butylphosphino)biphenyl was replaced by 1,3-bis(diphenylphosphino)propane, and a reaction temperature of 80° C. instead of 110° C. was used. MS m/e 292 (M+H).

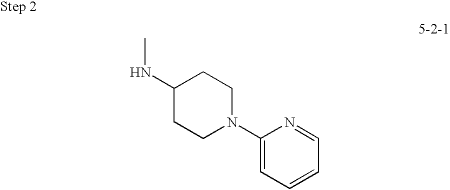

Treatment of the product of Step 1 with 4 N HCl/dioxane by the procedure of Example 4, Step 3 gave the product. MS m/e 192 (M+H).

Step 3

To a stirred ice-cold mixture of 4-1-2 (0.063 g, 0.339 mmol) and pyridine (0.14 ml, 1.69 mmol) in anhydrous THF (10 ml) was added N,N′-disuccinimidyl carbonate (0.087 g, 0.339 mmol). The reaction was stirred in an ice-bath for 25 min. then the product of Step 2, 5-2-1(0.100 g, 0.508 mmol), was added. The reaction was allowed to warm to R.T., stirred for 16 h, then poured into cold H₂O (20 ml). The whole was extracted with CH₂Cl₂ (3×20 ml), the combined organic layers were dried (Na₂SO₄), filtered, and concentrated. The residue was subjected to PTLC (1:20 CH₃OH/CH₂Cl₂) to give the product (0.080 g, 58%). ¹H NMR (CDCl₃, 400 MHz) δ8.19 (1H, m), 7.52 (5H, m), 7.37 (2H, m), 7.27 (1H, m), 6.99 (1H, m), 6.69 (1H, d, 6.62 (1H, m), 6.45 (1H, s), 4.56 (1H, m), 4.42 (2H, m), 2.92 (2H, m), 2.88 (3H, s), 1.78 (4H, m). MS m/e405 (M+H).

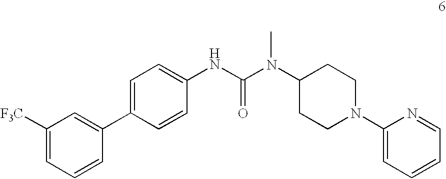

Reaction of 4-1-4, N,N′-disuccinimidyl carbonate and 5-2-1 by the procedure of Example 5, Step 3 afforded the product. MS m/e 455 (M+H).

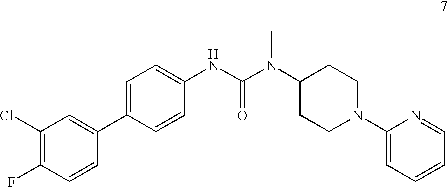

Reaction of 4-1-5, N,N′-disuccinimidyl carbonate and 5-2-1 by the procedure of Example 5, Step 3 afforded the product. MS m/e 473 (M+H).

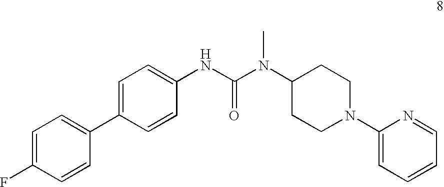

Reaction of 4-1-6, N,N′-disuccinimidyl carbonate and 5-2-1 by the procedure of Example 5, Step 3 afforded the product. MS m/e 405 (M+H).

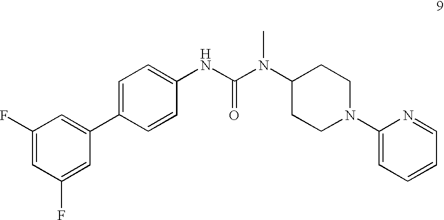

Reaction of 4-1-1, N,N′-disuccinimidyl carbonate and 5-2-1 by the procedure of Example 5, Step 3 afforded the product. MS m/e 423 (M+H).

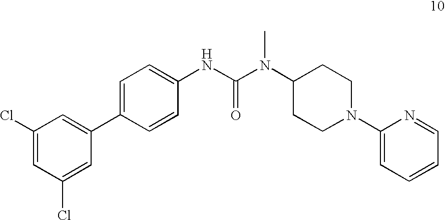

Reaction of 4-1-3, triphosgene and 5-2-1 by the procedure of Example 4, Step 4 afforded the product. MS m/e 455 (M+H).

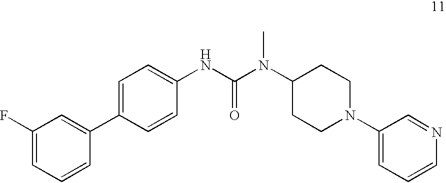

Reaction of 4-1-2, triphosgene and 4-3-1 by the procedure of Example 4, Step 4 afforded the product. MS m/e 405 (M+H).

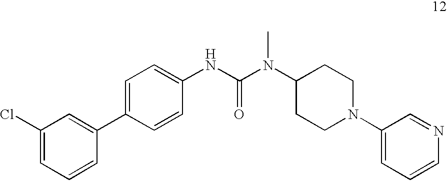

Reaction of 4-1-7, triphosgene and 4-3-1 by the procedure of Example 4, Step 4 afforded the product. MS m/e 421 (M+H).

### EXAMPLE 13

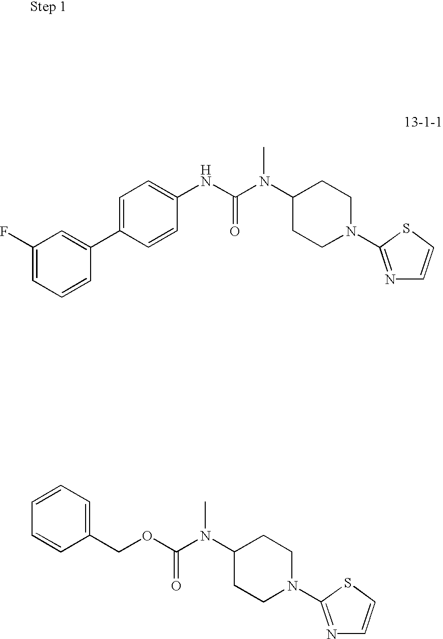

A mixture of Preparation 3 (2.75 g, 9.7 mmol), 2-bromothiazole (1.98 g, 12.1 mmol), and K₂CO₃ (3.5 g, 25 mmol) in DMF (40 ml) was heated at 160° C. for 20 h. The reaction mixture was concentrated and partitioned between CH₂Cl₂ and H₂O. The organic layer was washed with sat'd NaCl, dried (MgSO₄), filtered and concentrated. Flash chromatography (gradient; CH₂Cl₂ to 2:98 MeOH/CH₂Cl₂) gave the product (2.0 g, 62%). MS m/e 332.1 (M+H).

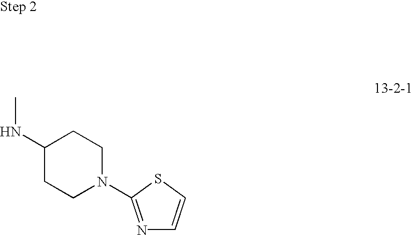

The product of Step 1 (2.0 g, 6.0 mmol) and 33% HBr in AcOH (40 ml) was stirred at R.T. for 2 h. The reaction mixture was evaporated and the residue was partitioned between 1 N NaOH and CH₂Cl₂. The organic layer was washed with sat'd NaCl, dried (MgSO₄), filtered and evaporated. Flash chromatography (gradient; 2:98 (2M NH₃ in MeOH)/CH₂Cl₂ to 15:85 (2M NH₃ in MeOH)/CH₂Cl₂) gave the product (0.94 g, 79%) as a yellow solid. ¹H NMR (CDCl₃, 400 MHz) δ7.04 (1H, d, J=4 Hz), 6.52 (1H, d, J=4 Hz), 3.96 (2H, m), 3.17 (1H, m), 2.99 (2H, m), 2.59 (3H, s), 2.16 (2H, m), 1.68 (2H, m). MS m/e 198 (M+H).

Step 3

Reaction of 4-1-2, triphosgene and 13-2-1 by the procedure of Example 4, Step 4 afforded the product. MS m/e 411 (M+H).

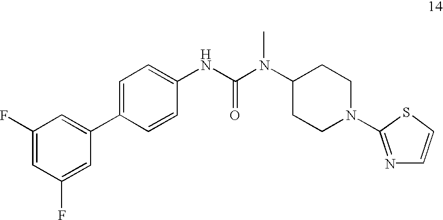

Reaction of 4-1-1, triphosgene and 13-2-1 by the procedure of Example 4, Step 4 afforded the product. MS m/e 429 (M+H).

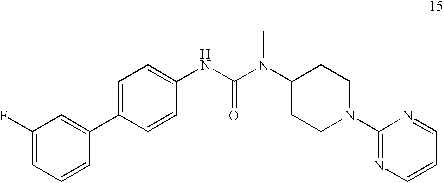

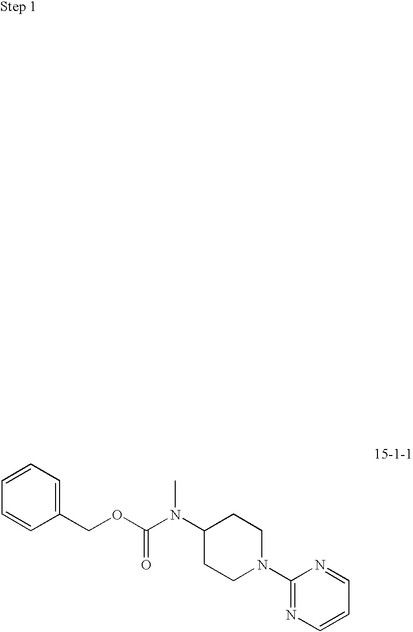

An N₂-purged mixture of 2-bromopyrimidine (400 mg, 2.52 mmol), Preparation 3 (510 mg, 1.79 mmol), Pd(OAc)₂ (18 mg, 0.08 mmol), sodium tert-butoxide (516 mg, 5.37 mmol), and (1,3-bis-diphenylphosphino)propane (29 mg, 0.07 mmol) in toluene (6 ml) was stirred at 70° C. in a sealed vessel for 16 h. The reaction mixture was allowed to cool to R.T., and 1N NaOH (20 ml) was added. The whole was extracted with CH₂Cl₂ (3×20 ml), and the combined CH₂Cl₂ extracts were dried (MgSO₄), filtered, and evaporated. The residue was subjected to PTLC (2:98 MeOH/CH₂Cl₂) to give the product (464 mg, 79%). MS m/e 327 (M+H).

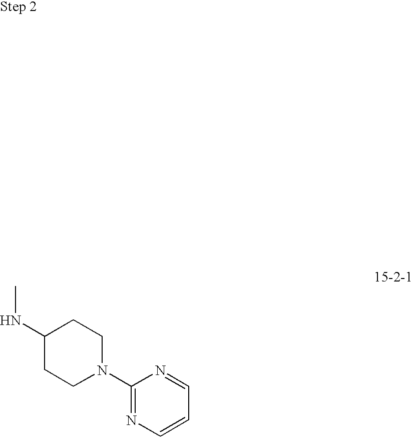

The product of Step 1 (464 mg, 1.43 mmol) and 10% Pd/C (59 mg) in EtOH (20 ml) was stirred under 1 atm. of H₂ for 16 h. The catalyst was removed by filtration through celite and the filter pad was washed with EtOH. The combined filtrate and washings were evaporated. The residue was subjected to PTLC (5:95 (2M NH₃ in MeOH)/CH₂Cl₂) to give the product (464 mg, 79%). ¹H NMR (CDCl₃, 400 MHz) δ8.28 (2H, m), 6.44 (1H, m), 4.66 (2H, m), 2.99 (2H, m), 2.65 (1H, m), 2.47 (3H, s), 1.96 (2H, m), 1.33 (2H, m). MS m/e 193 (M+H).

Step 3

Reaction of the product of Step 2 (15-2-1) with 4-1-2 with triphosgene by the procedure of Example 4, Step 4 gave the product. MS (m/e) 406 (M+H).

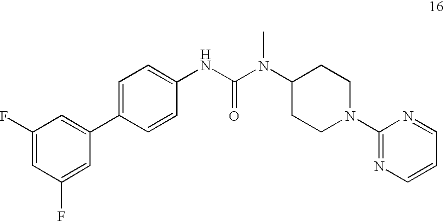

Reaction of the product of Example 15, Step 2 (15-2-1) and 4-1-1 with triphosgene by the procedure of Example 4, Step 4 gave the product. MS (m/e) 424 (M+H).

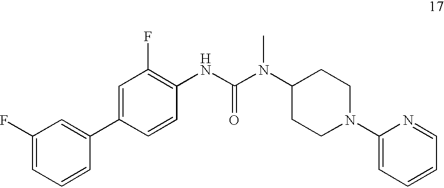

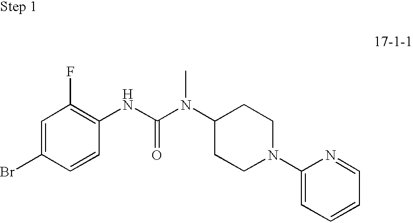

Reaction of the product of Example 5, Step 2 with 4-bromo-2-fluorophenylisocyanate by the procedure of Example 1, Step 1 gave the product. ¹H NMR (CDCl₃, 400 MHz) δ8.18 (1H, m), 7.47 (1H, m), 7.38 (2H, m), 7.30 (2H, m), 6.68 (1H, m), 6.61 (1H, m), 4.49 (1H, m), 4.43 (2H, m), 2.91 (2H, m), 2.85 (3H, s), 1.71 (4H,m). MS m/e 391 (M+H).

Step 2

Reaction of the product of Step 1 with 3-fluorophenylboronic acid by the procedure of Example 4, Step 1 gave the product. MS m/e 423 (M+H).

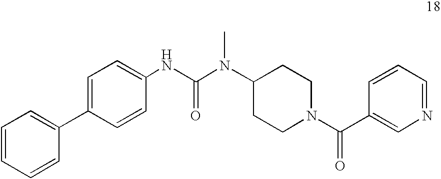

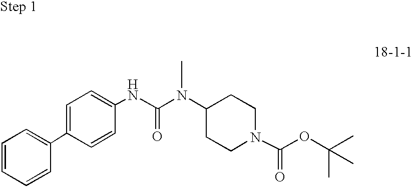

A mixture of 4-biphenyl isocyanate (3.00 g, 15.4 mmol) and Preparation 1 (5.33 g, 25.0 mmol) in CH₂Cl₂ (100 ml) was stirred at R.T. for 16 h. The mixture was washed with water (25 ml), 3N HCl (25 ml), and brine (50 ml). The organic portion was dried (MgSO₄), filtered, concentrated, and purified by column chromatography (gradient; CH₂Cl₂ to 1:99 CH₃OH/CH₂Cl₂) to give the product (6.11 g, 97%). MS (ES) m/e 410 (M+H)⁺.

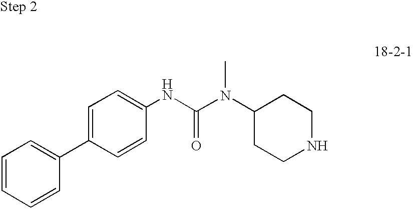

A mixture of the product of Step 1 (6.11 g, 14.9 mmol) and 4N HCl/dioxane (100 ml) was stirred at R.T. for 5 h. The volatiles were evaporated and the residue was triturated with ether. The precipitate was collected, dissolved in water (200 ml), basified to pH 14, and extracted with CH₂Cl₂ (300 ml). The organic portion was dried and concentrated to give the product (4.39 g, 92%). MS (ES) m/e 310 (M+H)⁺.

Step 3

A solution of the product of Step 2 (80 mg, 0.26 mmol), nicotinoyl chloride hydrochloride (54 mg, 0.30 mmol), and triethylamine (90 μl, 0.64 mmol) in CH₂Cl₂ (2 ml) was stirred at R.T. for 16 h. The mixture was diluted with CH₂Cl₂ (50 ml) and extracted with 3N NaOH (5 ml). The organic layer was washed with water (15 ml), dried, (MgSO₄), filtered, and concentrated. The residue was subjected to PTLC (4:96 CH₃OH/CH₂Cl₂) to give the product (90 mg, 84%). ¹H NMR (CDCl₃, 400 MHz) δ8.68 (2H, m), 7.76 (1H, m), 7.2-7.6 (1 OH, m), 6.48 (1H, s), 4.85 (1H, m), 4.60 (1H, m), 3.80 (1H, m), 3.20 (1H, m), 2.91 (3H, s), 2.86 (1H, m), 1.4-2.0 (4H, m). MS (ES) m/e 415 (M+H)⁺.

Using the appropriate acid chloride and essentially the same procedure the following compounds were prepared.

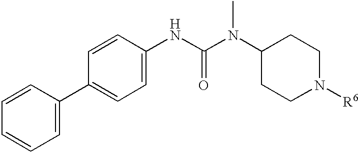

| R⁶ | (M + H)⁺ | Example |
|---|---|---|
| C(O)CH₃ | 352 | 18B |
| 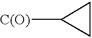 | 378 | 18C |
| 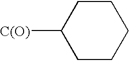 | 420 | 18D |
| 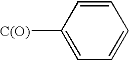 | 414 | 18E |
| 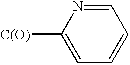 | 415 | 18F |
| 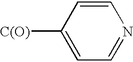 | 415 | 18G |

### EXAMPLE 19

Reaction of Example 1, 1-3-5, with the appropriate acid chloride afforded the following compounds:

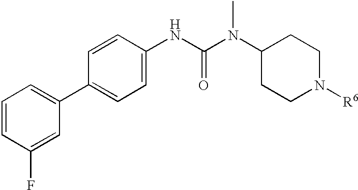

| R⁶ | (M + H)⁺ | Example |
|---|---|---|
| C(O)—CH₃ | 370 | 19A |
| 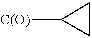 | 396 | 19B |
| 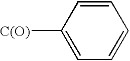 | 432 | 19C |
| 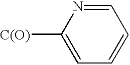 | 433 | 19D |
| 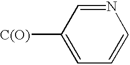 | 433 | 19E |
| 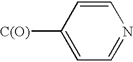 | 433 | 19F |
| 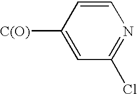 | 467 | 19G |
| 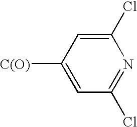 | 501 | 19H |
| 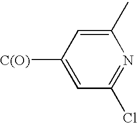 | 481 | 19I |
| 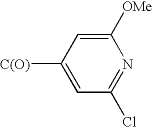 | 497 | 19J |

### EXAMPLE 20

Reaction of the product of Example 1, 1-3-7, with the appropriate acid chloride afforded the following compounds:

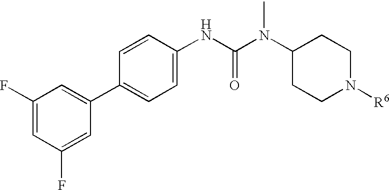

| R | (M + H)⁺ | Example |
|---|---|---|
| C(O)—CH₃ | 388 | 20A |
| 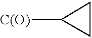 | 414 | 20B |
| 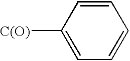 | 450 | 20C |
| 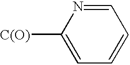 | 451 | 20D |
| 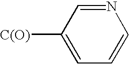 | 451 | 20E |
| 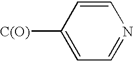 | 451 | 20F |

### EXAMPLE 21

Reaction of the product of Example 2, Step 5, 2-5-1, with the appropriate acid chloride afforded the following compounds

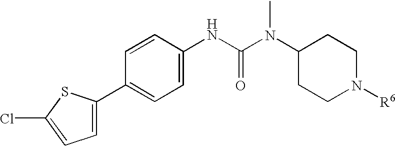

| R⁶ | (M + H)⁺ | Example |
|---|---|---|
| C(O)—CH₃ | 392 | 21A |
| 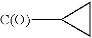 | 418 | 21B |
| 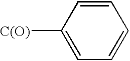 | 454 | 21C |
| 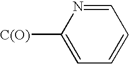 | 455 | 21D |
| 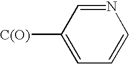 | 455 | 21E |
| 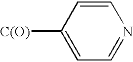 | 455 | 21F |

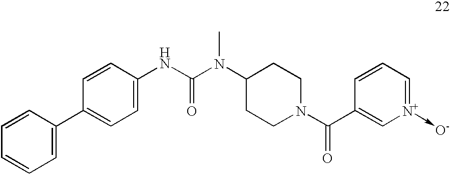

A mixture of Example 18 (45 mg, 0.11 mmol) and 3-chloroperoxybenzoic acid (40 mg) in CH₂Cl₂ (5 ml) was stirred at R.T. for 16 h. The mixture was diluted with CH₂Cl₂ (50 ml), then washed with 3N NaOH (2×5 ml) and water (10 ml). The organic layer was dried (Na₂SO₄), filtered, and concentrated. The residue was subjected to PTLC (1:9 CH₃OH/CH₂Cl₂) to give the product (34 mg, 73%). ¹H NMR (CDCl₃, 400 MHz) δ8.20 (2H, m), 7.2-7.6 (11H, m), 6.56 (1H, s), 4.76 (1H, m), 4.59 (1H, m), 3.78 (1H, m), 3.22 (1H, m), 2.7-3.0 (4H, m), 1.4-2.0 (4H, m). MS (ES) m/e 431 (M+H)⁺.

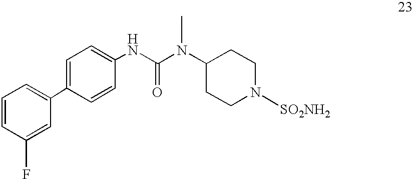

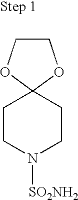

A mixture of 4-piperidone ethylene ketal (0.64 ml, 5.0 mmol) and sulfamide (0.53 g, 5.5 mmol) in DME (20 ml) was refluxed for 16 h. The mixture was concentrated to ca. 3 ml, dissolved in EtOAc (175 ml), washed with sat'd NH₄Cl (2×25 ml), water (2×25 ml), and brine (25 ml). The organic portion was dried, filtered, and evaporated to give the product (0.58 g, 52%). MS (ES) m/e 223 (M+H)⁺.

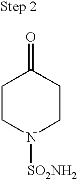

A mixture of the product of Step 1 (560 mg, 2.52 mmol) and pyridinium 4-toluenesulfonate (190 mg, 0.756 mmol) in acetone (25 ml) and water (0.5 ml) was refluxed for 64 h. The mixture was evaporated to dryness and the residue was partitioned between CH₂Cl₂ (75 ml) and aq. NaHCO₃ (2×20 ml). The aqueous layer was extracted with CH₂Cl₂ and EtOAc sequentially. The EtOAc layer was evaporated to give the product (140 mg). ¹H NMR (CD₃OD, 400 MHz) δ3.47 (1H, t, J=6.4 Hz), 3.15 (3H, m), 2.54 (1H, t, J=6.4 Hz), 1.81 (3H, m).

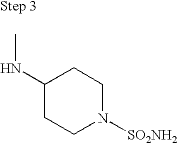

A mixture of the product of Step 2 (135 mg, 0.757 mmol), 40% aqueous methylamine (300 μl, 2.42 mmol), and sodium triacetoxyborohydride (375 mg, 1.77 mmol) in dichloroethane (5 ml) was stirred at R.T. for 19 h. The mixture was partitioned between 3N NaOH (5 ml) and EtOAc (3×50 ml). The organic layer was concentrated to give the crude product (40 mg). The aqueous layer was evaporated in vacuo to dryness and the residue was suspended in EtOAc. The suspension was filtered and the filtrate concentrated to give another batch of the product (70 mg). MS (FAB) m/e 194 (M+H)⁺.

Step 4

To an ice-cold solution of 4-1-2 (40 mg, 0.21 mmol) in anhydrous THF (3 ml) was added N,N′-disuccinimidyl carbonate (55 mg, 0.21 mmol) and pyridine (52 μl, 0.65 mmol). The mixture was stirred at 0° C. for 2 h and the product of Step 3 (70 mg, 0.36 mmol) was added. After stirring at R.T. for 2 h the reaction mixture was taken up in CH₂Cl₂ (50 ml), washed with 1N HCl (10 ml), dried, (Na₂SO₄), filtered and concentrated. The residue was subjected to PTLC (5:95 CH₃OH/CH₂Cl₂) to give the product (62 mg, 71%). ¹H NMR (CD₃OD, 400 MHz) δ7.56 (2H, m), 7.48 (2H, m), 7.40 (2H, m), 7.32 (1H, m), 7.02 (1H, m), 4.23 (1H, m), 3.75 (2H, m), 2.94 (3H, s), 2.72 (2H, m), 1.7-2.0 (4H, m). MS (ES) m/e 407 (M+H)⁺.

Using the appropriate starting materials and essentially the same procedure afforded the following compounds.

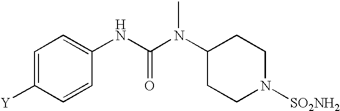

| Y | (M + H)⁺ | Example |
|---|---|---|
| 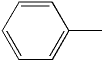 | 389 | 23A |
| 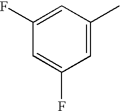 | 425 | 23B |
| 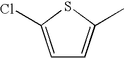 | 429 | 23C |

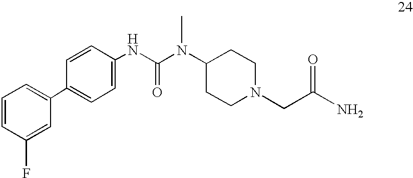

A mixture of 1-3-5 (71 mg, 0.20 mmol), 2-bromoacetamide (32 mg, 0.23 mmol), and anhydrous potassium carbonate (170 mg, 1.20 mmol) in CH₃CN (2 ml) in a sealed tube was heated to 45° C. for 6 h. The mixture was diluted with CH₂Cl₂ (75 ml), washed with water (50 ml), dried, and concentrated. The residue was subjected to PTLC (5:95 CH₃OH/CH₂Cl₂) to give the product (37 mg, 49%). ¹H NMR (CDCl₃, 400 MHz) δ7.48 (4H, m), 7.35 (2H, m), 7.23 (1H, m), 6.98 (2H, m), 6.56 (1H, s), 5.97 (1H, bs), 4.25 (1H, m), 2.8-3.0 (7H, m), 2.31 (2H, m), 1.6-1.8 (4H, m). MS (ES) m/e 385 (M+H)⁺.

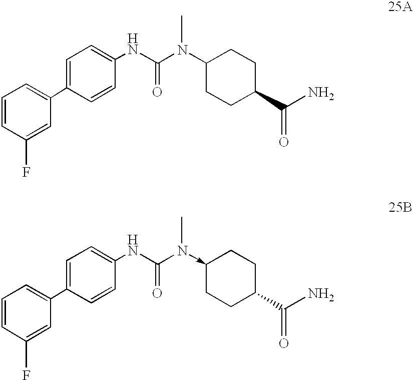

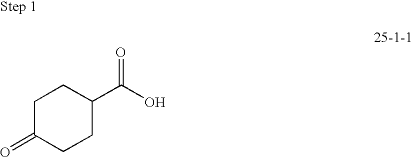

To ethyl 4-oxocyclohexanecarboxylate (10 g, 59 mmol) in MeOH (75 ml) and water (50 ml) was added lithium hydroxide monohydrate (4.2 g, 100 mmol) at 0° C. The mixture was warmed up to R.T. and stirred for 3 h. The mixture was acidified to pH 2 with 3N HCl. The volatiles were evaporated and the residue was extracted with EtOAc (300 ml). The organic portion was dried and concentrated to give the product (8.01 g, 96%). MS (Cl) m/e 143 (M+H)⁺.

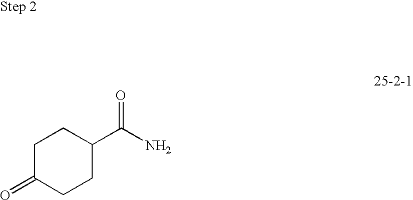

2M oxalyl chloride in CH₂Cl₂ (20 ml, 40 mmol) was added over 5 min to a solution of the product of Step 1 (3.0 g, 21 mmol) in anhydrous THF (50 ml). The solution was heated to 80° C. for 6 h and then evaporated to dryness. The residue was dissolved in THF (50 ml) at 0° C. and aq. NH₄OH (6.0 ml, 89 mmol) was added. After stirring at R.T. for 16 h, the mixture was concentrated and the residue purified by column chromatography (gradient CH₂Cl₂ to 2:98 CH₃OH/CH₂Cl₂) to give the product (762 mg, 26%). MS (Cl) m/e 142 (M+H)+.

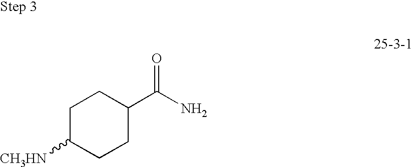

A mixture of the product of Step 2 (800 mg, 5.71 mmol), 40% aq. methylamine (4.0 ml, 52 mmol), and sodium triacetoxyborohydride (1.7 g, 8.0 mmol) in dichloroethane (20 ml) was stirred at R.T. for 16 h. The reaction was quenched with 3N NaOH and partitioned between brine and 1:1 CH₃CN/CH₂Cl₂. The organic portion was concentrated and the residue purified by column chromatography (gradient CH₂Cl₂ to 1:4 2M NH₃ in CH₃OH/CH₂Cl₂) to give the product (450 mg, 51%). MS (Cl) m/e 157 (M+H)⁺.

Step 4

A mixture of the aniline 4-1-2 (100 mg, 0.534 mmol), N,N′-disuccinimidyl carbonate (137 mg, 0.535 mmol), and pyridine (0.13 ml, 1.6 mmol) in THF (3 ml) was stirred at 0° C. for 2 h. To this mixture was added the product of Step 3 (125 mg, 0.811 mmol) and the reaction was stirred at R.T. for 2 h. The mixture was diluted with CH₂Cl₂ (100 ml), washed with 1N HCl (2×25 ml), water (2×25 ml), brine (25 ml), dried, and concentrated. The residue was subjected to PTLC (3:97 CH₃OH/CH₂Cl₂) to give the cis-product (14 mg) and the trans-product (15 mg).

cis-product 25A: ¹H NMR (CD₃OD, 400 MHz): δ7.4-7.6 (4H, m), 7.33 (2H, m), 7.22 (1H, m), 6.95 (1H, m), 4.13 (1H, m), 2.86 (3H, s), 2.53 (1H, m), 2.13 (2H, m), 1.82 (2H, m), 1.5-1.75 (4H, m). MS (ES) m/e 370 (M+H)⁺.

trans-product 25B: ¹H NMR (CD₃OD, 400 MHz): δ7.4-7.5 (4H, m), 7.34 (2H, m), 7.23 (1H, m), 6.96 (1H, m), 4.07 (1H, m), 2.88 (3H, s), 2.14 (1H, m), 1.98 (2H, m), 1.81 (2H, m), 1.5-1.7 (4H, m). MS (ES) m/e 370 (M+H)⁺.

Reaction of the product of Step 3, 25-3-1 with aniline 4-1-1 by essentially the same procedure gave 25C and 25D:

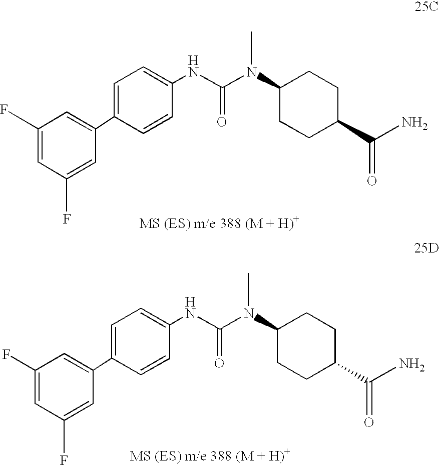

### EXAMPLE 26

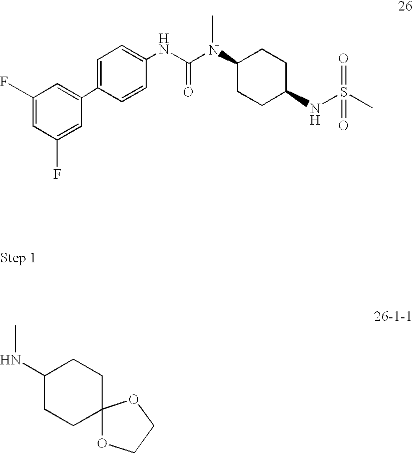

To a stirred mixture of 1,4-cyclohexanedione monoethylene ketal (4.68 g, 30 mmol) and 40% w/w aq. methylamine (6.0 mL) in 1,2-dichloroethane (75 mL), was added Na(OAc)₃BH (9.6 g, 45 mmol) in portions. The reaction mixture was vigorously stirred for 16 h, then 1N NaOH (75 mL) was added. The organic layer was washed with sat'd NaCl, dried (MgSO₄), filtered, and evaporated to give an oil (4.60 g, 90%) that was used without further purification. ¹H NMR (CDCl₃, 400 MHz) δ3.97 (4H, s), 2.47 (1H, m), 2.46 (3H, s), 1.91 (2H, m), 1.80 (2H, m), 1.59 (2H, m), 1.45 (2H, m).

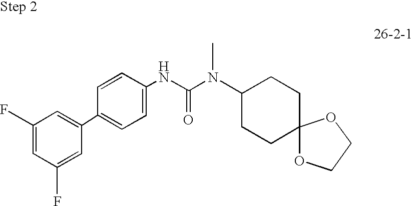

To a stirred, ice-cold mixture of aniline 4-1-1 (1.00 g, 4.87 mmol) and pyridine (1.97 ml, 24.3 mmol) in anhydrous THF (50 ml) was added disuccinimidyl carbonate (1.25 g, 4.87 mmol). The reaction mixture was stirred at 0° C. for 1 h and the product of Step 1 (1.25 g, 7.31 mmol) was added. The reaction mixture was allowed to warm to R.T., stirred for 16 h, then poured into cold H₂O (100 ml). The whole was extracted with CH₂Cl₂ (3×100 ml). The combined organic layers were dried (Na₂SO₄), filtered, and evaporated. Purification of the residue by column chromatography (1:20 CH₃OH/CH₂Cl₂) afforded the product (1.40 g, 71%). ¹H NMR (CDCl₃, 400 MHz) δ7.49 (4H, m), 7.10 (2H, m), 6.70 (1H, m), 6.60 (1H, s), 4.30 (1H, m), 3.90 (4H, s), 2.90 (3H, s), 1.75 (8H, m). MS m/e 403 (M+H).

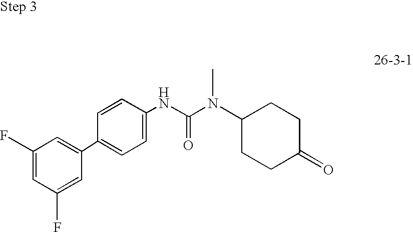

To the product of Step 2 (1.30 g, 3.23 mmol) in THF (30 ml) was added 5N HCl (20 ml). The reaction mixture was stirred at R.T. for 4.5 h, then extracted with CH₂Cl₂ (3×100 ml). The combined organic extracts were washed with sat'd NaHCO₃, dried (Na₂SO₄), filtered and evaporated. The residue was purified by PTLC (1:20 CH₃OH/CH₂Cl₂) to give the product (0.80 g, 69%). ¹H NMR (CDCl₃, 400 MHz) δ7.50 (4H, m), 7.10 (2H, m), 6.80 (1H, m), 6.50 (1H, s), 4.80 (1H, m), 2.90 (3H, s), 2.48 (4H, m), 2.10 (2H, m), 1.90 (2H, m). MS m/e 359 (M+H).

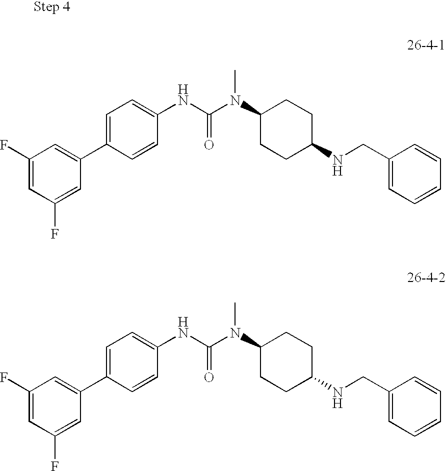

To a mixture of the product of Step 3 (0.43 g, 1.20 mmol) and benzylamine (0.257 g, 2.40 mmol) in 1,2-dichloroethane (10 ml) was added NaBH(OAc)₃ (0.762 g, 3.60 mmol) in portions. The reaction mixture was stirred at R.T. for 4.5 h, then poured into sat'd NaHCO₃ (20 ml) and extracted with CH₂Cl₂ (3×20 ml). The combined organic layers were dried (Na₂SO₄), filtered and evaporated. The residue was purified by PTLC (1:20 (2M NH₃/CH₃OH):CH₂Cl₂) to produce the cis-isomer 26-4-1 (0.240 g, 44.5%) and the trans-isomer 26-4-2 (0.200 g, 37.0%). Cis isomer: ¹H NMR (CDCl₃, 400 MHz) δ7.48 (4H, m), 7.30 (5H, m), 7.05 (2H, m), 6.70 (1H, m), 6.40 (1H, s), 4.20 (1H, m), 3.78 (2H, s), 2.90 (4H, m), 1.90 (4H, m), 1.55 (4H, m). MS m/e 450 (M+H). Trans-isomer: ¹H NMR (CDCl₃, 400 MHz) δ7.48 (4H, m), 7.33 (5H, m), 7.05 (2H, m), 6.70 (1H, m), 6.37 (1H s), 4.20 (1H, m), 3.82 (2H, s), 2.88 (3H, m), 2.50 (1H, m), 2.10 (2H, m), 1.80 (2H, m), 1.20-1.70 (4H, m). MS m/e 450 (M+H).

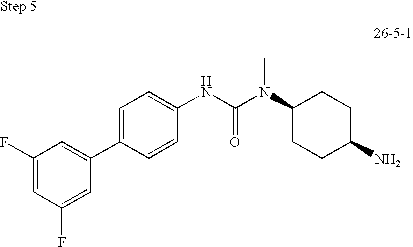

To the cis isomer 26-4-1 (0.600 g, 1.33 mmol) in 4.4% HCOOH/CH₃OH (50 ml) was added 10% Pd/C (0.600 g). The reaction mixture was stirred at R.T. under argon for 16 h, then filtered through celite and concentrated. The residue was purified by PTLC (1:10 (2M NH₃/CH₃OH)/CH₂Cl₂) to afford the product (0.230 g, 85%). ¹H NMR (CDCl₃, 400 MHz) δ7.50 (4H, s), 7.06 (2H, m), 6.70 (1H, m), 6.40 (1H, s), 4.20 (1H, m), 3.30(1H), 3.00 (3H, s), 1.50-2.30 (10H, m). MS m/e 360 (M+H).

Step 6

To a mixture of the product of Step 5 (0.140 g, 0.390 mmol) and 1M K₂CO₃ (1.2 ml, 1.2 mmol) in THF (5 ml) was added MeSO₂Cl (0.178 g, 1.55 mmol). The reaction mixture was stirred at R.T. for 16 h then subjected to PTLC (1:10 CH₃OH/CH₂Cl₂) to give the product (0.135 g, 79%). ¹H NMR (CDCl₃, 400 MHz) δ7.53 (4H, m), 7.20 (2H, m), 6.90 (1H, m), 4.10 (1H, m), 3.60 (1H, m), 2.90 (6H, s) 1.50-2.10 (8H, m). MS m/e 438 (M+H).

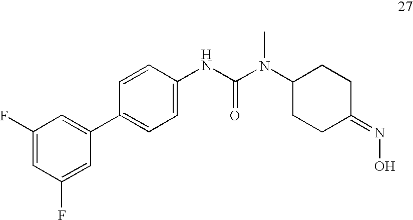

A mixture of 26-3-1 (0.21 g, 0.59 mmol), hydroxylamine hydrochloride (0.82 g, 12 mmol), and sodium acetate (0.97 g, 12 mmol) in absolute EtOH (10 ml) was stirred at R.T. for 64 h. The mixture was partitioned between CH₂Cl₂ (100 ml) and water (75 ml). The aqueous layer was extracted again with CH₂Cl₂ (50 ml). The combined organic layers were dried (Na₂SO₄), filtered and concentrated. The residue was subjected to PTLC (1:19 CH₃OH/CH₂Cl₂) to give the product (210 mg, 95%). ¹H NMR (CD₃OD, 400 MHz) δ7.4-7.6 (4H, m), 7.20 (2H, m), 6.85 (1H, m), 4.39 (1H, m), 3.45 (1H, m), 2.90 (3H, s), 2.45 (1H, m), 2.28 (1H, m), 1.6-2.0 (5H, m). MS (ES) m/e 374 (M+H).

Use of the appropriate starting material and essentially the same procedure afforded the following compound.

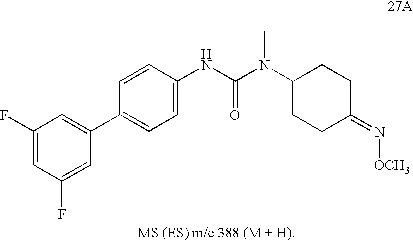

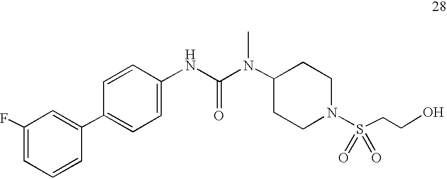

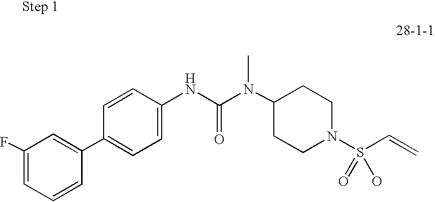

To a mixture of 1-3-5 (100 mg, 0.31 mmol), 1 M NaOH (0.5 ml), and 1 M Na₂CO₃ (0.5 ml) in CH₂Cl₂ (5 ml) was added 2-chloroethylsulfonyl chloride (100 mg, 0.61 mmol), and the reaction mixture was stirred for 16 hr. The reaction mixture was partitioned between water (25 ml) and CH₂Cl₂ (25 ml). The organic layer was dried (MgSO₄), filtered, and concentrated. Subjection of the residue to PTLC (1:4 acetone/CH₂Cl₂) gave the product (40 mg, 31%). MS (ES) m/e 418 (M+H).

Step 2

To a stirred solution of the product of Step 1 (28-1-1) (50 mg, 0.12 mmol) in THF (10 ml) was added tetrabutylammonium hydroxide (0.5 g) in water (2 ml). After 16 hr, the reaction mixture was partitioned between water (25 ml) and CH₂Cl₂ (100 ml). The organic layer was dried (MgSO₄), filtered, and concentrated. Subjection of the residue to PTLC (5:95 MeOH/CH₂Cl₂) gave the product (24 mg, 46%). HRMS calc. for C₂₁H₂₇FN₃O₄S (M+H) 436.1706. Found 436.1711.

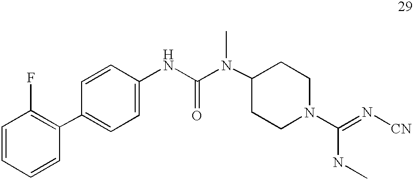

To a solution of 1-3-1 (400 mg, 1.22 mmol) in DMF (5 ml) was added EDCI (25 mg, 1.30 mmol) and 1-cyano-3-methylisothiourea sodium salt (175 mg, 1.27 mmol). The reaction mixture was stirred for 16 h, then diluted with EtOAc (50 ml). The mixture was washed with water (10 ml), sat'd NaHCO₃ (20 ml) and water (10 ml). The organic layer was dried (MgSO₄), filtered and concentrated. Subjection of the residue to flash chromatography (gradient; 3:97-7:93 MeOH/CH₂Cl₂) gave the product (250 mg, 50%). HRMS calc. For C₂₂H₂₆N₆OF (M+H) 409.2152. Found 409.2155.

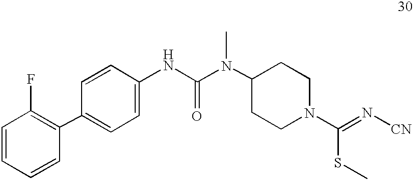

To a solution of 1-3-1 (500 mg, 1.53 mmol) in acetonitrile (10 ml) was added dimethyl-N-cyanodithioiminocarbonate (0.8 g, 5.5 mmol) and the reaction mixture was refluxed for 16 h. The reaction mixture was poured into water (50 ml) and extracted with EtOAc (50 ml). The organic layer was dried (MgSO₄), filtered and concentrated. Subjection of the residue to flash chromatography (1:2 acetone/hexanes) gave the product (150 mg, 24%). MS m/e 426.1 (M+H).

Method for Screening Compound 14 of Example 14 for Y5 Antagonist Activity In Vivo

Adult male Long-Evans or Sprague-Dawley rats (200-250 g, Charles River, Ma.) were maintained in individual cages at 22° C. on a 12 hr light/12 hr dark cycle with lights on at 0400. Rats had free access to food (Teklad Lab Rodent Chow, Bartonville, Ill.) and water. All studies were conducted in an AAALAC accredited facility following protocols approved by the Animal Care and Use Committee of the Schering-Plough Research Institute. The procedures were performed in accordance with the principles and guidelines established by the NIH for the care and use of laboratory animals.

Rats were anesthetized by intramuscular injection of a mixture of ketamine and xylazine (100 and 10 mg/kg, respectively). A 22 gauge stainless steel cannula was stereotaxically implanted into the lateral ventricle using the following coordinates: 1 mm posterior to bregma, 1.5 mm lateral to midline, 3.6 mm ventral to dura. After a three week recovery period, all animals were tested for correct cannula placement by intracerebroventricular (icv) infusion of human NPY (0.3 nmol). Only animals demonstrating a profound feeding effect (>2 g) within 60 min of the infusion were retained for the study. Four groups of twelve animals were used in each study. Each group was balanced such that the average baseline and NPY-induced food intake values were similar for each group. One group received an oral dose of vehicle while the other three groups received oral doses of the Y5 antagonist 14 one hour before icv administration of D-Trp34-NPY. D-Trp34-NPY was dissolved in 0.9% sterile saline (Sigma, St. Louis, Mo.) and were infused icv with a Hamilton infusion pump and syringe (Hamilton, Reno, Nev.) at a rate of 5 μl/min. The guide cannula remained inserted for an additional minute to prevent diffusion up the needle track. The chow-filled feeder was weighed during the infusion period and then returned to the home cage with the animal immediately following treatment. Food consumption was monitored at 60, 120 and 240 min after icv infusion of peptides. Differences in food intake between groups were determined by analysis of variance followed by Dunnett's multiple comparison test. Compound 14 (0.1, 0.3, 1, and 3 mg/kg) dose responsively inhibited D-Trp34-NPY stimulated food intake with an ID50 of 0.5 mg/kg.

It will be recognized that the following examples can be prepared by adapting appropriate procedures described in Examples 1-30, or by applying methods known to those skilled in the art:

| Example | Structure | MSm/e (M + H) |
|---|---|---|
| 31 | 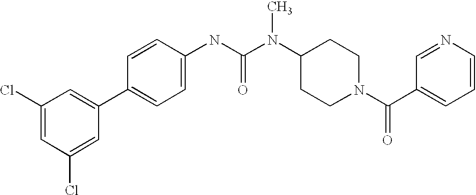 | 483 |
| 32 | 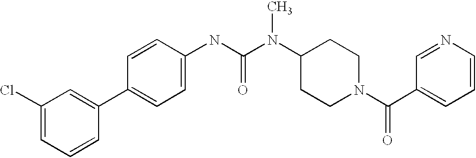 | 449 |
| 33 | 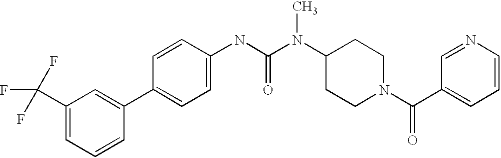 | 483 |
| 34 | 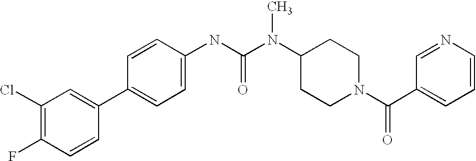 | 467 |
| 35 | 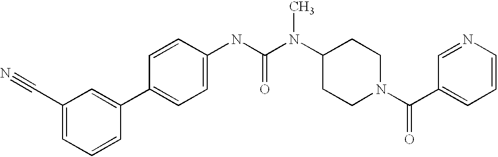 | 440 |
| 36 | 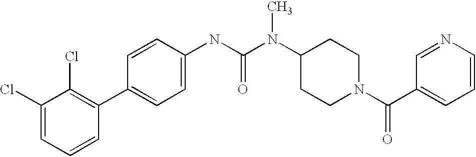 | 483 |
| 37 | 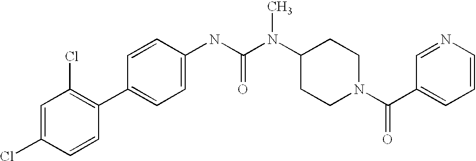 | 483 |
| 38 | 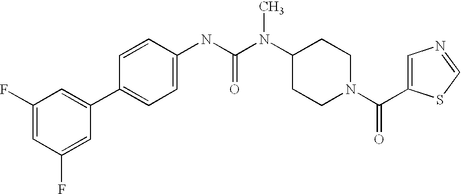 | 457 |
| 39 | 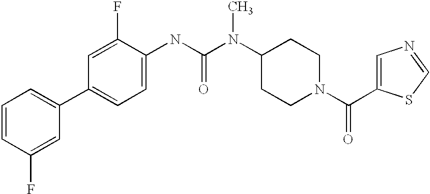 | 457 |
| 40 | 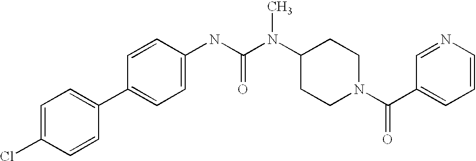 | 449 |
| 41 | 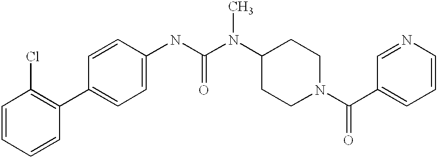 | 449 |
| 42 | 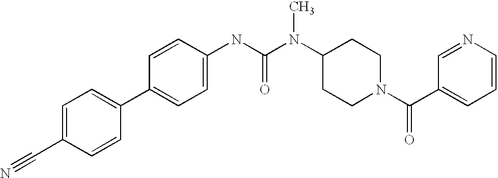 | 440 |
| 43 | 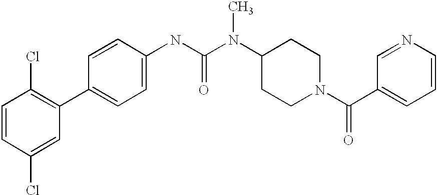 | 483 |
| 44 | 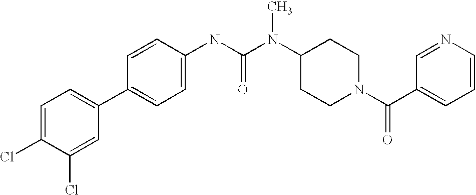 | 483 |
| 45 | 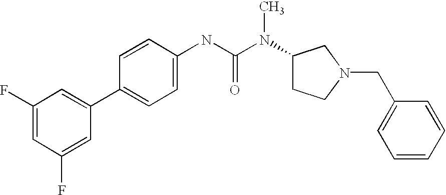 | 422 |
| 46 | 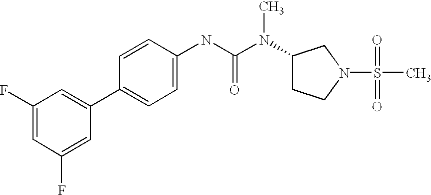 | 410 |
| 47 | 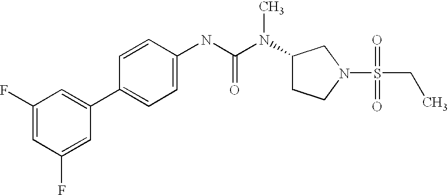 | 424 |
| 48 | 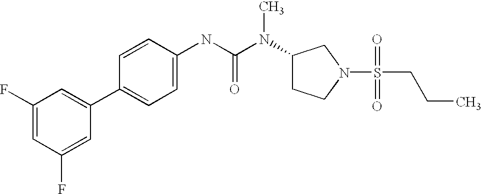 | 438 |
| 49 | 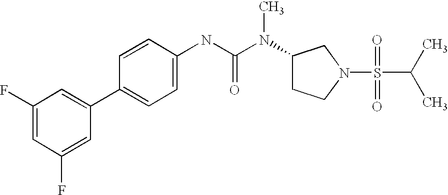 | 438 |
| 50 | 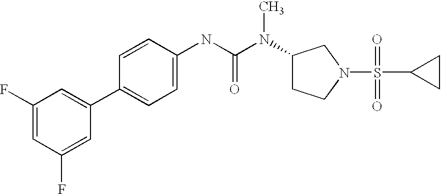 | 436 |
| 51 | 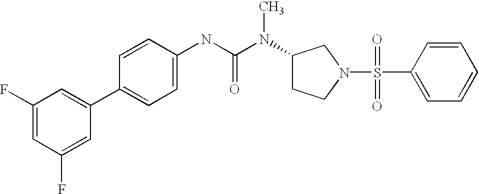 | 472 |
| 52 | 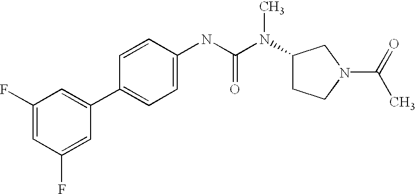 | 374 |
| 53 | 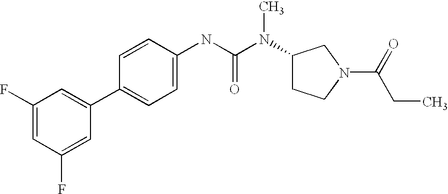 | 388 |
| 54 | 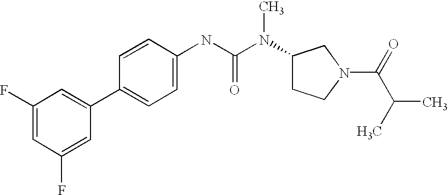 | 402 |
| 55 | 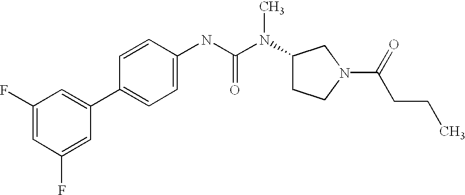 | 402 |
| 56 | 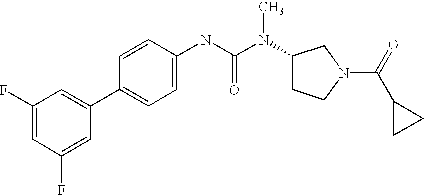 | 400 |
| 57 | 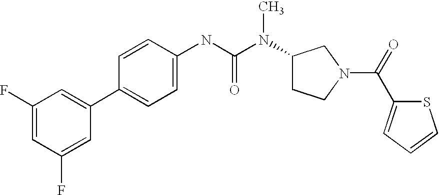 | 442 |
| 58 | 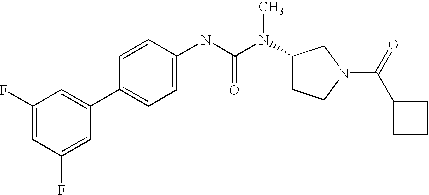 | 414 |
| 59 | 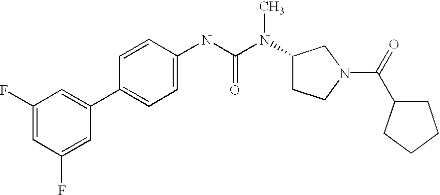 | 428 |
| 60 | 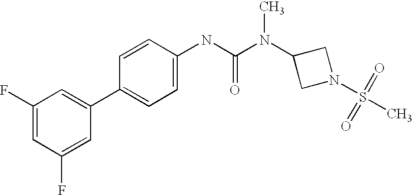 | 396 |
| 61 | 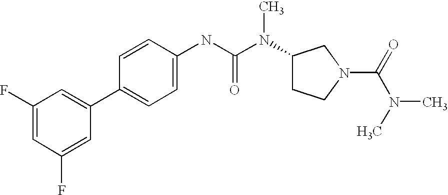 | 403 |
| 62 | 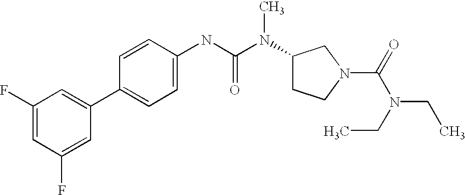 | 431 |
| 63 | 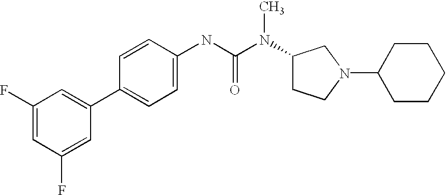 | 414 |
| 64 | 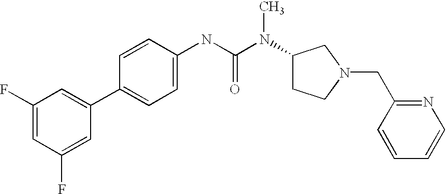 | 423 |
| 65 | 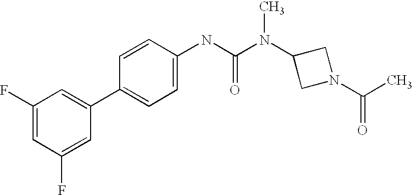 | 360 |
| 66 | 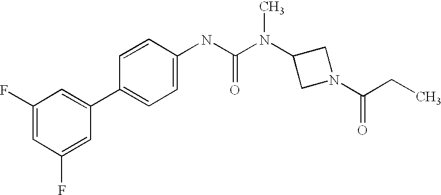 | 374 |
| 67 | 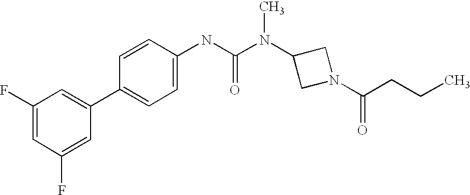 | 388 |
| 68 | 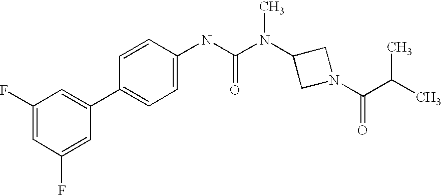 | 388 |
| 69 | 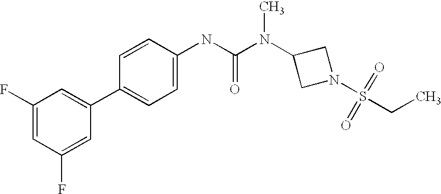 | 410 |
| 70 | 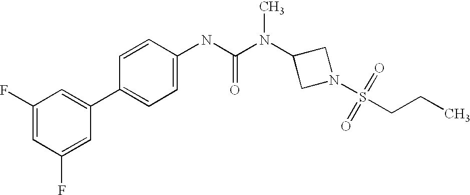 | 424 |
| 71 | 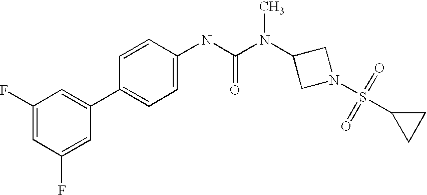 | 422 |
| 72 | 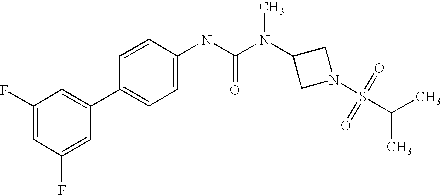 | 424 |
| 73 | 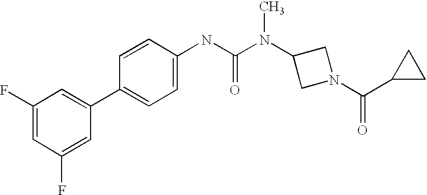 | 386 |
| 74 | 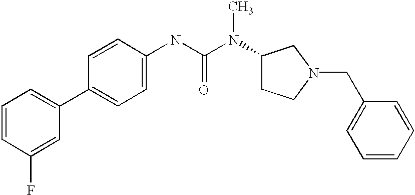 | 404 |
| 75 | 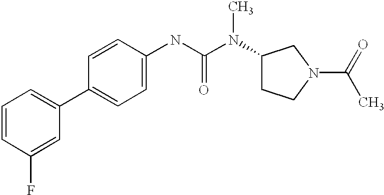 | 356 |
| 76 | 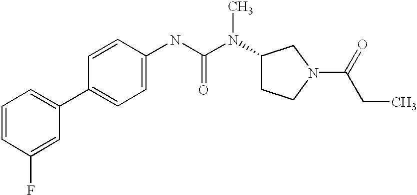 | 370 |
| 77 | 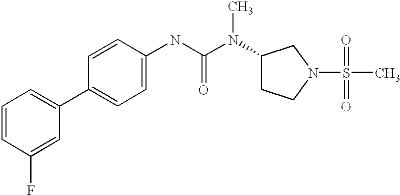 | 392 |
| 78 | 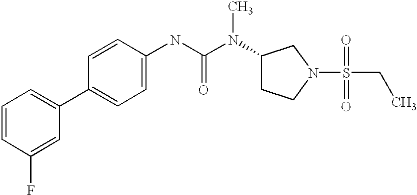 | 406 |
| 79 | 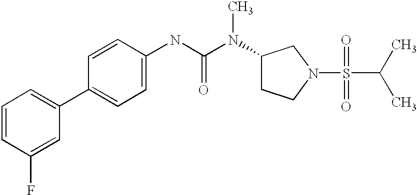 | 420 |
| 80 | 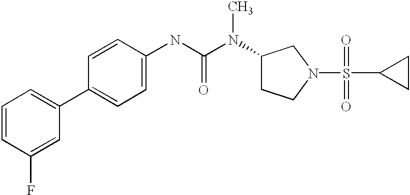 | 418 |
| 81 | 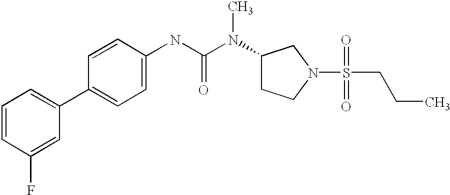 | 420 |
| 82 | 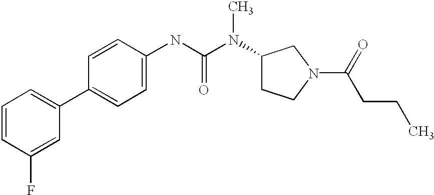 | 384 |
| 83 | 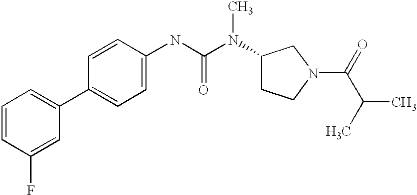 | 384 |
| 84 | 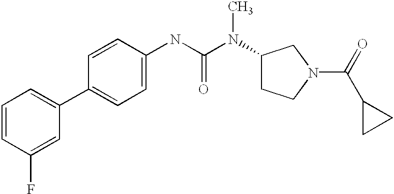 | 382 |
| 85 | 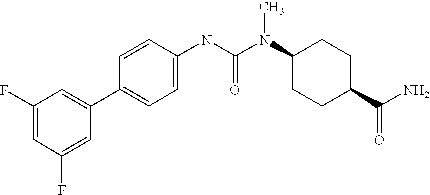 | 388 |
| 86 | 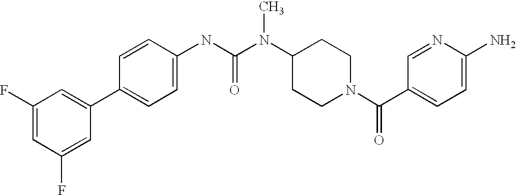 | 466 |
| 87 | 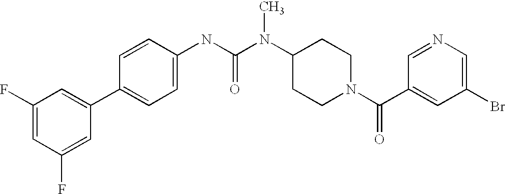 | 531 |
| 88 | 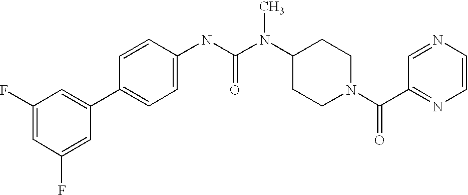 | 452 |
| 89 | 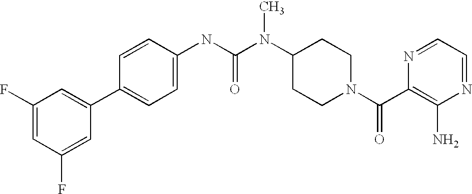 | 467 |
| 90 | 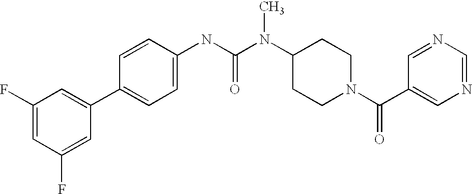 | 452 |
| 91 | 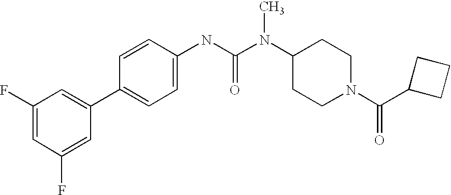 | 428 |
| 92 | 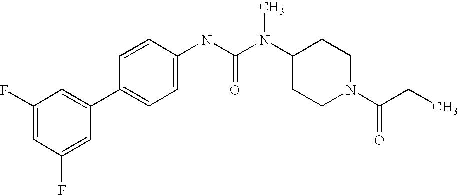 | 402 |
| 93 | 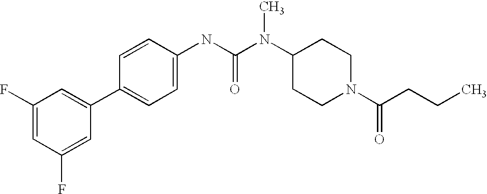 | 416 |
| 94 | 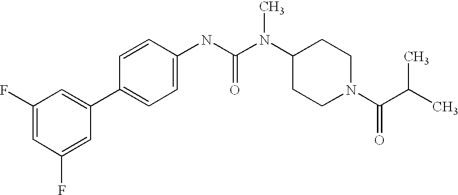 | 416 |
| 95 | 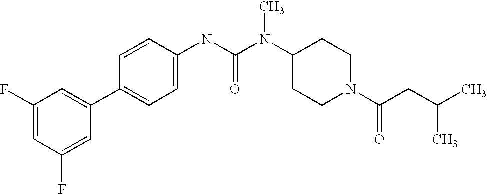 | 430 |
| 96 | 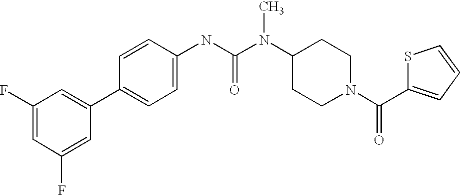 | 456 |
| 97 | 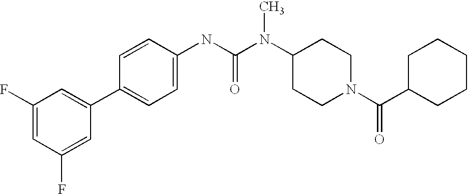 | 456 |
| 98 | 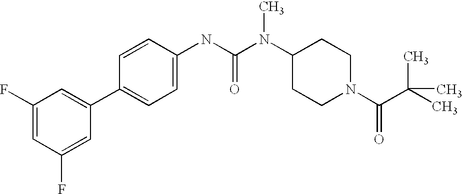 | 430 |
| 99 | 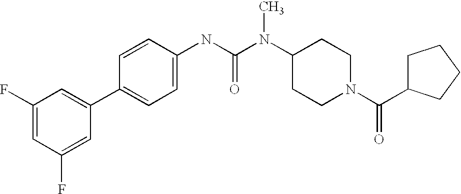 | 442 |
| 100 | 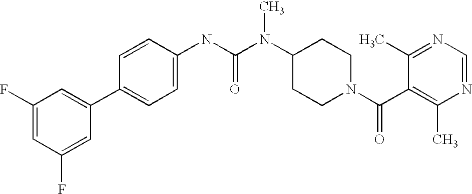 | 480 |
| 101 | 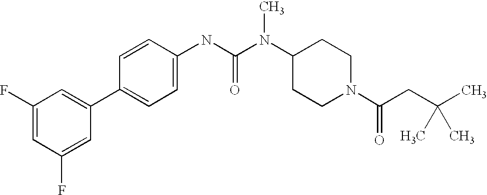 | 444 |
| 102 | 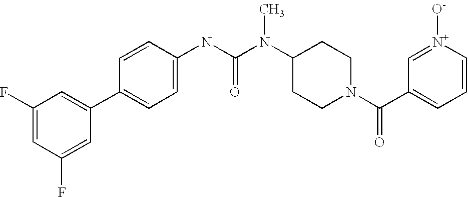 | 467 |
| 103 | 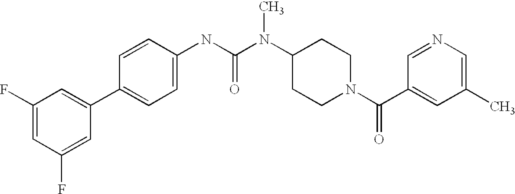 | 465 |
| 104 | 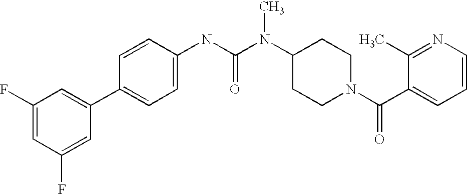 | 465 |
| 105 | 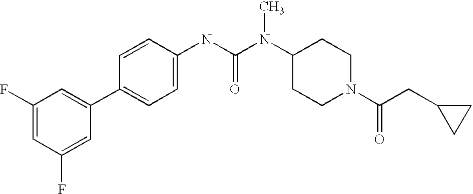 | 428 |
| 106 | 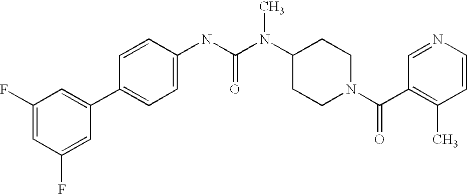 | 465 |
| 107 | 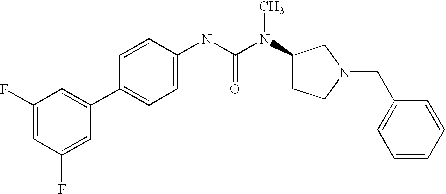 | 422 |
| 108 | 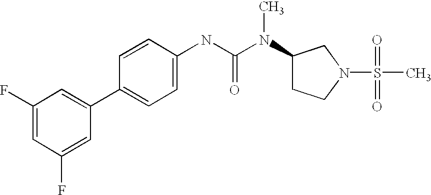 | 410 |
| 109 | 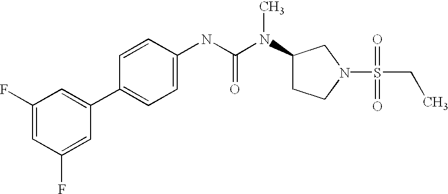 | 424 |
| 110 | 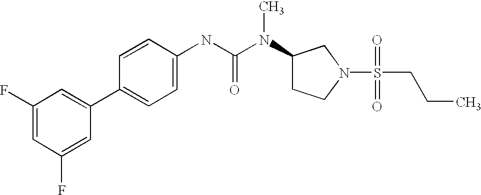 | 438 |
| 111 | 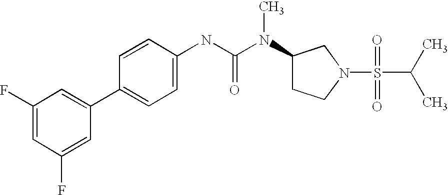 | 438 |
| 112 | 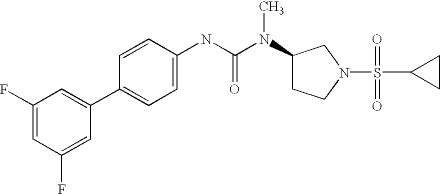 | 436 |
| 113 | 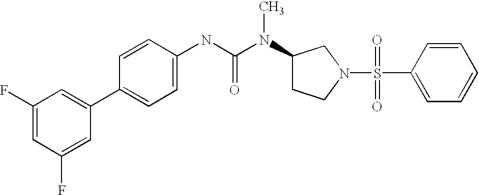 | 472 |
| 114 | 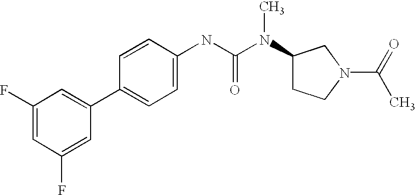 | 374 |
| 115 | 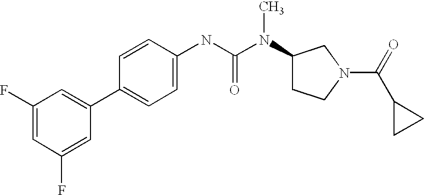 | 400 |
| 116 | 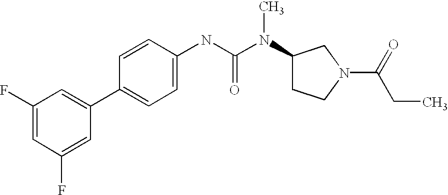 | 388 |
| 117 | 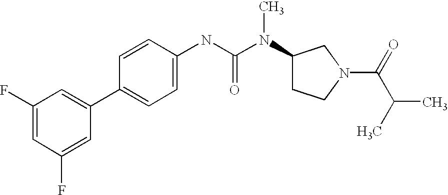 | 402 |
| 118 | 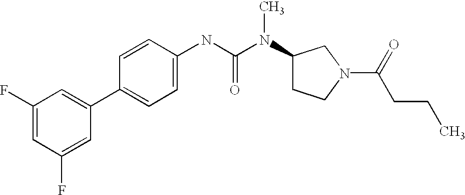 | 402 |
| 119 | 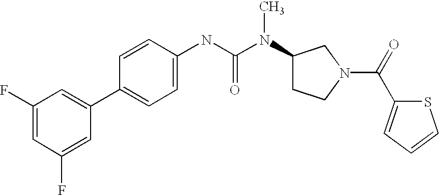 | 442 |
| 120 | 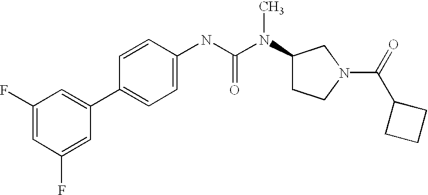 | 414 |
| 121 | 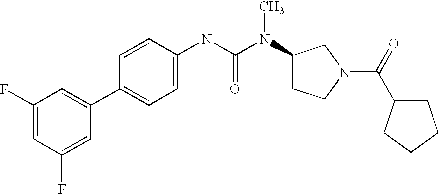 | 428 |
| 122 | 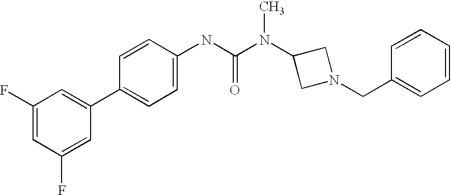 | 408 |
| 123 | 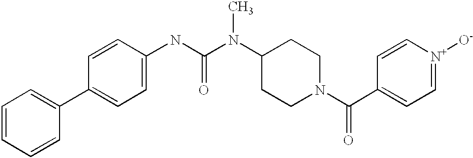 | 431 |
| 124 | 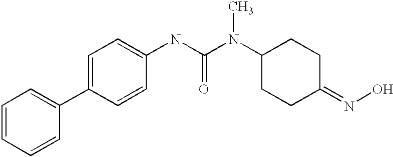 | 338 |
| 125 | 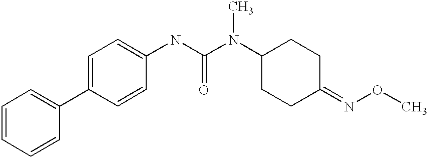 | 352 |
| 126 | 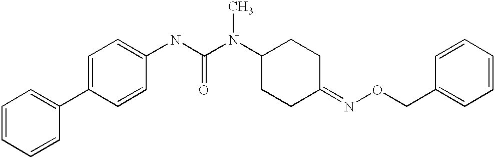 | 428 |
| 127 | 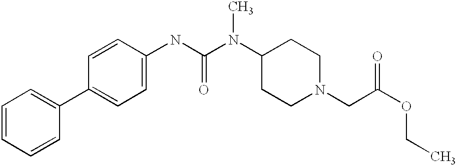 | 396 |
| 128 | 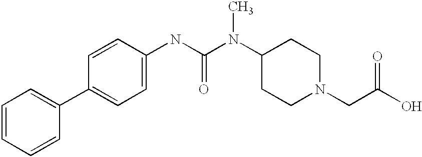 | 368 |
| 129 | 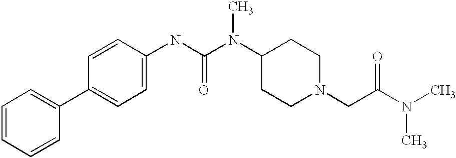 | 395 |
| 130 | 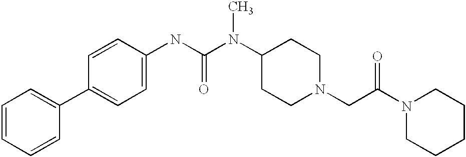 | 435 |
| 131 | 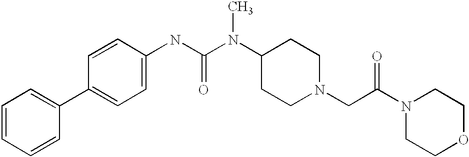 | 437 |
| 132 | 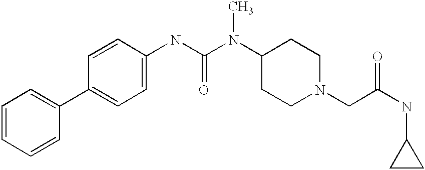 | 407 |
| 133 | 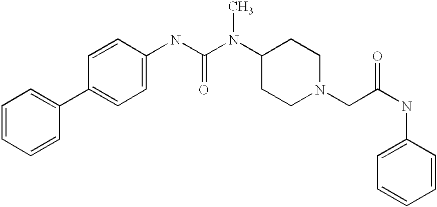 | 443 |
| 134 | 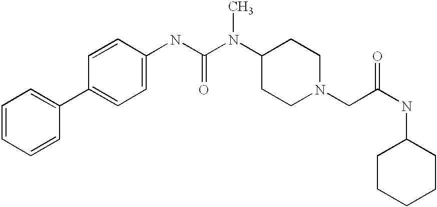 | 449 |
| 135 | 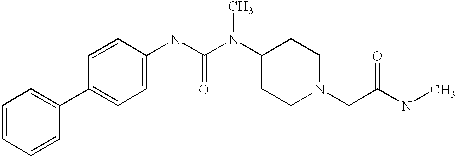 | 381 |
| 136 | 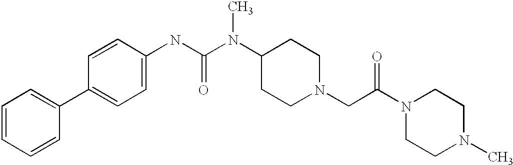 | 450 |
| 137 | 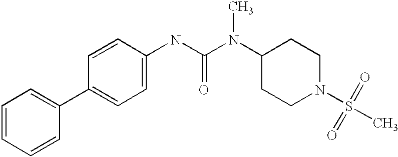 | 388 |
| 138 | 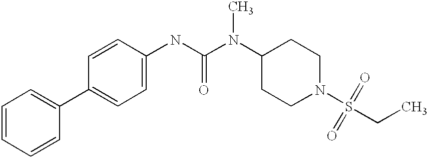 | 402 |
| 139 | 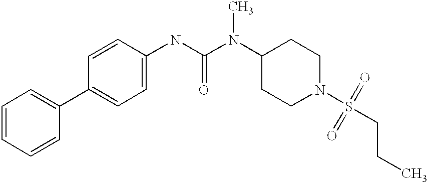 | 416 |
| 140 | 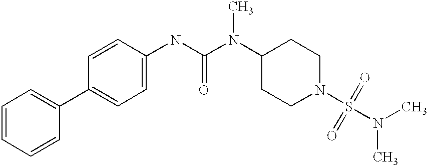 | 417 |
| 141 | 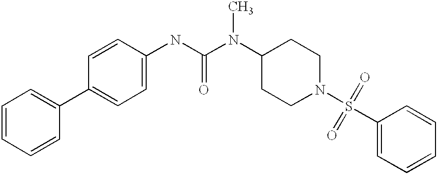 | 450 |
| 142 | 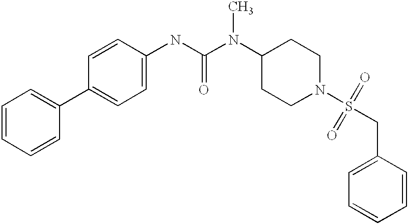 | 464 |
| 143 | 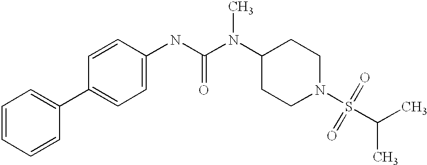 | 416 |
| 144 | 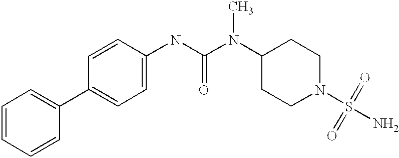 | 389 |
| 145 | 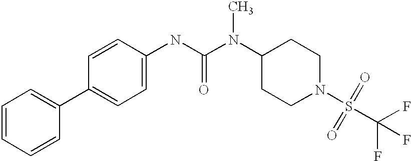 | 442 |
| 146 | 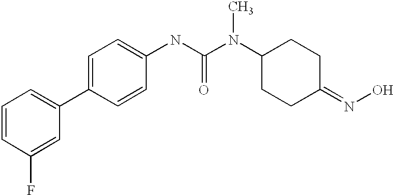 | 356 |
| 147 | 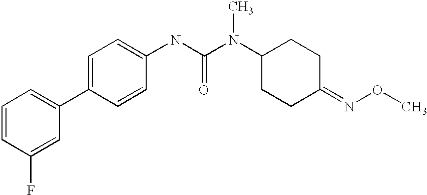 | 370 |
| 148 | 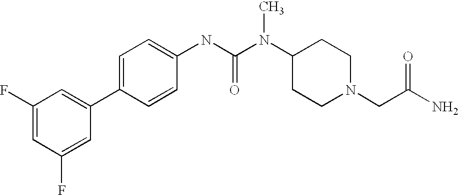 | 403 |
| 149 | 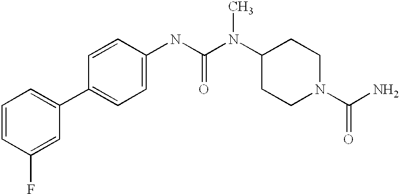 | 371 |
| 150 | 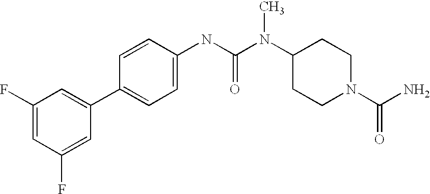 | 389 |
| 151 | 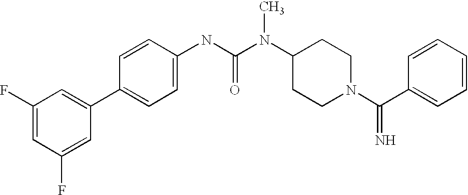 | 449 |
| 152 | 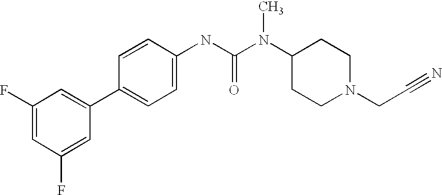 | 385 |
| 153 | 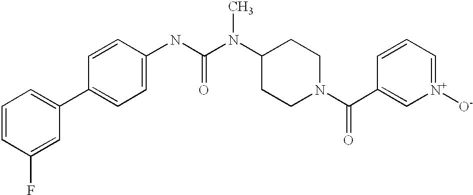 | 449 |
| 154 | 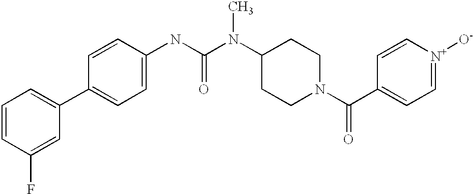 | 449 |
| 155 | 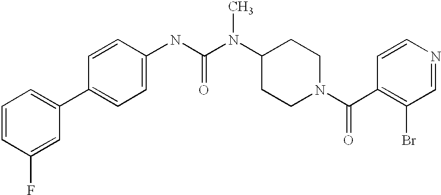 | 511 |
| 156 | 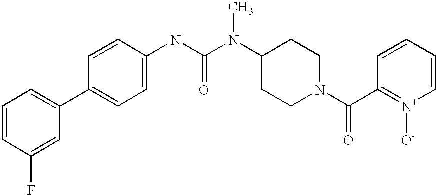 | 449 |
| 157 | 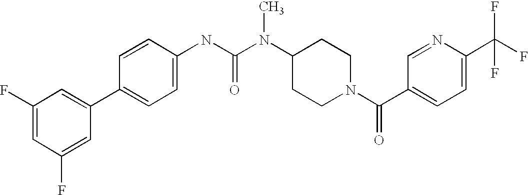 | 519 |
| 158 | 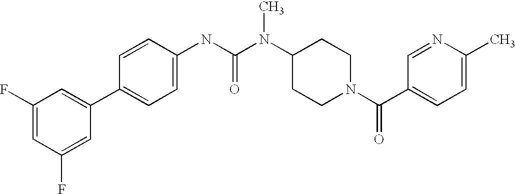 | 465 |
| 159 | 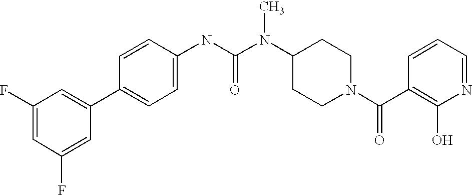 | 467 |
| 160 | 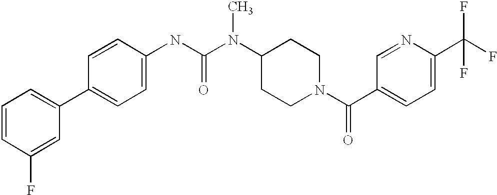 | 501 |
| 161 | 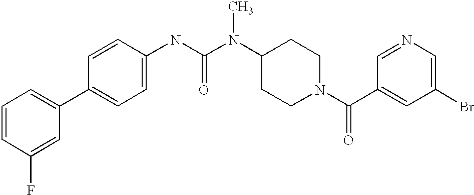 | 511 |
| 162 | 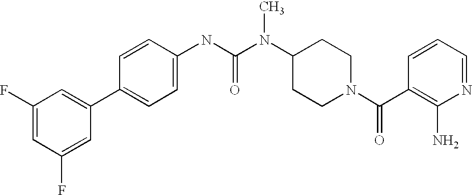 | 466 |
| 163 | 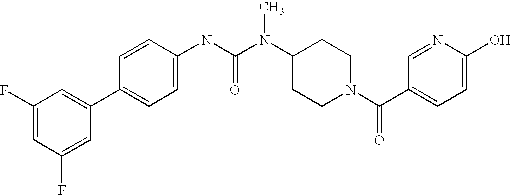 | 467 |
| 164 | 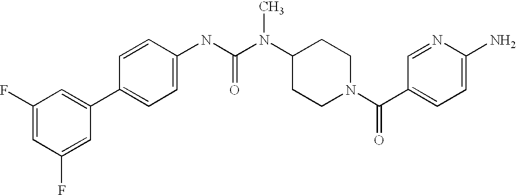 | 466 |
| 165 | 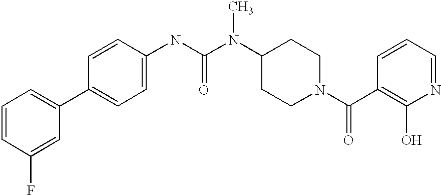 | 449 |
| 166 | 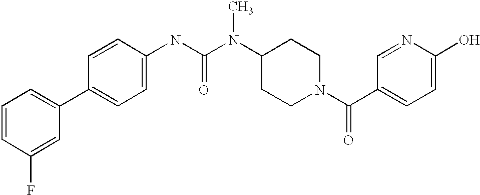 | 449 |
| 167 | 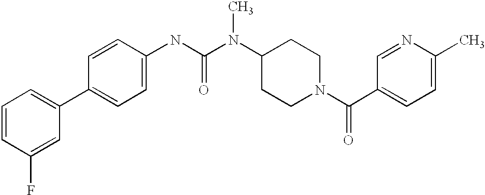 | 447 |
| 168 | 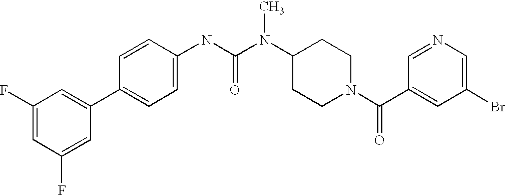 | 531 |
| 169 | 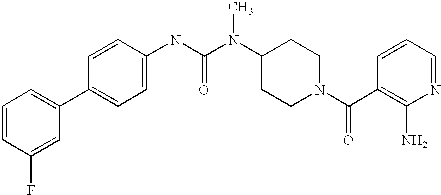 | 448 |
| 170 | 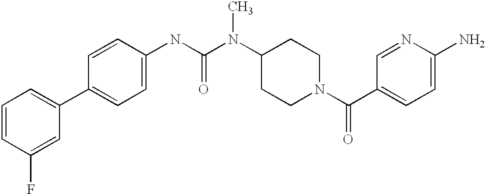 | 448 |
| 171 | 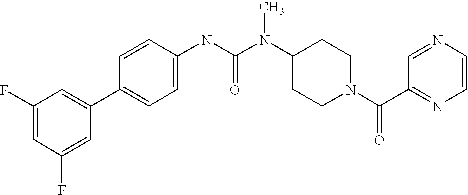 | 452 |
| 172 | 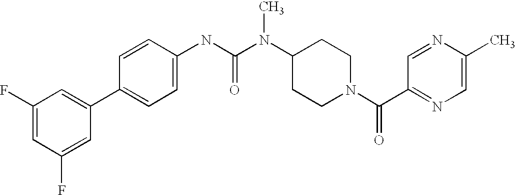 | 466 |
| 173 | 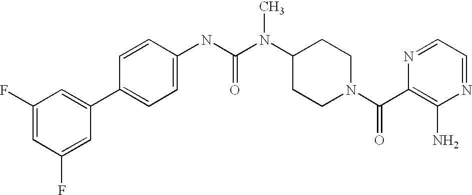 | 467 |
| 174 | 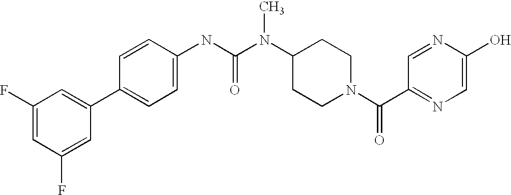 | 468 |
| 175 | 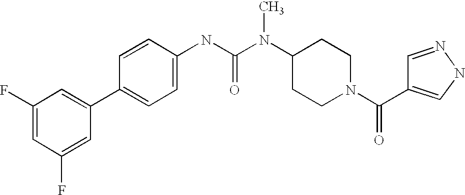 | 440 |
| 176 | 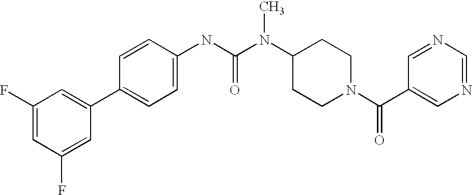 | 452 |
| 177 | 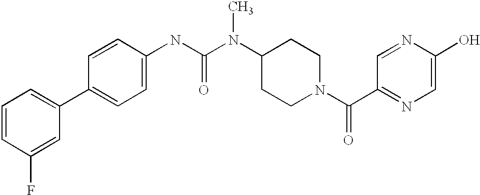 | 450 |
| 178 | 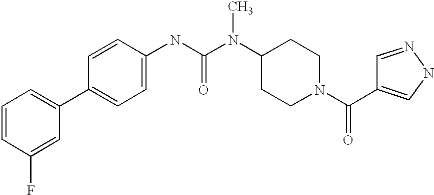 | 422 |
| 179 | 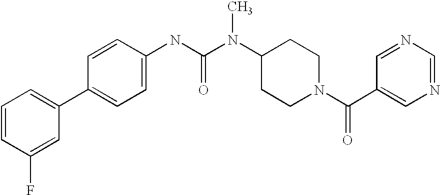 | 434 |
| 180 | 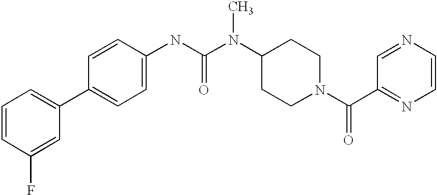 | 434 |
| 181 | 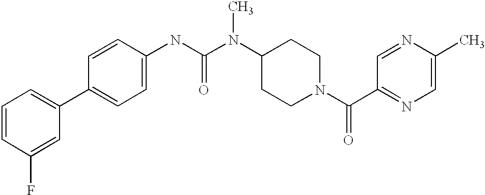 | 448 |
| 182 | 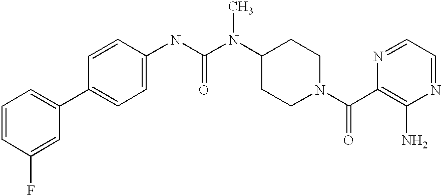 | 449 |
| 183 | 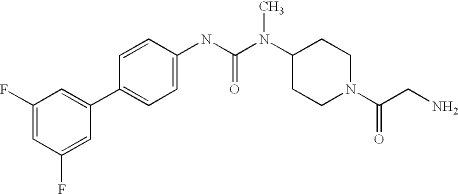 | 403 |
| 184 | 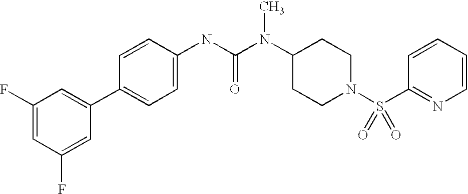 | 487 |
| 185 | 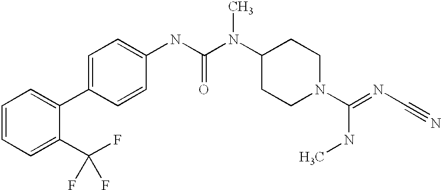 | 459 |
| 186 | 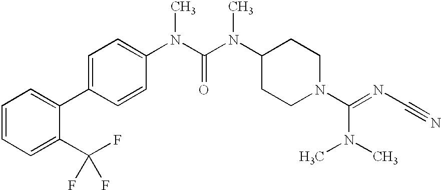 | 487 |
| 187 | 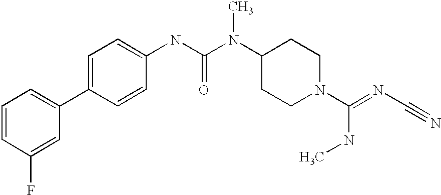 | 409 |
| 188 | 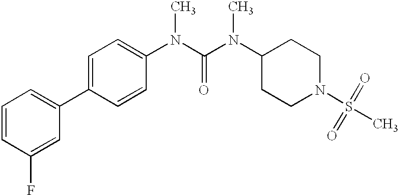 | 420 |
| 189 | 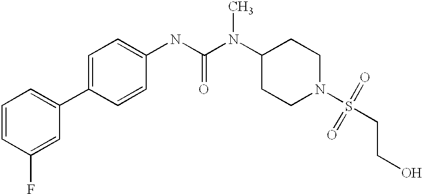 | 436 |
| 190 | 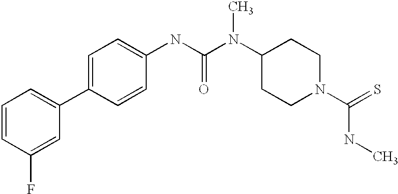 | 401 |
| 191 | 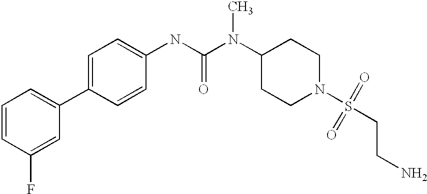 | 435 |
| 192 | 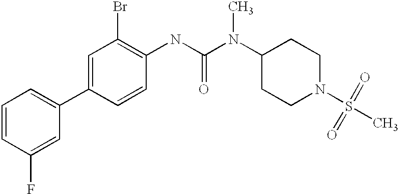 | 485 |
| 193 | 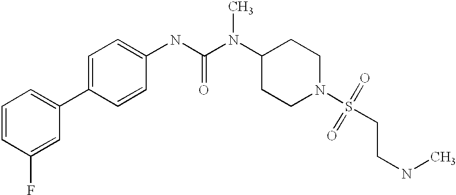 | 449 |
| 194 | 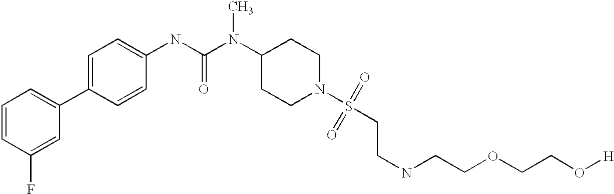 | 523 |
| 195 | 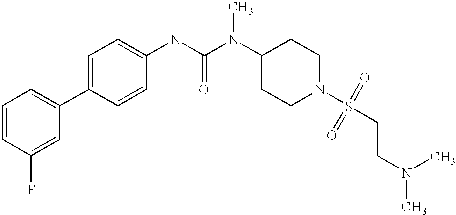 | 463 |
| 196 | 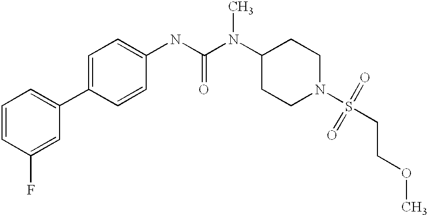 | 450 |
| 197 | 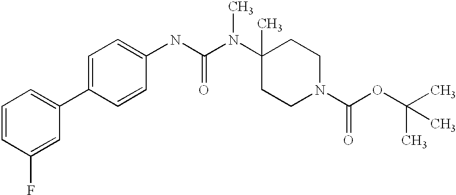 | 442 |
| 198 | 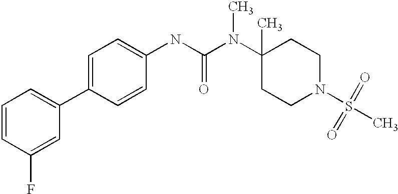 | 420 |
| 199 | 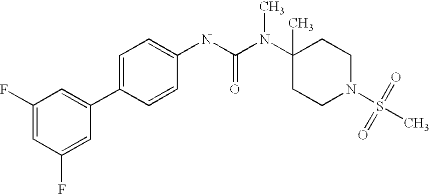 | 438 |
| 200 | 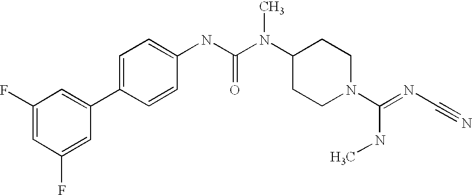 | 427 |
| 201 | 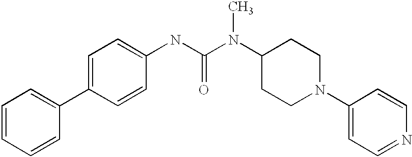 | 387.1 |
| 202 | 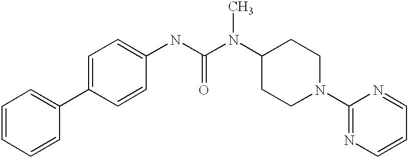 | 388.1 |
| 203 | 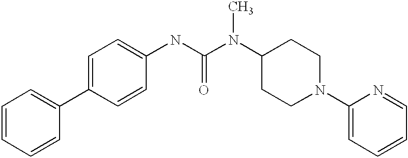 | 387.1 |
| 204 | 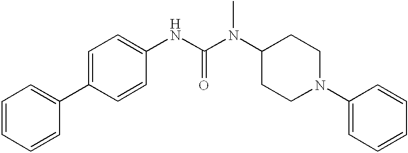 | 386.1 |
| 205 | 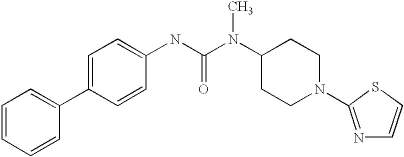 | 393.1 |
| 206 | 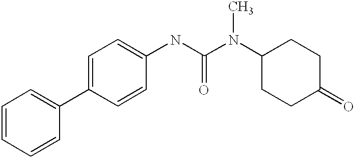 | 323.1 |
| 207 | 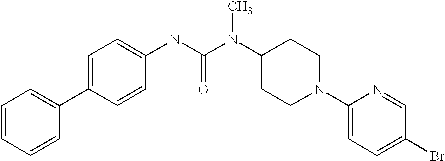 | 465.1, 467.1 |
| 208 | 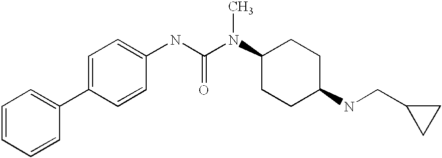 | 378.1 |
| 209 | 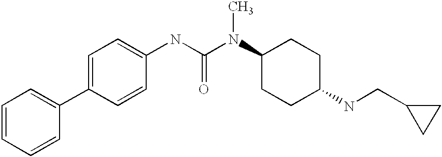 | 378.1 |
| 210 | 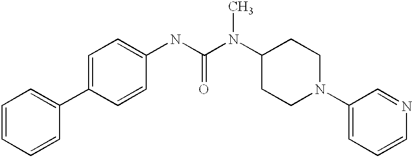 | 387.1 |
| 211 | 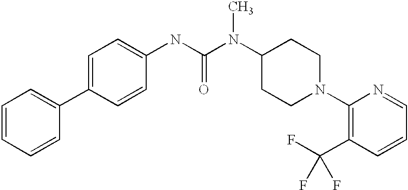 | 455.1 |
| 212 | 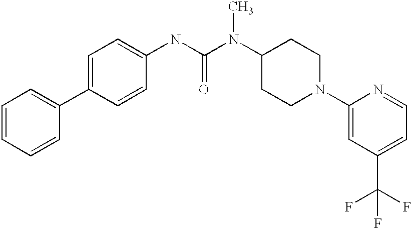 | 455.1 |
| 213 | 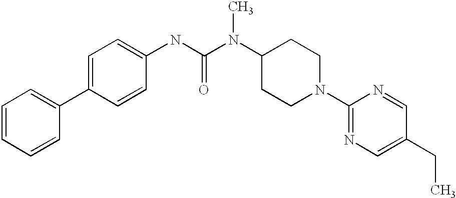 | 416.1 |
| 214 | 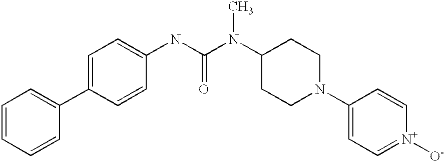 | 403.1 |
| 215 | 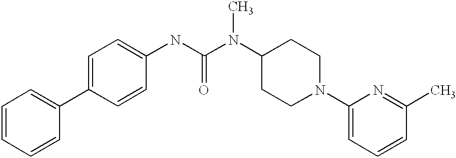 | 401.1 |
| 216 | 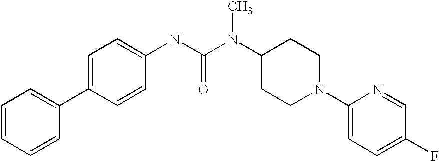 | 405.1 |
| 217 | 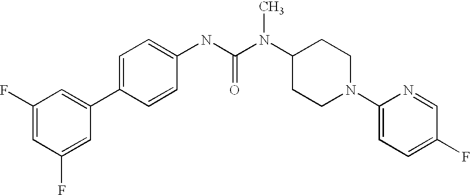 | 441.1 |
| 218 | 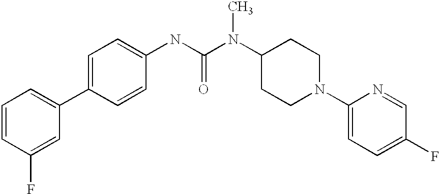 | 423.1 |
| 219 | 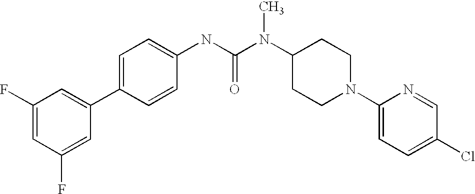 | 457.1 |
| 220 | 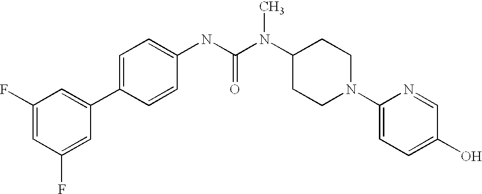 | 439.1 |
| 221 | 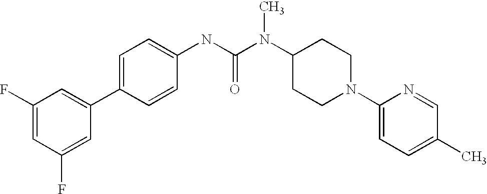 | 437.1 |
| 222 | 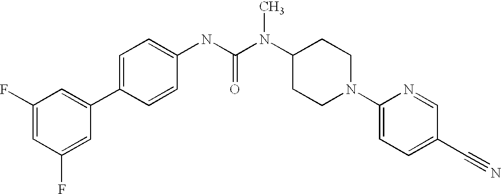 | 448.1 |
| 223 | 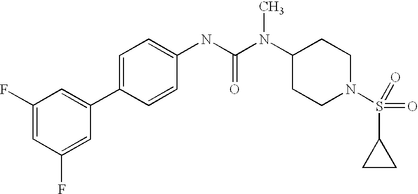 | 450.1 |
| 224 | 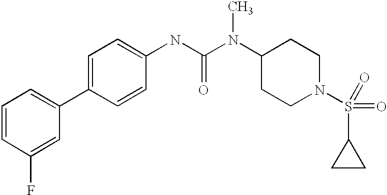 | 432.1 |
| 225 | 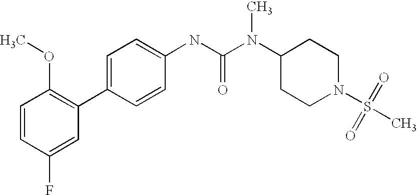 | 436.1 |
| 226 | 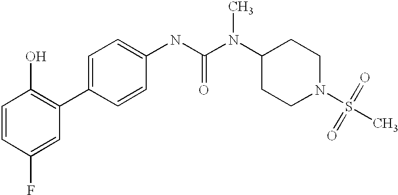 | 422.1 |
| 227 | 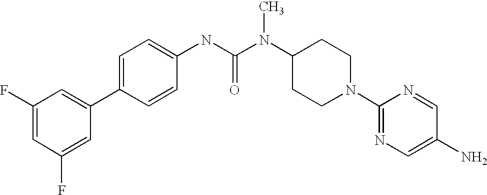 | 439.1 |
| 228 | 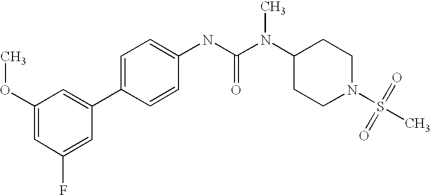 | 436.1 |
| 229 | 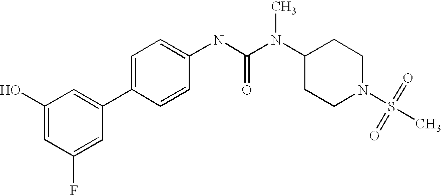 | 422.1 |
| 230 | 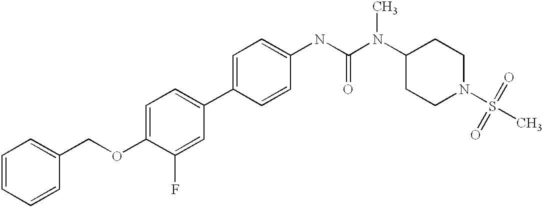 | 512.1 |
| 231 | 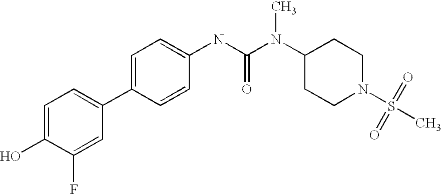 | 422.1 |
| 232 | 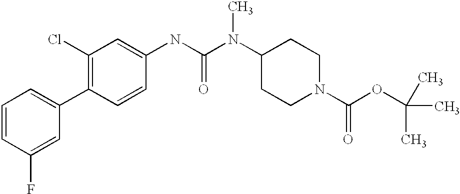 | 462.1 |
| 233 | 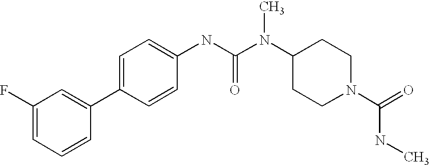 | 385 |
| 234 | 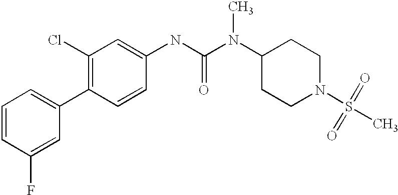 | 440.1 |
| 235 | 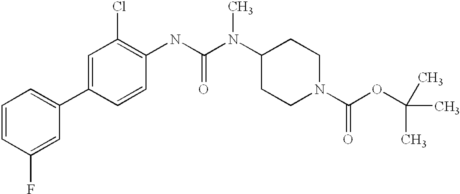 | 462.1 |
| 236 | 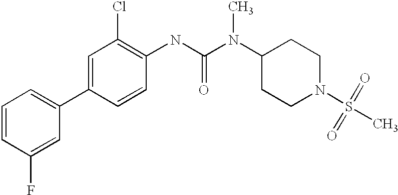 | 440.1 |
| 237 | 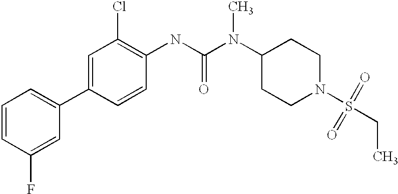 | 454.1 |
| 238 | 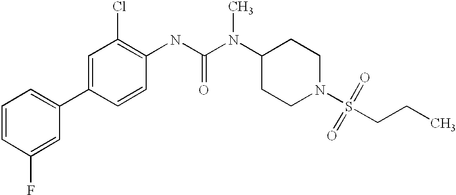 | 468.1 |
| 239 | 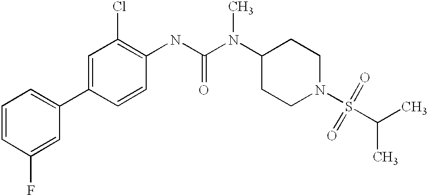 | 468.1 |
| 240 | 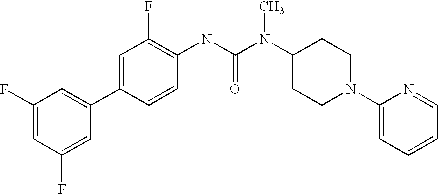 | 441 |
| 241 | 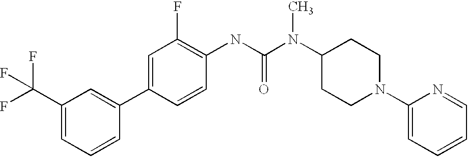 | 473 |
| 242 | 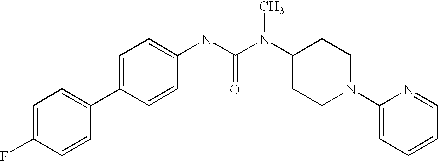 | 405 |
| 243 | 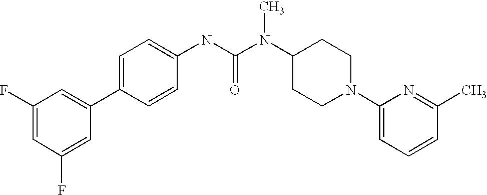 | 437 |
| 244 | 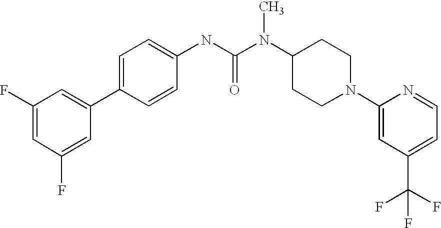 | 491 |
| 245 | 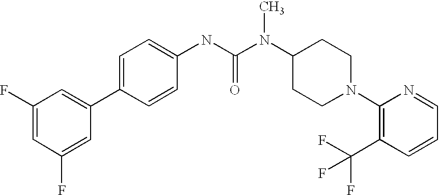 | 491 |
| 246 | 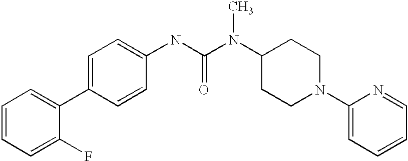 | 405 |
| 247 | 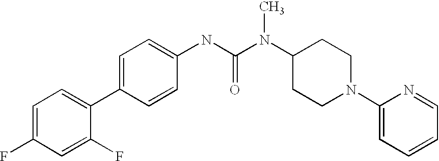 | 423 |
| 248 | 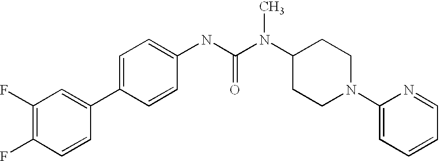 | 423 |
| 249 | 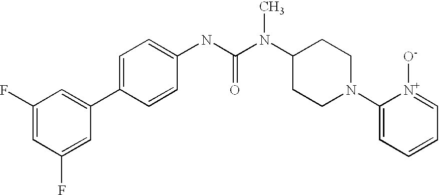 | 439 |
| 250 | 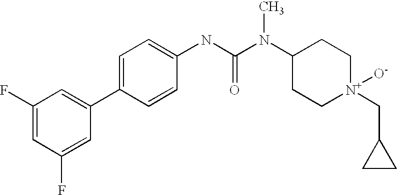 | 416 |
| 251 | 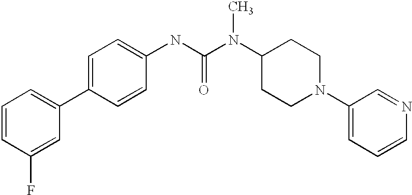 | 405 |
| 252 | 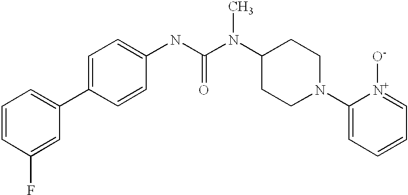 | 421 |
| 253 | 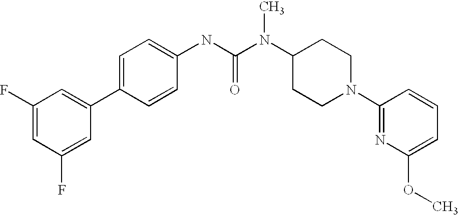 | 453 |
| 254 | 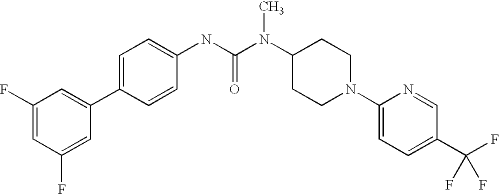 | 491 |
| 255 | 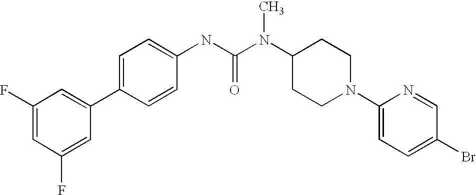 | 501 |
| 256 | 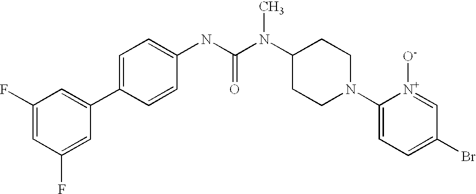 | 517 |
| 257 | 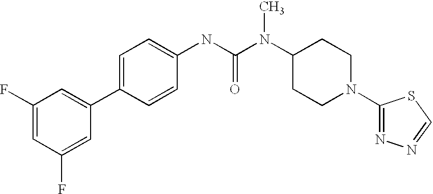 | 430 |
| 258 | 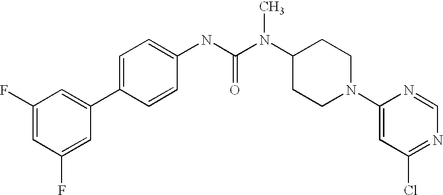 | 458 |
| 259 | 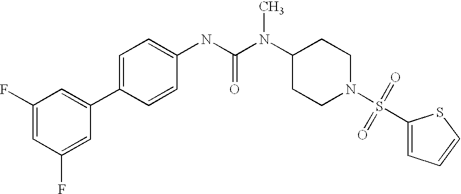 | 492 |
| 260 | 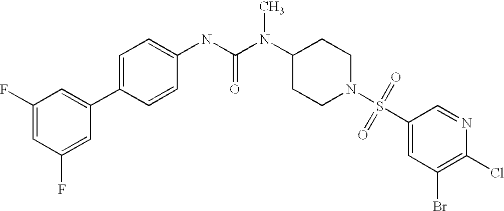 | 599 |
| 261 | 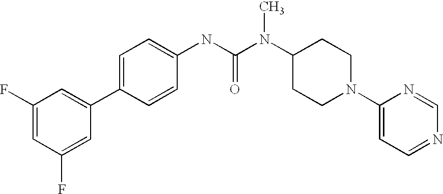 | 424 |
| 262 | 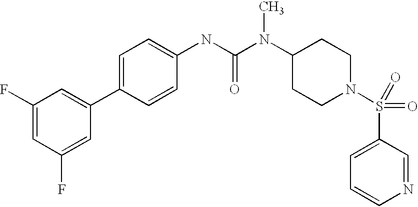 | 487 |
| 263 | 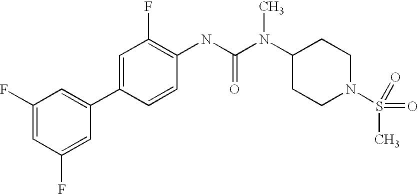 | 442 |
| 264 | 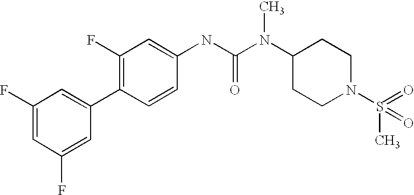 | 442 |
| 265 | 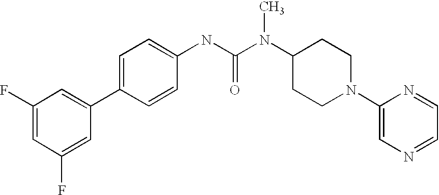 | 424 |
| 266 | 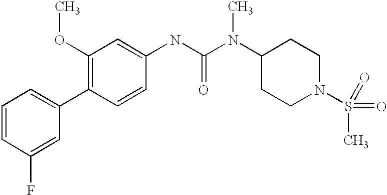 | 436 |
| 267 | 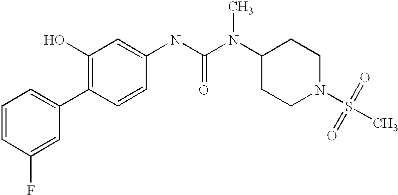 | 422 |
| 268 | 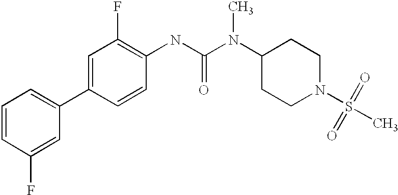 | 424 |
| 269 | 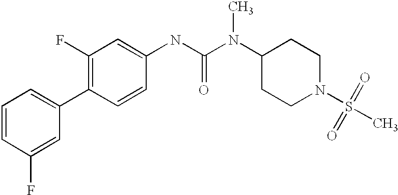 | 424 |
| 270 | 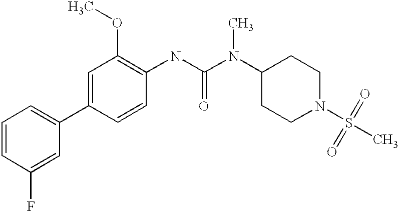 | 436 |
| 271 | 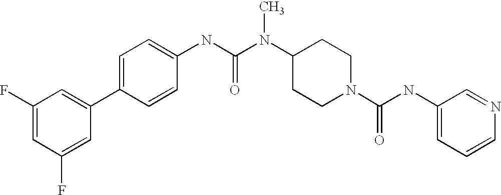 | 466 |
| 272 | 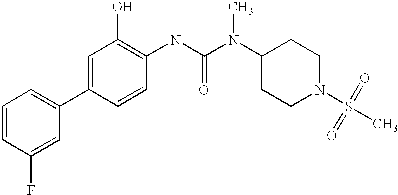 | 422 |
| 273 | 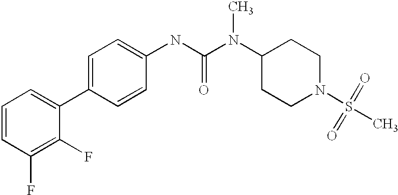 | 424 |
| 274 | 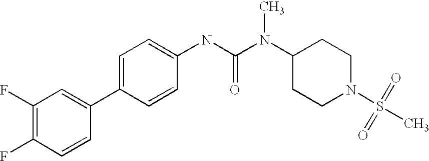 | 424 |
| 275 | 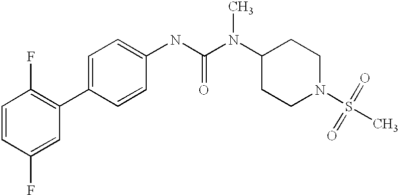 | 424 |
| 276 | 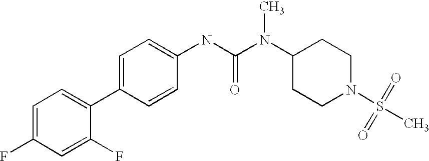 | 424 |
| 277 | 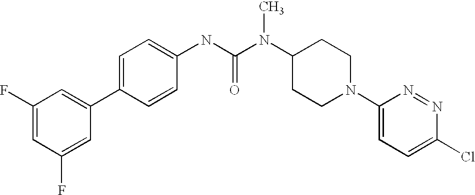 | 458 |
| 278 | 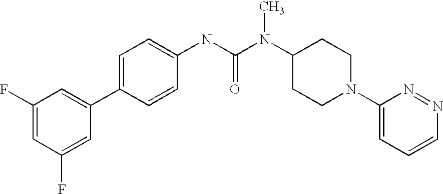 | 424 |
| 279 | 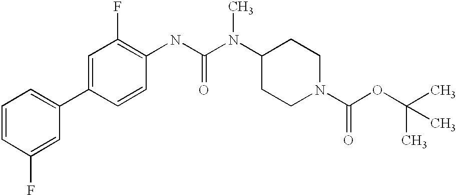 | 446 |
| 280 | 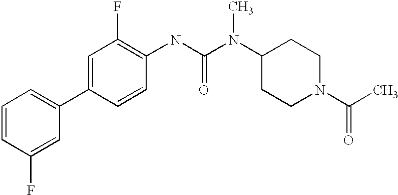 | 388 |
| 281 | 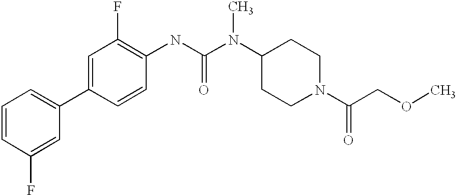 | 418 |
| 282 | 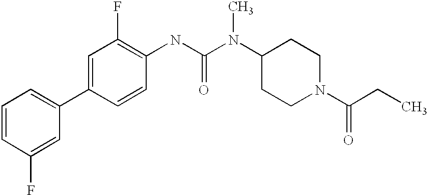 | 402 |
| 283 | 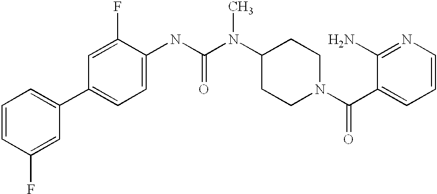 | 466 |
| 284 | 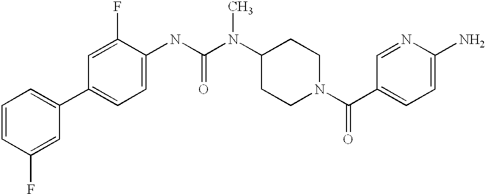 | 466 |
| 285 | 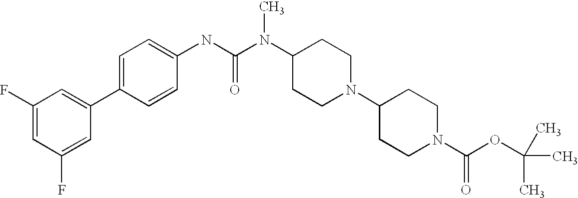 | 529 |
| 286 | 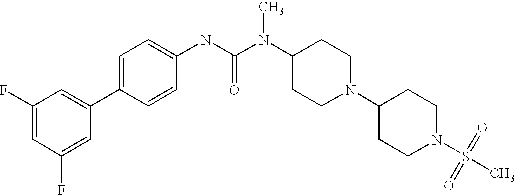 | 507 |
| 287 | 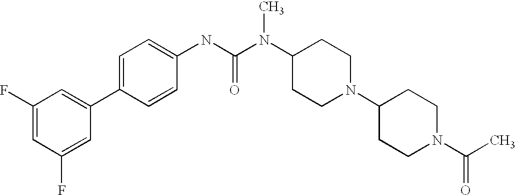 | 471 |
| 288 | 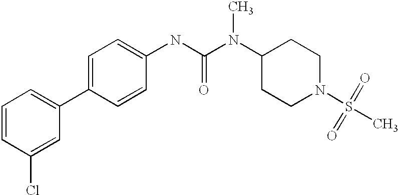 | 422 |
| 289 | 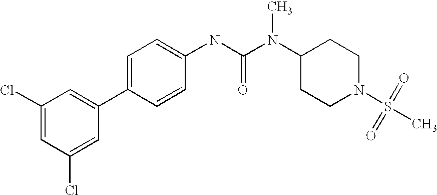 | 456 |
| 290 | 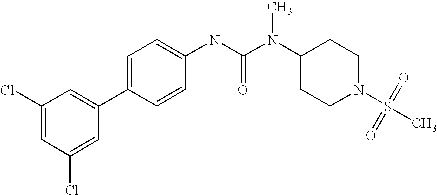 | 456 |
| 291 | 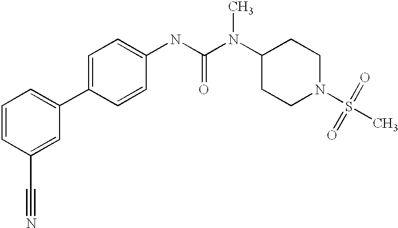 | 413 |
| 292 | 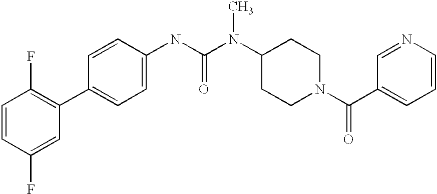 | 451 |
| 293 | 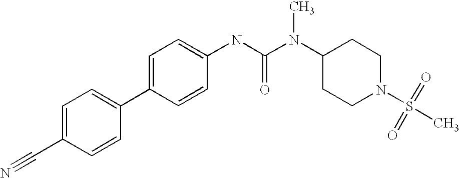 | 413 |
| 294 | 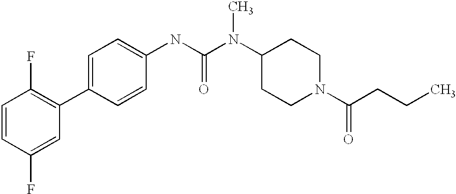 | 416 |
| 295 | 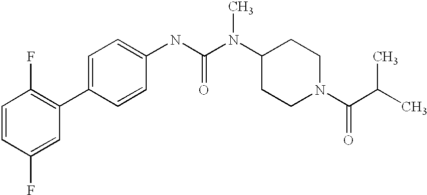 | 416 |
| 296 | 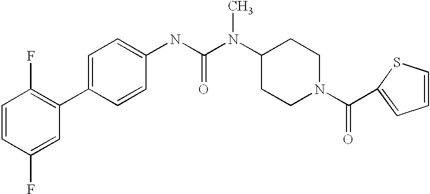 | 456 |
| 297 | 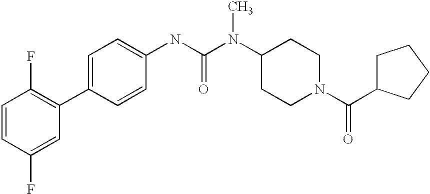 | 442 |
| 298 | 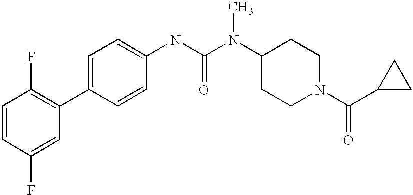 | 414 |
| 299 | 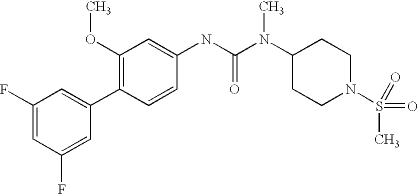 | 454 |
| 300 | 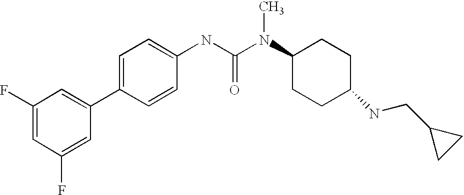 | 414 |
| 301 | 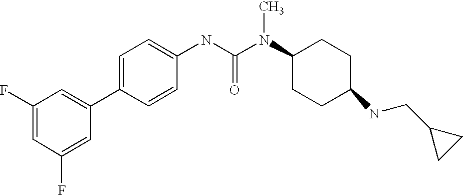 | 414 |
| 302 | 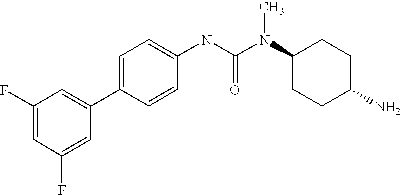 | 360 |
| 303 | 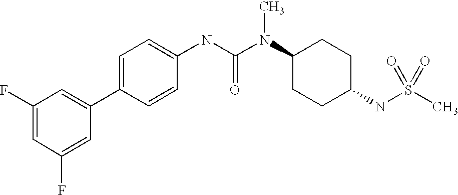 | 438 |
| 304 | 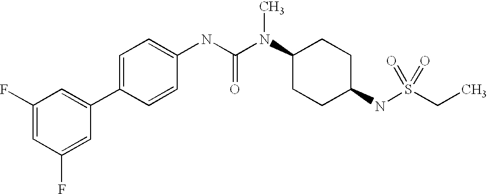 | 452 |
| 305 | 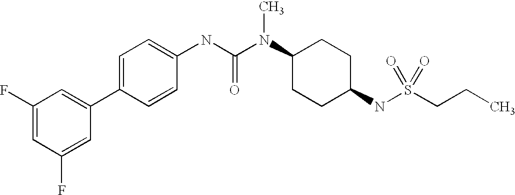 | 466 |
| 306 | 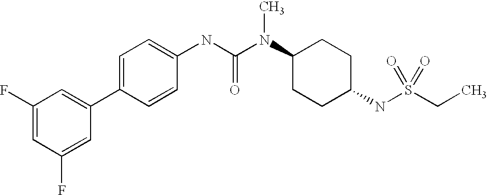 | 452 |
| 307 | 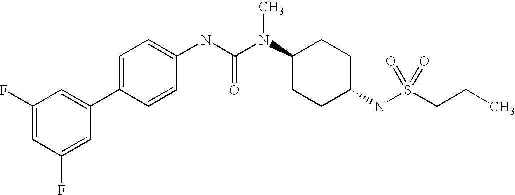 | 466 |
| 308 | 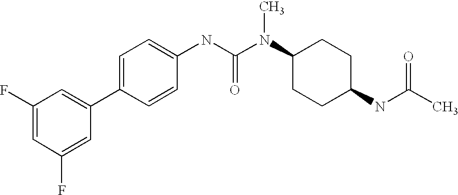 | 402 |
| 309 | 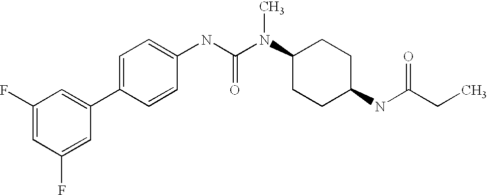 | 416 |
| 310 | 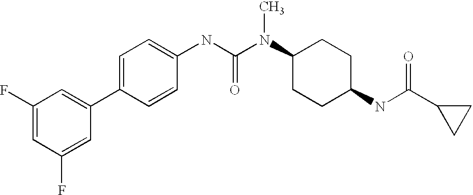 | 428 |
| 311 | 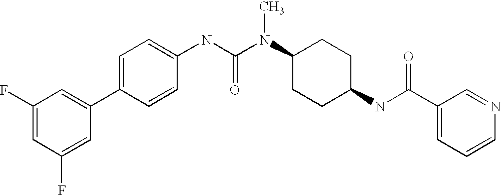 | 465 |
| 312 | 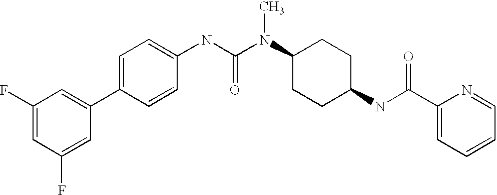 | 465 |
| 314 | 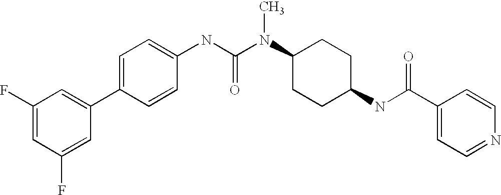 | 465 |
| 315 | 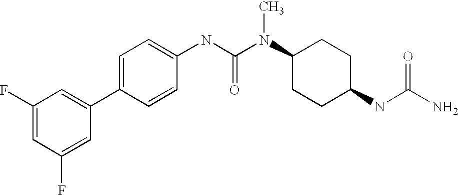 | 403 |
| 316 | 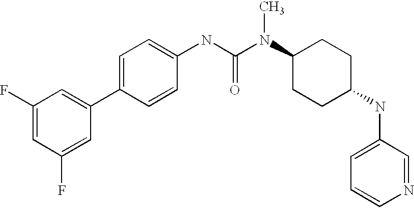 | 437 |
| 317 | 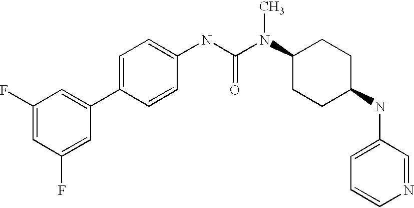 | 437 |
| 318 | 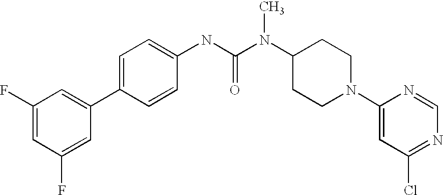 | 458 |
| 319 | 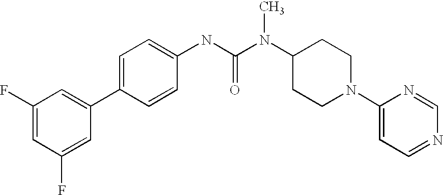 | 424 |
| 320 | 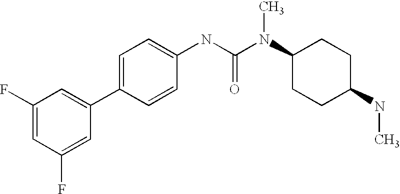 | 374 |
| 321 | 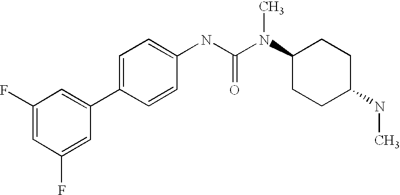 | 374 |
| 322 | 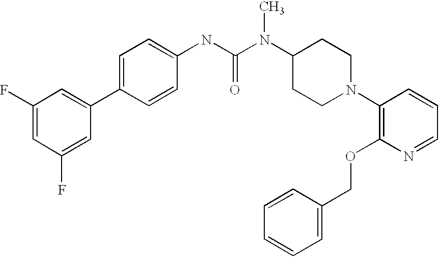 | 529 |
| 323 | 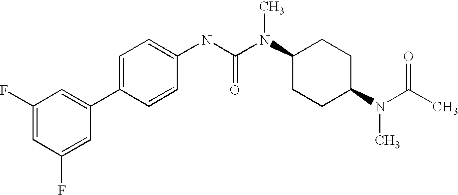 | 416 |
| 324 | 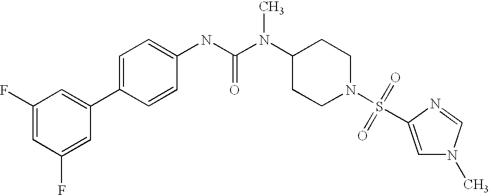 | 490 |
| 325 | 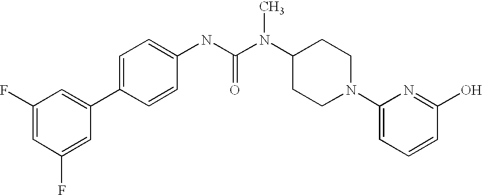 | 439 |
| 326 | 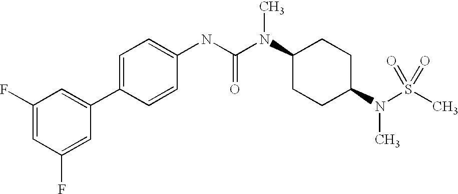 | 452 |
| 327 | 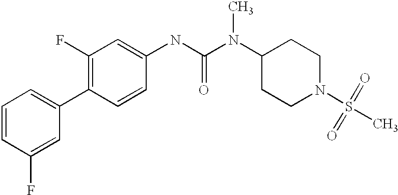 | 424 |
| 328 | 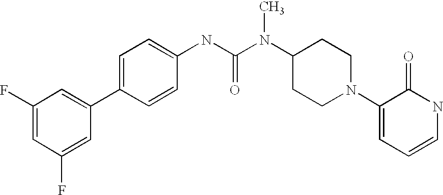 | 439 |
| 329 | 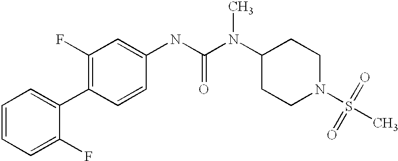 | 424 |
| 330 | 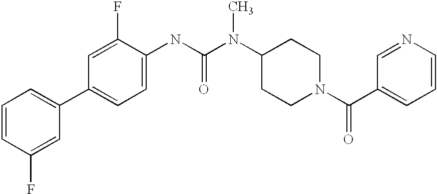 | 451 |
| 331 | 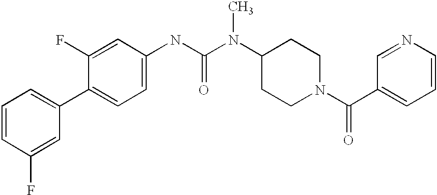 | 451 |
| 332 | 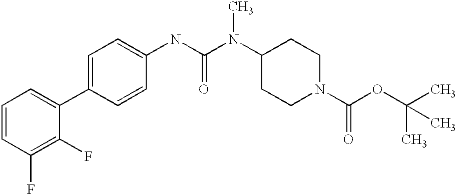 | 446 |
| 333 | 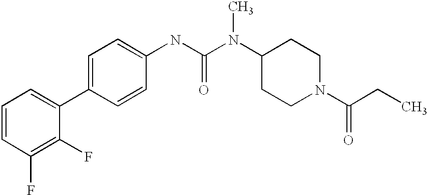 | 402 |
| 334 | 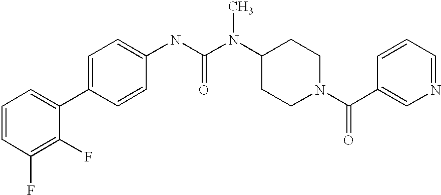 | 451 |
| 335 | 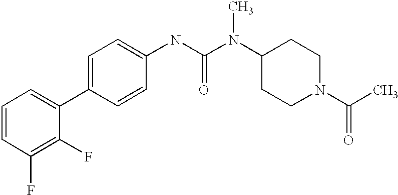 | 388 |
| 336 | 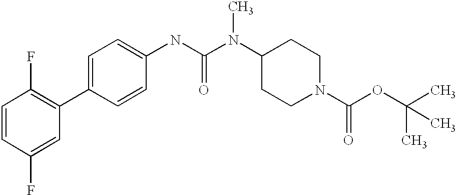 | 446 |
| 337 | 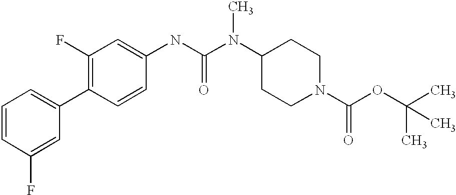 | 446 |
| 338 | 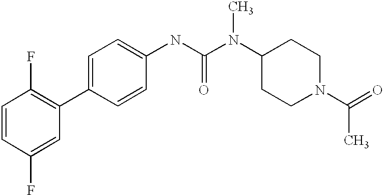 | 388 |
| 339 | 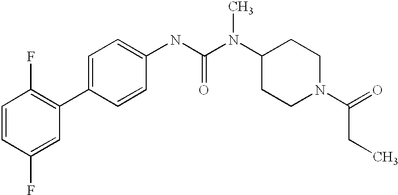 | 402 |
| 340 | 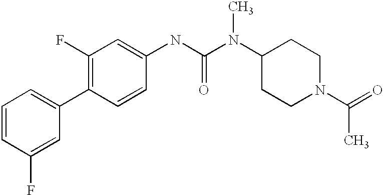 | 388 |
| 341 | 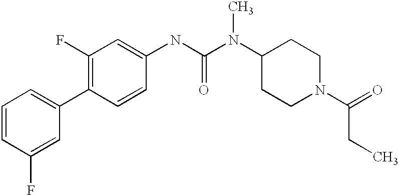 | 402 |
| 342 | 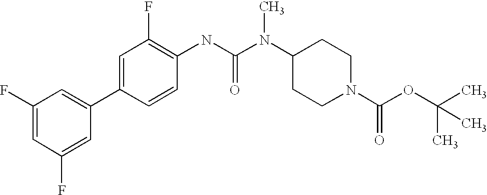 | 464 |

What is claimed:

1. A compound having the structural formula I: 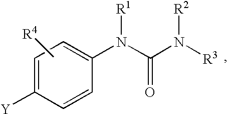
including its N-oxides, wherein 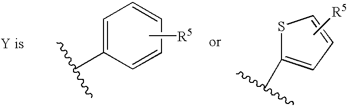
R¹ is H or (C₁-C₆)alkyl;
R² is H, (C₁-C₆)alkyl, (C₃-C₉)cycloalkyl or (C₃-C₇)cycloalkyl(C₁-C₆)alkyl; 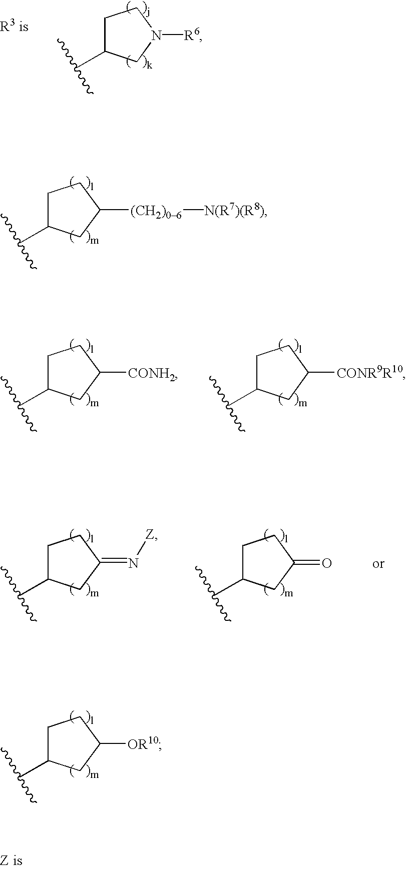
OR¹⁰, —N(R⁹)(R¹⁰) or —NH₂;
j is 0, 1 or 2;
k is 1 or 2;
l is 0, 1 or 2;
m is 0, 1 or 2;
R⁴ is 1-3 substituents independently selected from the group consisting of H, —OH, halogen, haloalkyl, (C₁-C₆)alkyl, (C₃-C₇)cycloalkyl, (C₃-C₇)cycloalkyl(C₁-C₆)alkyl, —CN, —O(C₁-C₆)alkyl, —O(C₃-C₇)cycloalkyl, —O(C₁-C₆)alkyl(C₃-C₇)cycloalkyl, —S(C₁-C₆)alkyl, —S(C₃-C₇)cycloalkyl, —S(C₁-C₆)alkyl(C₃-C₇)cycloalkyl, —NH₂, —NR⁹R¹⁰, —NO₂, —CONH₂, —CONR⁹R¹⁰ and NR²COR¹⁰;
R⁵ is 1-3 substituents independently selected from the group consisting of H, halogen, —OH, haloalkyl, haloalkoxy, —CN, —NO₂, (C₁-C₆)alkyl, (C₃-C₇)cycloalkyl, (C₃-C₇)cycloalkyl(C₁-C₆)alkyl, —O(C₁-C₆)alkyl, —O(C₃-C₇)cycloalkyl, —O(C₁-C₆)alkyl(C₃-C₇)cycloalkyl, —CONH₂ and —CONR⁹R¹⁰;
R⁶ is —SO₂(C₁-C₆)alkyl, —SO₂(C₃-C₇)cycloalkyl, —SO₂(C₁-C₆)alkyl(C₃-C₇)cycloalkyl, —SO₂(C₁-C₆)haloalkyl, —SO₂(hydroxy(C₂-C₆)alkyl), —SO₂(amino(C₂-C₆)alkyl), —SO₂(alkoxy(C₂-C₆)alkyl), —SO₂(alkylamino(C₂-C₆)alkyl), —SO₂(dialkylamino(C₂-C₆)alkyl), —SO₂(aryl), —SO₂(heteroaryl), —SO₂(aryl(C₂-C₆-alkyl), —SO₂NH₂, —SO₂NR⁹R¹⁰, —C(O)(C₁-C₆)alkyl, —C(O)(C₃-C₇)cycloalkyl, —C(O)(C₃-C₇)cycloalkyl(C₁-C₆)alkyl, —C(O)aryl, —C(O)heteroaryl, —C(O)NR⁹R¹⁰, —C(O)NH₂, —C(S)NR⁹R¹⁰, —C(S)NH₂, aryl, heteroaryl, —(CH₂)ₙC(O)NH₂, —(CH₂)ₙC(O)NR⁹R¹⁰, —C(═NCN)alkylthio, —C(═NCN)NR⁹R¹⁰, (C₁-C₆)alkyl, (C₃-C₇)cycloalkyl, (C₃-C₇)cycloalkyl(C₁-C₆)alkyl, aryl(C₁-C₆)alkyl, heteroaryl(C₁-C₆)alkyl or —C(O)OR⁹, n=1 to 6;
R⁷=H or alkyl;
R⁸ is H, (C₁-C₆)alkyl, (C₃-C₇)cycloalkyl, (C₃-C₇)cycloalkyl(C₁-C₆)alkyl, aryl heteroaryl, —SO₂(C₁-C₆)alkyl, —SO₂(C₃-C₇)cycloalkyl, —SO₂(C₁-C₆)alkyl(C₃-C₇)cycloalkyl, —SO₂(C₁-C₆)haloalkyl or —SO₂(aryl);
R⁹ is (C₁-C₆)alkyl, (C₃-C₇)cycloalkyl, (C₃-C₇)cycloalkyl(C₁-C₆)alkyl, aryl(C₁-C₆)alkyl, aryl or heteroaryl; and,
R¹⁰ is hydrogen, (C₁-C₆)alkyl, (C₃-C₇)cycloalkyl, (C₃-C₇)cycloalkyl(C₁-C₆)alkyl, aryl(C₁-C₆)alkyl, aryl or heteroaryl;
or R⁹ and R¹⁰ taken together can form a 4-7 membered ring containing 1 or 2 heteroatoms;
or its pharmaceutically acceptable addition salt and/or hydrate thereof, or prodrug thereof, or where applicable, a geometric or optical isomer or a racemic mixture thereof.

2. A compound of claim 1 wherein

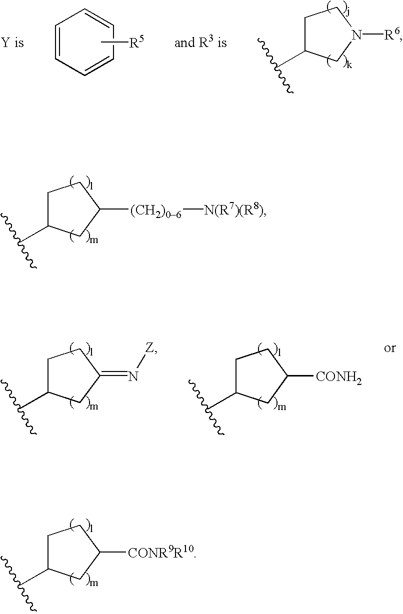

3. A compound of claim 2 wherein R⁵ is 1-3 substituents independently selected from the group consisting of H, halogen, haloalkyl, alkoxy and haloalkoxy and the sum of j and k is 1, 2 or 3.

4. A compound of claim 2 wherein R⁶ is SO₂(C₁-C₆)alkyl, SO₂hydroxy(C₂-C₆)alkyl, SO₂(C₃-C₇)cycloalkyl, SO₂NR⁹R¹⁰ or SO₂NH₂.

5. A compound of claim 1 selected from the group consisting of 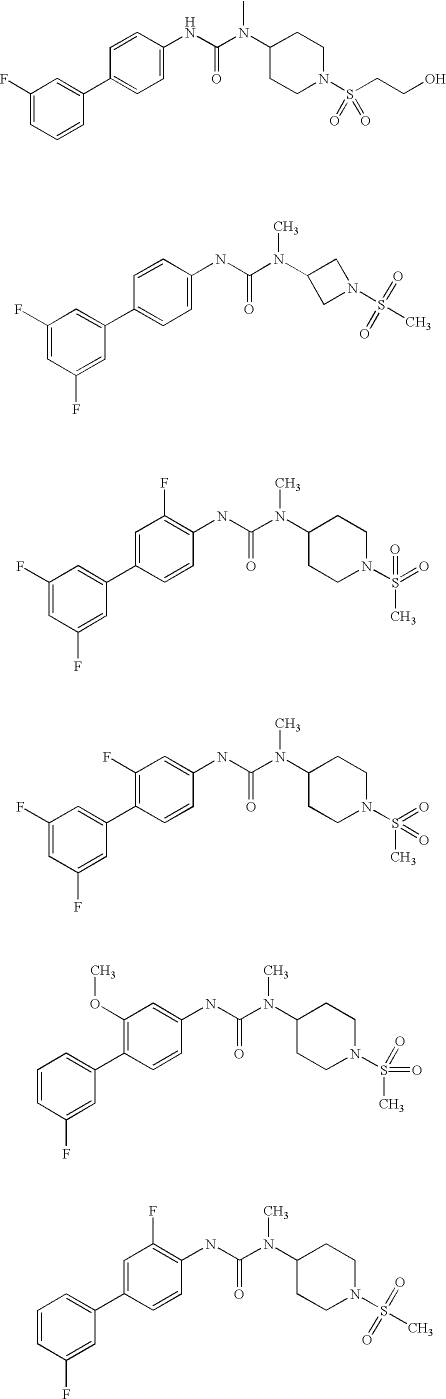
pharmaceutically acceptable addition salts and/or hydrates thereof, or prodrugs thereof, or where applicable, geometric or optical isomers or a racemic mixtures thereof.

6. A compound of claim 1, wherein the compound is 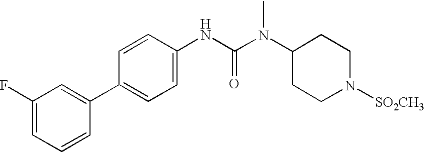
or its pharmaceutically acceptable addition salt and/or hydrate thereof, or prodrug thereof, or where applicable, a geometric or optical isomer or a racemic mixture thereof.

7. A compound of claim 2 wherein R⁶ is C(O)heteroraryl, C(O)(C₁-C₆)alkyl or C(O)(C₃-C₇)cycloalkyl.

8. A compound of claim 1 selected from the group consisting of 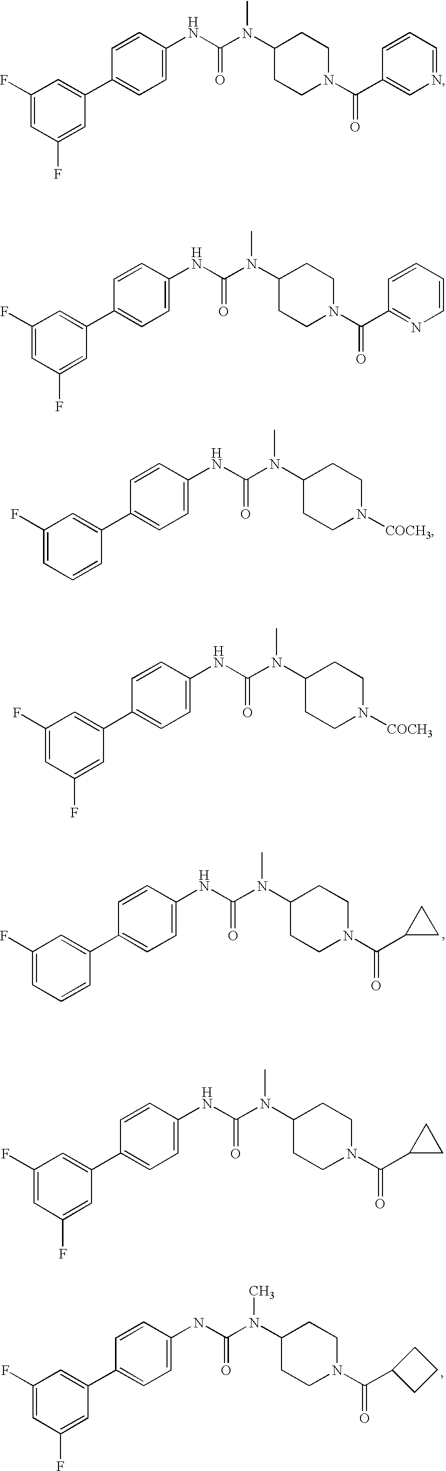
and their pharmaceutically acceptable addition salts and/or hydrates thereof, or prodrugs thereof, or where applicable, geometric or optical isomers or a racemic mixtures thereof.

9. A compound of claim 2 wherein R⁶ is heteroaryl.

10. A compound of claim 1 selected from the group consisting of 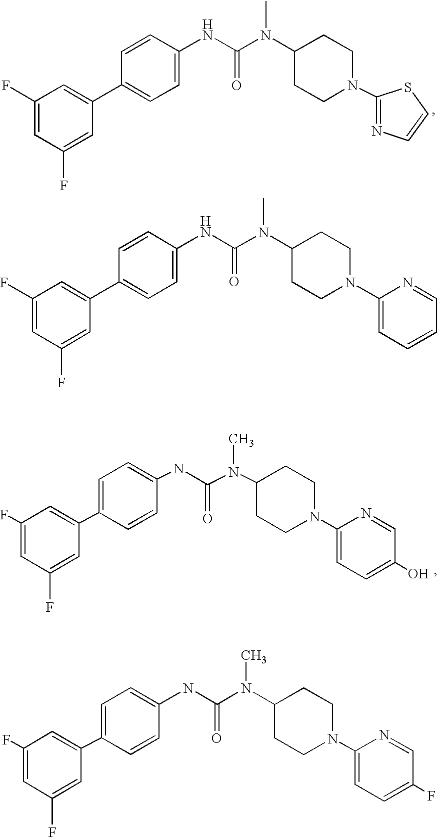
and their pharmaceutically acceptable addition salts and/or hydrates thereof, or prodrugs thereof, or where applicable, geometric or optical isomers or a racemic mixtures thereof.

11. A compound of claim 1 wherein

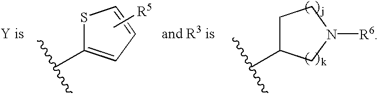

12. A compound of claim 11 wherein R⁵ is 1 to 3 substituents independently selected from the group consisting of H, halogen, haloalkyl and haloalkoxy and the sum of j and k is 1, 2 or 3.

13. A compound of claim 11 wherein R⁶ is SO₂(C₁-C₆)alkyl, SO₂(C₃-C₇)cycloalkyl, SO₂NR⁹R¹⁰ or SO₂NH₂.

14. A compound of the formula 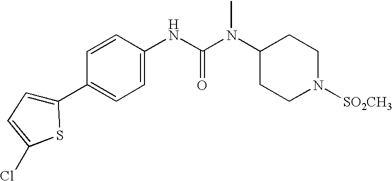
or its pharmaceutically acceptable addition salt and/or hydrate thereof, or prodrug thereof, or where applicable, a geometric or optical isomer or a racemic mixture thereof.

15. A compound of claim 11 wherein R⁶ is C(O)heteroaryl, C(O)(C₁-C₆)alkyl or C(O)(C₃-C₇)cycloalkyl.

16. A compound of claim 1 selected from the group consisting of 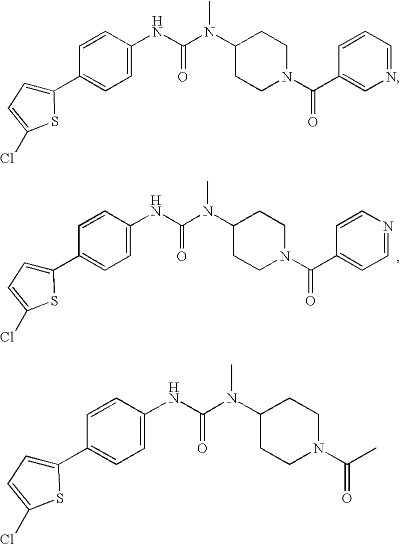
and their pharmaceutically acceptable addition salts and/or hydrates thereof, or prodrugs thereof, or where applicable, geometric or optical isomers or a racemic mixtures thereof.

17. A compound of claim 11 wherein R⁶ is heteroaryl.

18. A compound of claim 1 selected from the group consisting of those having the structural formulas set forth in the following table, and the pharmaceutically acceptable addition salts and/or hydrates thereof, or prodrugs thereof, or where applicable, geometric or optical isomers or a racemic mixtures thereof:

| Y | R¹ | R² | R³ | R⁴ |
|---|---|---|---|---|
| 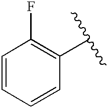 | —H | —CH₃ | 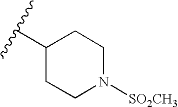 | —H |
| 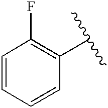 | —H | —CH₃ | 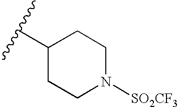 | —H |
| 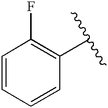 | —H | —CH₃ | 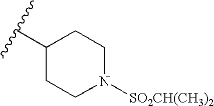 | —H |
| 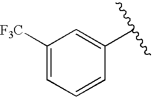 | —H | —CH₃ | 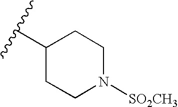 | —H |
| 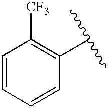 | —H | —CH₃ | 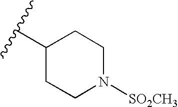 | —H |
| 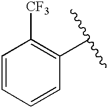 | —H | —CH₃ | 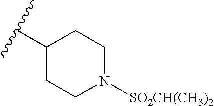 | —H |
| 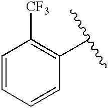 | —H | —CH₃ | 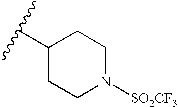 | —H |
| 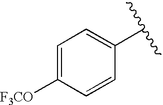 | —H | —CH₃ | 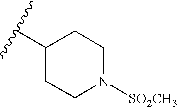 | —H |
| 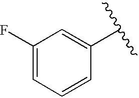 | —H | —CH₃ | 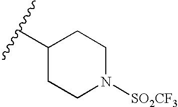 | —H |
| 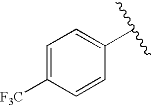 | —H | —CH₃ | 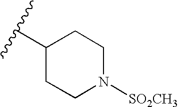 | —H |
| 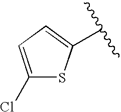 | —H | —CH₃ | 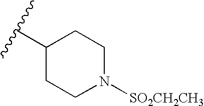 | —H |
| 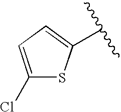 | —H | —CH₃ | 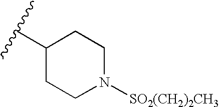 | —H |
| 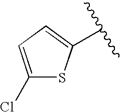 | —H | —CH₃ | 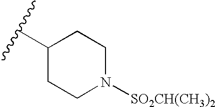 | —H |
| 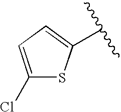 | —H | —CH₃ | 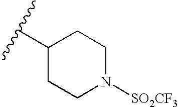 | —H |
| 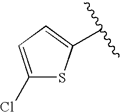 | —H | —CH₃ | 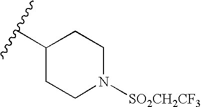 | —H |
| 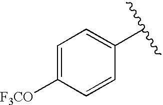 | —H | —CH₃ | 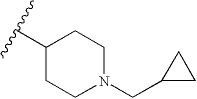 | —H |
| 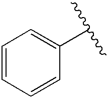 | —H | —CH₃ | 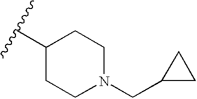 | —H |
| 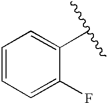 | —H | —CH₃ | 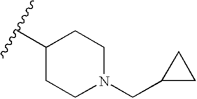 | —H |
| 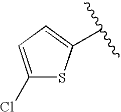 | —H | —CH₃ | 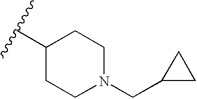 | —H |
| 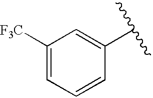 | —H | —CH₃ | 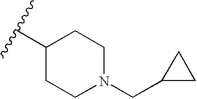 | —H |
| 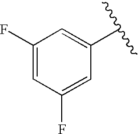 | —H | —CH₃ | 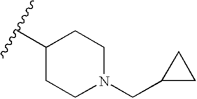 | —H |
| 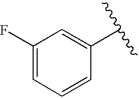 | —H | —CH₃ | 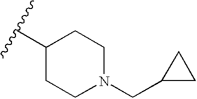 | —H |
| 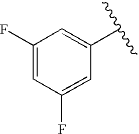 | —H | —CH₃ | 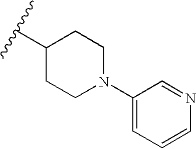 | —H |
| 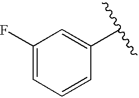 | —H | —CH₃ | 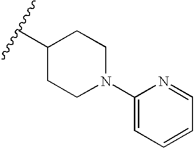 | —H |
| 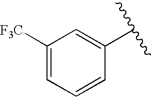 | —H | —CH₃ | 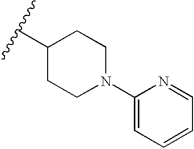 | —H |
| 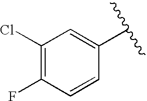 | —H | —CH₃ | 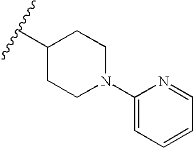 | —H |
| 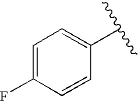 | —H | —CH₃ | 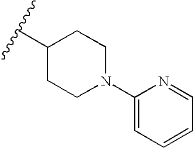 | —H |
| 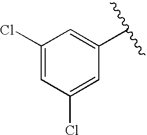 | —H | —CH₃ | 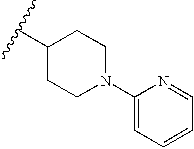 | —H |
| 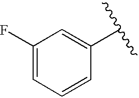 | —H | —CH₃ | 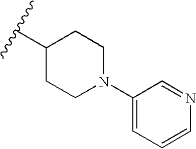 | —H |
| 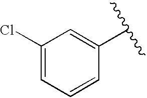 | —H | —CH₃ | 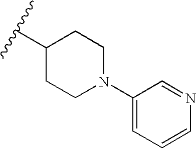 | —H |
| 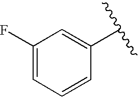 | —H | —CH₃ | 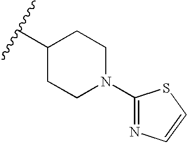 | —H |
| 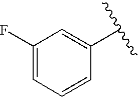 | —H | —CH₃ | 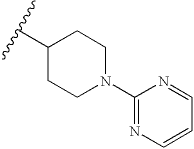 | —H |
| 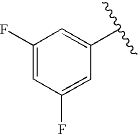 | —H | —CH₃ | 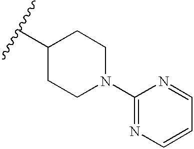 | —H |
| 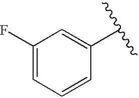 | —H | —CH₃ | 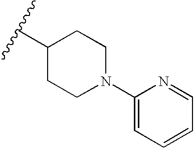 | 2-F |
| 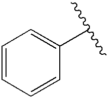 | —H | —CH₃ | 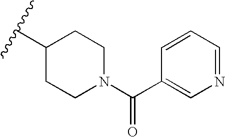 | —H |
| 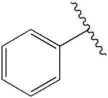 | —H | —CH₃ | 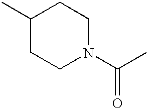 | —H |
| 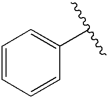 | —H | —CH₃ | 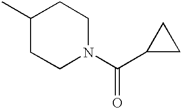 | —H |
| 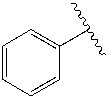 | —H | —CH₃ | 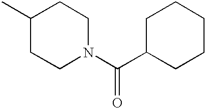 | —H |
| 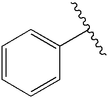 | —H | —CH₃ | 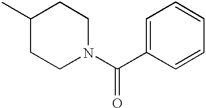 | —H |
| 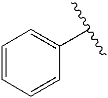 | —H | —CH₃ | 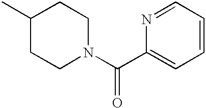 | —H |
| 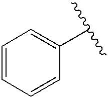 | —H | —CH₃ | 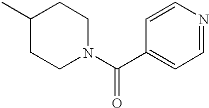 | —H |
| 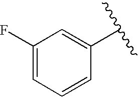 | —H | —CH₃ | 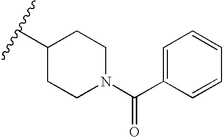 | —H |
| 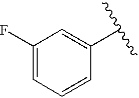 | —H | —CH₃ | 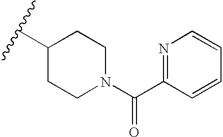 | —H |
| 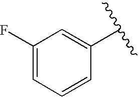 | —H | —CH₃ | 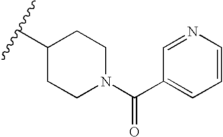 | —H |
| 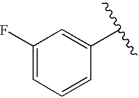 | —H | —CH₃ | 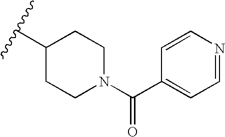 | —H |
| 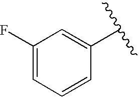 | —H | —CH₃ | 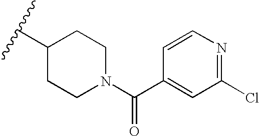 | —H |
| 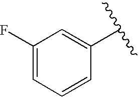 | —H | —CH₃ | 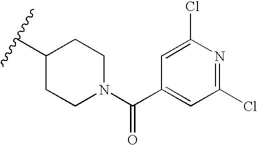 | —H |
| 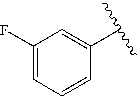 | —H | —CH₃ | 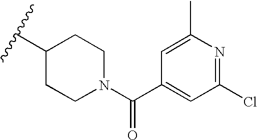 | —H |
| 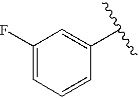 | —H | —CH₃ | 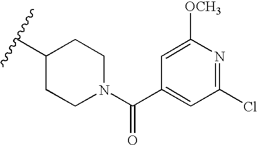 | —H |
| 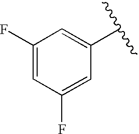 | —H | —CH₃ | 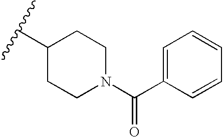 | —H |
| 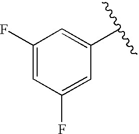 | —H | —CH₃ | 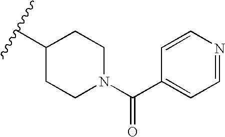 | —H |
| 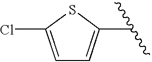 | —H | —CH₃ | 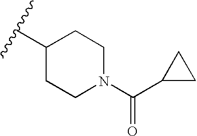 | —H |
| 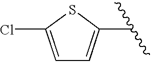 | —H | —CH₃ | 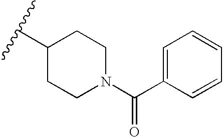 | —H |
| 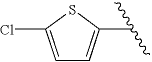 | —H | —CH₃ | 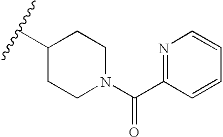 | —H |
| 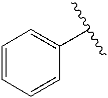 | —H | —CH₃ | 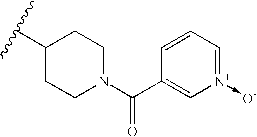 | —H |
| 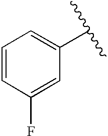 | —H | —CH₃ | —CH₂CONH₂ | —H |
| 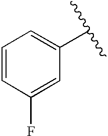 | —H | —CH₃ | 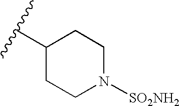 | —H |
| 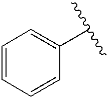 | —H | —CH₃ | 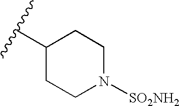 | —H |
| 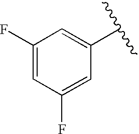 | —H | —CH₃ | 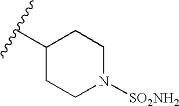 | —H |
| 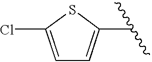 | —H | —CH₃ | 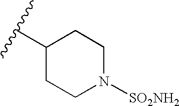 | —H |
| 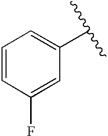 | —H | —CH₃ | 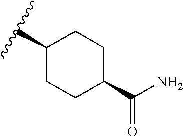 | —H |
| 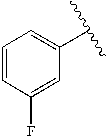 | —H | —CH₃ | 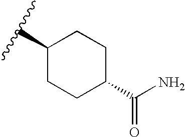 | —H |
| 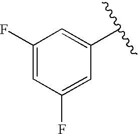 | —H | —CH₃ | 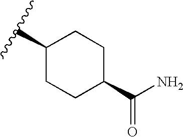 | —H |
| 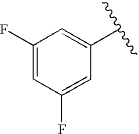 | —H | —CH₃ | 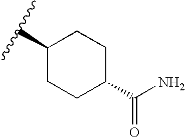 | —H |
| 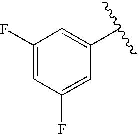 | —H | —CH₃ | 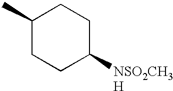 | —H |
| 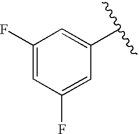 | —H | —CH₃ | 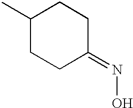 | —H |
| 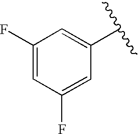 | —H | —CH₃ | 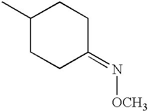 | —H |

19. The compound of claim 1 selected from the compounds of Examples: 29-59, 61-90, 95-216, 218-219, 221-262, 265, 267, 269-294, 296-297, 299-326, 328-337, 340-342 and their pharmaceutically acceptable addition salts and/or hydrates thereof, or prodrugs thereof, or where applicable, geometric or optical isomers or a racemic mixtures thereof.

20. A pharmaceutical composition comprising a compound of formula I as defined in claim 1 in combination with a pharmaceutically acceptable carrier.

21. A method of treating obesity, an eating disorder or diabetes comprising administering an effective amount of a compound of formula 1 as defined in claim 1 to a mammal in need of such treatment. A pharmaceutical composition, which comprises an effective amount of a compound as, defined in claim 1 and a pharmaceutically acceptable carrier thereof.

22. A method of treating metabolic or eating disorders comprising administering to a mammal in need of such treatment a therapeutically effective amount of a compound of claim 1 or a prodrug thereof or a pharmaceutically acceptable salt of said compound or of said prodrug.

23. The method of claim 22 wherein said metabolic disorder is obesity.

24. The method of claim 22 wherein said eating disorder is hyperphagia.

25. A method of treating disorders associated with obesity comprising administering to a mammal in need of such treatment a therapeutically effective amount of a compound of claim 1 or a prodrug thereof or a pharmaceutically acceptable salt of said compound or of said prodrug.

26. The method of claim 25 wherein said disorders associated with obesity are Type II Diabetes, insulin resistance, hyperlipidemia and hypertension.

27. A pharmaceutical composition which comprises a therapeutically effective amount of a composition comprising:
a first compound, said first compound being a compound of claim 1, a prodrug thereof, or a pharmaceutically acceptable salt of said compound or of said prodrug;
a second compound, said second compound being an anti-obesity and/or anorectic agent such as a β₃ agonist, a thryomimetic agent, an anorectic agent or an NPY antagonist; and
a pharmaceutically acceptable carrier thereof.

28. A method of treating a metabolic or eating disorder which comprises administering to a mammal in need of such treatment
an amount of a first compound, said first compound being a compound of claim 1, a prodrug thereof, or a pharmaceutically acceptable salt of said compound or of said prodrug;
a second compound, said second compound being an antiobesity and/or anorectic agent such as a β₃ agonist, a thryomimetic agent, an anorectic agent or an NPY antagonist;
wherein the amounts of the first and second compounds result in a therapeutic effect.

29. A pharmaceutical composition which comprises a therapeutically effective amount of a composition comprising:
a first compound, said first compound being a compound of claim 1, a prodrug thereof, or a pharmaceutically acceptable salt of said compound or of said prodrug;
a second compound, said second compound being an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a PPAR-gamma ligand such as troglitazone, rosaglitazone, pioglitazone, or GW-1929, a sulfonylurea, glipazide, glyburide, or chlorpropamide; and
a pharmaceutically acceptable carrier therefor.

30. A pharmaceutical composition made by combining the compound of claim 1 and a pharmaceutically acceptable carrier therefor.

31. A process for making a pharmaceutical composition comprising combining a compound of claim 1 and a pharmaceutically acceptable carrier.
